SEQUENCE LISTING (84 sequences)

SEQ ID NO: 1 - protein, 11 aa, synthetic construct
  REGION 1..11: note = CDRH1
GYSITSDYAWN

SEQ ID NO: 2 - protein, 16 aa, synthetic construct
  REGION 1..16: note = CDRH2
YISYSGSFSYHPSLKS

SEQ ID NO: 3 - protein, 10 aa, synthetic construct
  REGION 1..10: note = CDRH3
SFSYGYGFAY

SEQ ID NO: 4 - protein, 11 aa, synthetic construct
  REGION 1..11: note = CDRL1
KASQDVSSAVA

SEQ ID NO: 5 - protein, 7 aa, synthetic construct
  REGION 1..7: note = CDRL2
SASYRYT

SEQ ID NO: 6 - protein, 9 aa, synthetic construct
  REGION 1..9: note = CDRL3
QQHYSTPWT

SEQ ID NO: 7-8 - skipped (2 entries)

SEQ ID NO: 9 - protein, 11 aa, synthetic construct
  REGION 1..11: note = CDRH1
GYSITSDYTWN

SEQ ID NO: 10 - protein, 16 aa, synthetic construct
  REGION 1..16: note = CDRH2
YISYSGSFSYNPSLKS

SEQ ID NO: 11 - protein, 16 aa, synthetic construct
  REGION 1..16: note = CDRH2
YISYSGSYSYNPSLKS

SEQ ID NO: 12 - protein, 16 aa, synthetic construct
  REGION 1..16: note = CDRH2
YISYSGSYSYHPSLKS

SEQ ID NO: 13 - protein, 10 aa, synthetic construct
  REGION 1..10: note = CDRH3
SLSYGYGFAY

SEQ ID NO: 14 - protein, 10 aa, synthetic construct
  REGION 1..10: note = CDRH3
SFSYSYGFAY

SEQ ID NO: 15 - protein, 7 aa, synthetic construct
  REGION 1..7: note = CDRL2
SASYRYI

SEQ ID NO: 16 - protein, 11 aa, synthetic construct
  REGION 1..11: note = CDRH1
  VARIANT 9: note = X is A or T
GYSITSDYXWN

SEQ ID NO: 17 - protein, 16 aa, synthetic construct
  REGION 1..16: note = CDRH2
  VARIANT 8: note = X is F or Y
  VARIANT 11: note = X is H or N
YISYSGSXSYXPSLKS

SEQ ID NO: 18 - protein, 10 aa, synthetic construct
  REGION 1..10: note = CDRH3
  VARIANT 2: note = X is L or F
  VARIANT 5: note = X is G, S or A
SXSYXYGFAY

SEQ ID NO: 19 - protein, 7 aa, synthetic construct
  REGION 1..7: note = CDRL2
  VARIANT 7: note = X is T or I
SASYRYX

SEQ ID NO: 20 - protein, 107 aa, synthetic construct
  REGION 1..107: note = Light chain variable region
DIQMTQSPSSLSASVGDRVTITCKASQDVSSAVAWYQQKPGKAPKLLIYSASYRYTGVPSRFSGSGSGTDFTFTISSLQPEDFATYYCQQHYSTPWTFGGGTKVEIK

SEQ ID NO: 21 - protein, 107 aa, synthetic construct
  REGION 1..107: note = Light chain variable region
DIQMTQSPSSLSASVGDRVTITCKASQDVSSAVAWYQQKPGKAPKLLIYSASYRYIGVPSRFSGSGSGTDFTFTISSLQPEDFATYYCQQHYSTPWTFGGGTKVEIK

SEQ ID NO: 22 - protein, 107 aa, synthetic construct
  REGION 1..107: note = Light chain variable region
DIVMTQSPSSLSASVGDRVTITCKASQDVSSAVAWYQQKPGQSPKLLIYSASYRYTGVPDRFSGSGSGTDFTFTISSLQPEDFATYYCQQHYSTPWTFGGGTKLEIK

SEQ ID NO: 23 - protein, 119 aa, synthetic construct
  REGION 1..119: note = Heavy chain variable region
QVQLQESGPGLVKPSQTLSLTCTVTGYSITSDYAWNWIRQFPGKGLEWMGYISYSGSFSYHPSLKSRVTISRDTSKNQFSLKLSSVTAADTAVYYCARSFSYGYGFAYWGQGTLVTVSS

SEQ ID NO: 24 - protein, 119 aa, synthetic construct
  REGION 1..119: note = Heavy chain variable region
QVQLQESGPGLVKPSQTLSLTCTVTGYSITSDYAWNWIRQFPGNGLEWIGYISYSGSFSYHPSLKSRVTISRDTSKNQFSLKLSSVTAADTAVYYCARSFSYGYGFAYWGQGTLVTVSS

SEQ ID NO: 25 - protein, 119 aa, synthetic construct
  REGION 1..119: note = Heavy chain variable region
QVQLQESGPGLVKPSQTLSLTCTVTGYSITSDYAWNWIRQFPGNGLEWMGYISYSGSFSYHPSLKSRVTISRDTSKNQFSLKLSSVTAADTAVYYCARSFSYGYGFAYWGQGTLVTVSS

SEQ ID NO: 26 - protein, 119 aa, synthetic construct
  REGION 1..119: note = Heavy chain variable region
QVQLQESGPGLVKPSQTLSLTCTVTGYSITSDYAWNWIRQFPGNKVEWMGYISYSGSFSYHPSLKSRVTISRDTSKNQFSLKLSSVTAADTAVYYCARSFSYGYGFAYWGQGTLVTVSS

SEQ ID NO: 27 - protein, 119 aa, synthetic construct
  REGION 1..119: note = Heavy chain variable region
QVQLQESGPGLVKPSQTLSLTCTVTGYSITSDYAWNWIRQFPGKGLEWMGYISYSGSFSYNPSLKSRVTISRDTSKNQFSLKLSSVTAADTAVYYCARSFSYSYGFAYWGQGTLVTVSS

SEQ ID NO: 28 - protein, 119 aa, synthetic construct
  REGION 1..119: note = Heavy chain variable region
QVQLQESGPGLVKPSQTLSLTCTVTGYSITSDYTWNWIRQFPGKGLEWMGYISYSGSFSYNPSLKSRVTISRDTSKNQFSLKLSSVTAADTAVYYCARSFSYSYGFAYWGQGTLVTVSS

SEQ ID NO: 29 - protein, 119 aa, synthetic construct
  REGION 1..119: note = Heavy chain variable region
QVQLQESGPGLVKPSQTLSLTCTVTGYSITSDYAWNWIRQFPGKGLEWMGYISYSGSYSYNPSLKSRVTISRDTSKNQFSLKLSSVTAADTAVYYCARSFSYSYGFAYWGQGTLVTVSS

SEQ ID NO: 30 - protein, 119 aa, synthetic construct
  REGION 1..119: note = Heavy chain variable region
QVQLQESGPGLVKPSQTLSLTCTVTGYSITSDYTWNWIRQFPGKGLEWMGYISYSGSYSYNPSLKSRVTISRDTSKNQFSLKLSSVTAADTAVYYCARSFSYSYGFAYWGQGTLVTVSS

SEQ ID NO: 31 - protein, 119 aa, synthetic construct
  REGION 1..119: note = Heavy chain variable region
QVQLQESGPGLVKPSQTLSLTCTVTGYSITSDYTWNWIRQFPGKGLEWMGYISYSGSYSYHPSLKSRVTISRDTSKNQFSLKLSSVTAADTAVYYCARSLSYGYGFAYWGQGTLVTVSS

SEQ ID NO: 32 - protein, 119 aa, synthetic construct
  REGION 1..119: note = Heavy chain variable region
QVQLQESGPGLVKPSQTLSLTCTVTGYSITSDYTWNWIRQFPGKGLEWMGYISYSGSFSYNPSLKSRVTISRDTSKNQFSLKLSSVTAADTAVYYCARSLSYGYGFAYWGQGTLVTVSS

SEQ ID NO: 33 - protein, 119 aa, synthetic construct
  REGION 1..119: note = Heavy chain variable region
QVQLQESGPGLVKPSQTLSLTCTVTGYSITSDYAWNWIRQFPGKGLEWMGYISYSGSYSYNPSLKSRVTISRDTSKNQFSLKLSSVTAADTAVYYCARSFSYAYGFAYWGQGTLVTVSS

SEQ ID NO: 34 - protein, 119 aa, synthetic construct
  REGION 1..119: note = Heavy chain variable region
QVQLQESGPGLVKPSQTLSLTCTVTGYSITSDYTWNWIRQFPGKGLEWMGYISYSGSYSYHPSLKSRVTISRDTSKNQFSLKLSSVTAADTAVYYCARSFSYGYGFAYWGQGTLVTVSS

SEQ ID NO: 35 - protein, 448 aa, synthetic construct
  REGION 1..448: note = Heavy chain
DVQLQESGPGLVKPSQSLSLTCTVTGYSITSDYAWNWIRQFPGNKVEWMGYISYSGSFSYHPSLKSRISITRDTSKNQFFLQLSSVTTEDTATYYCARSFSYGYGFAYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 36 - protein, 214 aa, synthetic construct
  REGION 1..214: note = Light chain
DIVMTQSHKFMSTSVGDRVNITCKASQDVSSAVAWYQQKPGQSPKLLIYSASYRYTGVPDHFTGSGSGTDFTFTISSVQAEDLAVYYCQQHYSTPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 37 - protein, 448 aa, synthetic construct
  REGION 1..448: note = Heavy chain
QVQLQESGPGLVKPSQTLSLTCTVTGYSITSDYTWNWIRQFPGKGLEWMGYISYSGSYSYNPSLKSRVTISRDTSKNQFSLKLSSVTAADTAVYYCARSFSYSYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 38 - protein, 214 aa, synthetic construct
  REGION 1..214: note = Light chain
DIQMTQSPSSLSASVGDRVTITCKASQDVSSAVAWYQQKPGKAPKLLIYSASYRYTGVPSRFSGSGSGTDFTFTISSLQPEDFATYYCQQHYSTPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39-63 - skipped (25 entries)

SEQ ID NO: 64 - protein, 25 aa, synthetic construct
  REGION 1..25: note = VH FR1
QVQLQESGPGLVKPSQTLSLTCTVT

SEQ ID NO: 65 - protein, 25 aa, synthetic construct
  REGION 1..25: note = VH FR1
DVQLQESGPGLVKPSQSLSLTCTVT

SEQ ID NO: 66 - protein, 14 aa, synthetic construct
  REGION 1..14: note = VH FR2
WIRQFPGKGLEWMG

SEQ ID NO: 67 - protein, 14 aa, synthetic construct
  REGION 1..14: note = VH FR2
WIRQFPGNGLEWIG

SEQ ID NO: 68 - protein, 14 aa, synthetic construct
  REGION 1..14: note = VH FR2
WIRQFPGNGLEWMG

SEQ ID NO: 69 - protein, 14 aa, synthetic construct
  REGION 1..14: note = VH FR2
WIRQFPGNKVEWMG

SEQ ID NO: 70 - protein, 32 aa, synthetic construct
  REGION 1..32: note = VH FR3
RVTISRDTSKNQFSLKLSSVTAADTAVYYCAR

SEQ ID NO: 71 - protein, 11 aa, synthetic construct
  REGION 1..11: note = VH FR4
WGQGTLVTVSS

SEQ ID NO: 72 - protein, 32 aa, synthetic construct
  REGION 1..32: note = VH FR3
RISITRDTSKNQFFLQLSSVTTEDTATYYCAR

SEQ ID NO: 73 - protein, 15 aa, synthetic construct
  REGION 1..15: note = VL FR2
WYQQKPGQSPKLLIY

SEQ ID NO: 74 - protein, 32 aa, synthetic construct
  REGION 1..32: note = VL FR3
GVPDHFTGSGSGTDFTFTISSVQAEDLAVYYC

SEQ ID NO: 75 - protein, 10 aa, synthetic construct
  REGION 1..10: note = VL FR4
FGGGTKLEIK

SEQ ID NO: 76 - protein, 23 aa, synthetic construct
  REGION 1..23: note = VL FR1
DIQMTQSPSSLSASVGDRVTITC

SEQ ID NO: 77 - protein, 15 aa, synthetic construct
  REGION 1..15: note = VL FR2
WYQQKPGKAPKLLIY

SEQ ID NO: 78 - protein, 32 aa, synthetic construct
  REGION 1..32: note = VL FR3
GVPSRFSGSGSGTDFTFTISSLQPEDFATYYC

SEQ ID NO: 79 - protein, 10 aa, synthetic construct
  REGION 1..10: note = VL FR4
FGGGTKVEIK

SEQ ID NO: 80 - protein, 23 aa, synthetic construct
  REGION 1..23: note = VL FR1
DIVMTQSPSSLSASVGDRVTITC

SEQ ID NO: 81 - protein, 32 aa, synthetic construct
  REGION 1..32: note = VL FR3
GVPDRFSGSGSGTDFTFTISSLQPEDFATYYC

SEQ ID NO: 82 - protein, 23 aa, synthetic construct
  REGION 1..23: note = VL FR1
DIVMTQSHKFMSTSVGDRVNITC

SEQ ID NO: 83 - protein, 10 aa, synthetic construct
  REGION 1..10: note = CDRH3
SFSYAYGFAY

SEQ ID NO: 84 - protein, 448 aa, synthetic construct
QVQLQESGPGLVKPSQTLSLTCTVTGYSITSDYTWNWIRQFPGKGLEWMGYISYSGSYSYNPSLKSRVTISRDTSKNQFSLKLSSVTAADTAVYYCARSFSYSYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2026/0234245 A1
(43) Pub. Date: Aug. 13, 2026

(54) NOVEL BINDING MOLECULES BINDING TO L1CAM

(71) Applicants: ELTHERA AG, Zug (CH); DEUTSCHES KREBSFORSCHUNGSZENTRUM STIFTUNG DES ÖFFENTLICHEN RECHTS, Heidelberg (DE)

(72) Inventors: Gunther SPOHN, Weiningen (CH); Annette SCHMIDT, Zürich (CH); Flavio MEHLI, Horgen (CH); Peter ALTEVOGT, Neckargemünd (DE); Sandra LÜTTGAU, Schongau (DE); Gerhard MOLDENHAUER, Marburg (DE)

(73) Assignees: ELTHERA AG, Zug (CH); DEUTSCHES KREBSFORSCHUNGSZENTRUM STIFTUNG DES ÖFFENTLICHEN RECHTS, Heidelberg (DE)

(21) Appl. No.: 19/113,378

(22) PCT Filed: Sep. 21, 2023
(86) PCT No.: PCT/EP2023/076077

§ 371 Date: Mar. 19, 2025

(30) Foreign Application Priority Data
Sep. 21, 2022 (EP) 22196917.3

(51) Int. Cl. C07K 16/28; A61K 39/00; A61K 47/68; A61P 35/00

(52) U.S. Cl. CPC C07K 16/2803; A61K 47/68031; A61K 47/68033; A61K 47/68035; A61K 47/6849; A61P 35/00; A61K 2039/505; C07K 2317/24; C07K 2317/33; C07K 2317/35; C07K 2317/565; C07K 2317/622; C07K 2317/732; C07K 2317/92; C07K 2317/94

(57) ABSTRACT

The present invention relates to an antibody that specifically binds to human L1CAM as specified in the claims, related nucleic acids, host cells and pharmaceutical compositions and related methods and uses.

### CROSS REFERENCE TO RELATED APPLICATIONS

This application is a U.S. National Phase application under 35 U.S.C. § 371 of International Application No. PCT/EP2023/076077, filed Sep. 21, 2023, designating the United States and published in English, which claims priority to and the benefit of European Application No. 22196917.3 filed Sep. 21, 2022, the entire contents of each of which are incorporated by reference herein.

### SEQUENCE LISTING

This application contains a Sequence Listing which has been submitted electronically in XML format and is hereby incorporated by reference in its entirety. The Sequence Listing XML file, created on Feb. 20, 2026, is named “202402-020302_US SL.xml” and is 82,310 bytes in size.

The present invention relates to an antibody that specifically binds to human L1CAM as specified in the claims, related nucleic acids, host cells, pharmaceutical compositions as well as related methods and uses.

### BACKGROUND

Monoclonal antibodies (mAbs) have emerged as a new and important pillar for cancer therapy [1]. During the past two decades molecular biology has provided means to create chimeric, humanized or fully human antibodies for the treatment of major malignant diseases [2]. To date, many antibodies and antibody-conjugates are approved as cancer therapeutics for marketing in Europe and the United States [3,4]. They comprise unmodified antibodies, antibody-drug conjugates as well as conjugates with radionuclides and a bispecific antibody [5]. However, it is quite known that mAbs to a given cancer antigen may differ in their ability to target the cancer cell.

In recent work it was shown that L1CAM (Neural cell adhesion molecule L1, also called L1) may be an excellent target molecule for human cancers. L1CAM is a transmembrane glycoprotein usually involved in the development of the nervous system by influencing cell adhesion and cell motility. L1CAM is overexpressed in many human cancers, confers bad prognosis and augments cell motility, invasion and metastasis.

WO 2008/151819 discloses the anti-L1CAM antibody L1CAM mAb L1-9.3 (also called mAb 9.3 or L9.3), which binds to an epitope within the first Ig domain of L1CAM. Results from xenograft [6] and human L1CAM transgenic mouse [7] models have suggested that mAb 9.3 might be a promising tool for cancer therapy. Recent results have shown that this mAb in its IgG2a version is well suited to activate the immune system and recruit immune effector cells leading to the elimination of cancer cells [6, 7].

WO 2016/050702 discloses binding molecules binding to L1CAM, which are capable of binding to the same L1CAM epitope recognized by the monoclonal antibody L1-OV52.24, and/or which compete with the monoclonal antibody L1-OV52.24 for binding to L1CAM. The binding molecules of WO 2016/050702 have been shown to have an improved internalization rate in comparison to mAb 9.3. It has been described that binding of L1CAM specific antibodies will lead to L1CAM internalization followed by recycling or degradation of the target molecule [8]. Internalization is a feature that L1CAM has in common with many other cell surface molecules. In fact, it has been described that L1CAM internalization is required for signaling and regulation of L1CAM mediated cell adhesion [9-11].

Despite these discoveries, there is an ongoing need for antibodies which exhibit specific binding with high affinity to L1CAM on the surface of human cancer cell lines, but not showing any reactivity with plate-coated human CHL1, NrCAM or Neurofascin. Further, there is a need for antibodies inhibiting migration and proliferation of cancer cells, inducing cytolysis of tumor cells, inhibiting metastasis formation and reducing tumor load as well as the development of ascites fluid. Also, it would be advantageous to provide antibodies improving general clinical signs of disease. Finally, it would generally be helpful, if such antibodies at the same time have a high conformational and chemical stability and show reduced formation of posttranslational variants.

Even though some features of the antibody of the invention have in part already been described, c.f. [6], the antibody itself or the sequence of the complementarity determining regions (CDR) of the antibody of the invention has never been published or made available to the public.

The antibodies of the present invention that specifically bind to human L1CAM solve all above-mentioned problems and are surprisingly advantageous in the field of biotechnological research, diagnosis or therapy.

### SUMMARY OF THE INVENTION

An antibody that specifically binds to human L1CAM is provided.

Moreover, a nucleic acid coding for a whole or parts of an antibody that specifically binds to human L1CAM is provided as well as a host cell comprising such a nucleic acid.

Also provided is a pharmaceutical composition comprising such antibody, nucleic acid or host cell.

Such antibody, nucleic acid, host cell or pharmaceutical composition for use as a medicament or as a diagnostic agent or for use in treating or preventing a hyperproliferative disorder, a tumor disease, a disorder associated with neoangiogenesis and/or a disorder associated with aberrant neurogenesis is also provided.

### THE PRESENT INVENTION

The present invention provides a novel antibody that specifically binds to human L1CAM.

The novel antibody that specifically binds to human L1CAM exhibits advantageous features as compared to anti-L1CAM antibodies in the prior art.

For example, the chimeric OV549.20 human IgG1 antibody of the present invention induced robust ADCC on Panc-1 target cells. In contrast thereto, the chimeric human IgG1 version of the previously described antibody L9.3 binding to the first Ig domain of L1CAM did not induce any ADCC on Panc-1 target cells (see Example 2, FIG. 4). Further, the addition of the antibody of the present invention leads to a reduction in the proliferation of all three cancer cell lines tested (see Example 2, FIG. 5). In contrast thereto, the binding molecules of WO 2016/050702 binding to L1CAM had no influence on the proliferation of cancer cell lines (see Example 2, FIG. 5). Furthermore, the antibody of the present invention reduced tumor mass and ascites volume in a xenograft model of human SKOV3 ovarian cancer cells in mice, while the previously described antibody L9.3 had no effect (see Example 3, FIG. 7A).

The antibodies of the invention exhibit specific binding with high binding affinity to L1CAM on the surface of human cancer cell lines, but do not show any reactivity with plate-coated human CHL1, NrCAM or Neurofascin. Further, antibodies of the invention inhibit migration and proliferation of a variety of different cancer cells, induce cytolysis of tumor cells, inhibit metastasis formation and reduce tumor load as well as the development of ascites fluid. This combination of properties make the antibody particularly suitable for improving clinical signs of tumor disease in general. In addition, the antibodies of the invention at the same time have a high conformational and chemical stability and show reduced formation of posttranslational variants, which make them particularly suitable for large scale manufacturing, clinical development, clinical safety and storage.

The term “antibody that specifically binds to human L1CAM”, as used herein, means any polypeptide, which has structural similarity to a naturally occurring antibody and is capable of binding to human L1CAM, wherein the binding specificity is determined by the CDRs of the polypeptide. Hence, “antibody that specifically binds to human L1CAM” is intended to relate to an immunoglobulin-derived structure with binding to human L1CAM.

L1CAM (also called L1), is a transmembrane protein; it is a neuronal cell adhesion molecule, member of the L1 protein family, of 200-220 kDa, and involved in axon guidance and cell migration with a strong implication in treatment-resistant cancers. The term “human L1CAM” according to the present invention is preferably understood as human L1CAM protein. The human L1CAM gene sequence has been assigned Gene ID: 3897. The Genbank entry for the isoform 1 precursor of human L1CAM protein is NP_000416. L1CAM has also been designated CD171. The term “human L1CAM” describes any form of protein known to be expressed based on this L1CAM gene by any cell type of a human being.

“Specific binding” is understood that the binding of the binding molecule to L1CAM is at least 50-fold, preferably at least 100-fold stronger than the binding to a control protein such as albumin, as determined e.g. by methods known to the person skilled in the art, such as surface plasmon resonance-based kinetic binding analyses. Alternatively, also methods such as Western Blot analysis, Enzyme-linked Immunosorbent Assay (ELISA) or determining shifts in the fluorescent signal in cytometer-based assays may be used. Such specific binding may be based on any interaction between an antibody and its antigen known to the person skilled in the art, such as non-covalent bonds (e.g. van der Waals contacts, hydrogen-bond formation or hydrophobic interactions).

The term “antibody” generally describes any polypeptide having structural similarity to a naturally occurring antibody, such as a protein belonging to the protein family of immunoglobulins. The term “antibody” includes full length antibodies, antigen-binding fragments of antibodies, and molecules comprising antibody VH regions and/or VL regions. Antibodies include monoclonal antibodies, recombinantly produced antibodies, monospecific antibodies, multispecific antibodies, including bispecific antibodies, human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, single domain antibodies, monovalent antibodies, single chain antibodies or single-chain Fvs (scFv), camelid antibodies, Fab fragments, F(ab′)2 fragments, disulfide-linked Fvs (dsFv), anti-idiotypic (anti-Id) antibodies, and antigen-binding fragments of any of the above. The antibody may be part of fusion proteins or conjugates. For example, an antibody may be comprised in a Chimeric Antigen Receptor (CAR). Antibodies can be of any type (e.g. IgG, IgE, IgM, IgD, IgA or IgY), any isotype (e.g. IgG1, IgG2, IgG3, IgG4, IgA1 or IgA2), or any subisotype (e.g., IgG2a or IgG2b) of immunoglobulin molecule. Each heavy and each light chain may have a variable and a constant region or parts thereof. In case the antibody contains a heavy chain constant region or parts thereof, the constant region of a heavy chain may be one of five types of mammalian Ig heavy chains: α, δ, ε, γ and μ. The type of the heavy chain present usually defines the class (isotype) of the antibody: IgA, IgD, IgE, IgG and IgM antibodies, respectively. Similarly, the constant region of a light chain may be one of two types of mammalian Ig light chains: K and A. The variable regions of heavy and light chains are usually made of a unique combination of numerous protein sequences allowing the binding to a particular antigen. The term “antibody” further includes domain-scaffolds such as affibodies, anticalins, affilins, atrimers, DARPins, FN3 scaffolds, such as adnectins and centyrins, fynomers, Kunitz domains, pronectins and OBodies.

Preferably, the antibodies described herein may be IgG antibodies, or an isotype thereof, such as human IgG1, human IgG2 or human IgG4.

Preferably, the antibody is a humanized monoclonal antibody. Alternatively, the antibody may be a chimeric antibody. Alternatively, the antibody may be a human antibody. For example, an antibody described herein is an IgG1 or IgG2 antibody.

In general, in cases of full-length, intact antibodies, each heavy chain is connected to one of the light chains, whereby the variable regions of a heavy and a light chain combine to form one of the two identical antigen-binding sites and their constant regions combine to form the constant region of the antibody. Further, both constructs of one heavy and one light chain may be connected via the constant regions of their heavy chains, forming a “Y”-shaped molecule, whereby the two arms depict the antigen-binding variable region and the stem depicts the constant region.

The antibody according to any of the aspects of the invention herein may be an intact antibody, meaning that it usually comprises a heavy chain of three or four constant domains and a light chain of one constant domain as well as the respective variable domains, whereby each domain may comprise further modifications, such as mutations, deletions or insertions, which do not change the overall domain structure.

In general, each heavy chain variable region and each light chain variable region of an antibody comprises three non-consecutively arranged complementary-determining regions (CDRs).

As used herein, the term “CDR” or “complementarity determining region” means the noncontiguous antigen combining sites found within the variable region of both heavy and light chain polypeptides. These particular regions have been described by Kabat et al., J. Biol. Chem. 252, 6609-6616 (1977) ([12]) and Kabat et al., Sequences of protein of immunological interest. (1991), and by Chothia et al., J. Mol. Biol. 196:901-917 (1987) and by MacCallum et al., J. Mol. Biol. 262:732-745 (1996) ([13]-[15]) where the definitions include overlapping or subsets of amino acid residues when compared against each other. The amino acid residues which encompass the CDRs as defined by each of the above cited references are set forth for comparison. Preferably, the term “CDR” is a CDR as defined by Kabat, based on sequence comparisons.

CDRs usually are numbered CDR1, CDR2 and CDR3 for the heavy chain variable region and the light chain variable region, respectively. As a result, an arm of an antibody usually has 6 CDRs, which together form an antigen-binding site. In general, CDRs usually each are 1 to 25 amino acids in length, preferably 3 to 20 amino acids in length, such as 3 to 16 amino acids in length. An antibody may comprise one, two or more arms, i.e. one, two or three antigen-binding sites.

In an aspect, the present invention relates to an antibody that specifically binds to human L1CAM, comprising:
(a) a heavy chain variable region (VH) complementarity determining region (CDR) 1 comprising the amino acid sequence of GYSITSDYX₁WN (SEQ ID NO: 16), wherein:
   X₁ is A or T;
   or a sequence containing 1, 2 or 3 amino acid exchanges in the sequence of (SEQ ID NO: 16);
(b) a VH CDR2 comprising the amino acid sequence of YISYSGSX₁SYX₂PSLKS (SEQ ID NO: 17), wherein
   X₁ is F or Y, and
   X₂ is H or N;
   or a sequence containing 1, 2 or 3 amino acid exchanges in the sequence of (SEQ ID NO: 17);
(c) a VH CDR3 comprising the amino acid sequence of SX₁SYX₂YGFAY (SEQ ID NO: 18), wherein:
   X₁ is L or F, and
   X₂ is G, S or A;
   or a sequence containing 1, 2 or 3 amino acid exchanges in the sequence of (SEQ ID NO: 18);
(d) a light chain variable region (VL) CDR1 comprising the amino acid sequence of KASQDVSSAVA (SEQ ID NO: 4);
   or a sequence containing 1, 2 or 3 amino acid exchanges in the sequence of (SEQ ID NO: 4);
(e) a VL CDR2 comprising the amino acid sequence of SASYRYX₁ (SEQ ID NO: 19), wherein:
   X₁ is T or I;
   or a sequence containing 1, 2 or 3 amino acid exchanges in the sequence of (SEQ ID NO: 19); and
(f) a VL CDR3 comprising the amino acid sequence of QQHYSTPWT (SEQ ID NO: 6);
   or a sequence containing 1, 2 or 3 amino acid exchanges in the sequence of (SEQ ID NO: 6).

In an aspect, the present invention relates to an antibody that specifically binds to human L1CAM, comprising:
(a) a heavy chain variable region (VH) complementarity determining region (CDR) 1 comprising the amino acid sequence of GYSITSDYX₁WN (SEQ ID NO: 16), wherein:
   X₁ is A or T;
(b) a VH CDR2 comprising the amino acid sequence of YISYSGSX₁SYX₂PSLKS (SEQ ID NO: 17), wherein
   X₁ is F or Y, and
   X₂ is H or N;
(c) a VH CDR3 comprising the amino acid sequence of SX₁SYX₂YGFAY (SEQ ID NO: 18), wherein:
   X₁ is L or F, and
   X₂ is G, S or A;
(d) a light chain variable region (VL) CDR1 comprising the amino acid sequence of KASQDVSSAVA (SEQ ID NO: 4);
(e) a VL CDR2 comprising the amino acid sequence of SASYRYX₁ (SEQ ID NO: 19), wherein:
   X₁ is T or I; and
(f) a VL CDR3 comprising the amino acid sequence of QQHYSTPWT (SEQ ID NO: 6).

In this respect, a variable “X₁” or “X₂” for a given SEQ ID NO: is independent from “X₁” or “X₂” of a different SEQ ID NO:. “X₁” or “X₂” is defined independently for each SEQ ID NO:. In case a sequence contains “X₁” and “X₂”, “X₁” and “X₂” are selected independently from each other. For example, X₁ and X₂ of SEQ ID NO: 17 (VH CDR2) are usually each selected independently from each other and independently from the mentioned variants of X₁ of the sequences SEQ ID NO: 16 (VH CDR1), SEQ ID NO: 18 (VH CDR3) and SEQ ID NO: 19 (VL CDR2) and X₂ of SEQ ID NO: 18 (VH CDR3). For example, X₁ and X₂ of SEQ ID NO: 18 (VH CDR3) are usually each selected independently from each other and independently from the mentioned variants of X₁ of the sequences SEQ ID NO: 16 (VH CDR1), SEQ ID NO: 17 (VH CDR2) and SEQ ID NO: 19 (VL CDR2) and X₂ of SEQ ID NO: 17 (VH CDR2).

Finally, for example, X₁ of SEQ ID NO: 19 (VL CDR2) is usually selected independently of the mentioned variants of X₁ of the sequences SEQ ID NO: 16 (VH CDR1), SEQ ID NO: 17 (VH CDR2) and SEQ ID NO: 18 (VH CDR3) and the mentioned variants of X₂ of the sequences SEQ ID NO: 17 (VH CDR2) and SEQ ID NO: 18 (VH CDR3).

The remaining regions of the antibody according to the invention, such as the framework regions of the variable heavy and light chain, as well as, where applicable, the constant domain of the heavy chain and the constant domain of the light chain may be of any sequence, e.g. of a sequence known to the person skilled in the art.

The antibody of the invention may further comprise two identical heavy chains and/or two identical light chains or only comprise non-identical heavy and light chains. It is further possible that the heavy chain variable region and the light chain variable region comprising above-mentioned specific sets of CDR1 to CDR3, respectively, only form one arm of the antibody and a second (or yet further) arm of an antibody comprises different heavy and light chain variable regions.

As the antibody according to the invention may comprise two or more heavy chain variable regions and two or more light chain variable regions, it is further also not necessary that the heavy chain variable region comprising the specific heavy chain CDR1 to CDR3 mentioned above is connected to the light chain variable region comprising the specific light chain CDR1 to CDR3 mentioned above.

The antibody of the invention may originate from a mammal, such as from a rodent, e.g. from a mouse, a rabbit or a rat. Each remaining region of the antibody according to the invention, such as the framework regions as well as, where applicable, the constant domain of the heavy chain and the constant domain of the light chain, may comprise one or more modifications, such as mutations, including substitutions, deletions or insertions, which do not change the overall domain structure.

For example, the antibody of the invention may comprise 1 to 10 mutations (including 1, 2, 3, 4, 5, 6, 7, 8, 9 or 10 mutations, including any subrange thereof), such as substitutions, deletions and/or insertions, in particular 1 to 10 substitutions (including 1, 2, 3, 4, 5, 6, 7, 8, 9 or 10 substitutions, including any subrange thereof), in the Fc domain of an antibody. Each mutation, independently, may be introduced in one chain of the Fc domain. Alternatively, each mutation, independently, may be introduced symmetrically in both chains of the Fc domain.

For example, the antibody of the invention may comprise 1 to 10 mutations (including 1, 2, 3, 4, 5, 6, 7, 8, 9 or 10 mutations, including any subrange thereof), such as substitutions, deletions and/or insertions, in particular 1 to 10 substitutions (including 1, 2, 3, 4, 5, 6, 7, 8, 9 or 10 substitutions, including any subrange thereof), in an CL and/or CH1 domain of an antibody.

Exemplary combinations of above-mentioned VH CDR1, VH CDR2 and VH CDR3 are listed in Table 1 below in the context of various antibody examples, which all correspond to an antibody according to the invention. Therein, antibody OV549.20 represents a murine example of an antibody according to the invention. H1, H2, H3 and H4 are examples of VH regions of humanized variants of the murine antibody OV549.20. AFF1 to AFF10 are examples of antibodies according to the invention, which have been further subject to affinity maturation. Of these, the combination of CDRH1, CDRH2 and CDRH3 of the antibody “AFF4” is especially preferred. Sequences highlighted in bold are especially preferred sequences.

TABLE 1
Heavy chain CDR sequences of exemplary anti-L1CAM antibodies ¹

| | CDRH1 (SEQ ID | | |
|---|---|---|---|
| Antibody | NO:) | CDRH2 (SEQ ID NO:) | CDRH3 (SEQ ID NO:) |
| OV549.20 | GYSITSDYAWN | YISYSGSFSYHPSLKS | SFSYGYGFAY (3) |
| (murine) | (1) | (2) | |
| H1, H2, H3, | GYSITSDYAWN | YISYSGSFSYHPSLKS | SFSYGYGFAY (3) |
| H4 (heavy | (1) | (2) | |
| chain | | | |
| variable | | | |
| regions) | | | |
| AFF1, AFF3, | GYSITSDYAWN | | |
| AFF7, AFF9 | (1) | | |
| AFF2, AFF4, | GYSITSDYTWN | | |
| AFF5, AFF6, | (9) | | |
| AFF8, AFF10 | | | |
| AFF1, AFF2, | | YISYSGSFSYNPSLKS | |
| AFF6, AFF7, | | (10) | |
| AFF8 | | | |
| AFF3, AFF4, | | YISYSGSYSYNPSLKS | |
| AFF9 | | (11) | |
| AFF5, AFF 10 | | YISYSGSYSYHPSLKS | |
| | | (12) | |
| AFF5, AFF6 | | | SLSYGYGFAY (13) |
| AFF10 | | | SFSYGYGFAY (3) |
| AFF9 | | | SFSYAYGFAY (83) |
| AFF1, AFF2, | | | SFSYSYGFAY (14) |
| AFF3, AFF4, | | | |
| AFF7, AFF8 | | | |
| AFF4 | GYSITSDYTWN | YISYSGSYSYNPSLKS | SFSYSYGFAY (14) |
| | (9) | (11) | |
| Humanized | GYSITSDYTWN | YISYSGSYSYNPSLKS | SFSYSYGFAY (14) |
| optimized | (9) | (11) | |
| antibody (as | | | |
| human IgG1) | | | |
| Consensus | GYSITSDYX₁WN | YISYSGSX₁SYX₂PSLKS | SX₁SYX₂YGFAY (18) |
| sequences | (16) | (17) | X₁ is L or F, |
| | X₁ is A or T | X₁ is F or Y | X₂ is G, S or A |
| | Preferably, | X₂ is H or N | Preferably, |
| | X₁ is T | Preferably, | X₁ is F, and/or |
| | | X₁ is Y, and/or | X₂ is S |
| | | X₂ is N | |

¹ The CDRs in Table 1 are determined according to Kabat.

Exemplary combinations of above-mentioned VL CDR1, VL CDR2 and VL CDR3 of the present invention are listed in Table 2 below in the context of the various antibody examples already mentioned in Table 1. L1 and L2 are examples of light chain variable regions of humanized antibodies related to the murine antibody OV549.20, which may be further combined with any of the sets of VH CDR1, VH CDR2 and VH CDR3 of H1, H2, H3 and H4 of Table 1. The antibody may comprise the VH CDRs of one antibody as depicted in Table 1 and the VL CDRs of the same or a different antibody as depicted in Table 2, preferably the VL CDRs of the same as depicted in Table 2. Sequences highlighted in bold are especially preferred sequences. AFF1 to AFF10 are examples of antibodies according to the invention, which have been further subject to affinity maturation. Of these, the combination of CDRL1, CDRL2 and CDRL3 of antibody “AFF4” is especially preferred.

TABLE 2
Light chain CDR sequences of exemplary anti-L1CAM antibodies ²

| | CDRL1 (SEQ ID | | CDRL3 (SEQ ID |
|---|---|---|---|
| Antibody | NO:) | CDRL2 (SEQ ID NO:) | NO:) |
| OV549.20 | KASQDVSSAVA | SASYRYT (5) | QQHYSTPWT (6) |
| (murine) | (4) | | |
| L1, L2 (light | KASQDVSSAVA | SASYRYT (5) | QQHYSTPWT (6) |
| chain | (4) | | |
| variable | | | |
| regions) | | | |
| [combined | | | |
| with H1 to H4 | | | |
| VH regions] | | | |
| AFF1, AFF2, | KASQDVSSAVA | | QQHYSTPWT (6) |
| AFF3, | (4) | | |
| AFF4, AFF5, | | | |
| AFF6, | | | |
| AFF7, AFF8, | | | |
| AFF9, AFF 10 | | | |
| AFF1, AFF2, | | SASYRYT (5) | |
| AFF3, | | | |
| AFF4, AFF5, | | | |
| AFF6, | | | |
| AFF9, AFF10 | | | |
| AFF7, AFF8 | | SASYRYI (15) | |
| AFF4 | KASQDVSSAVA | SASYRYT (5) | QQHYSTPWT (6) |
| | (4) | | |
| Humanized | KASQDVSSAVA | SASYRYT (5) | QQHYSTPWT (6) |
| optimized | (4) | | |
| antibody (as | | | |
| human IgG) | | | |
| Consensus | KASQDVSSAVA | SASYRYX1 (19) | QQHYSTPWT (6) |
| sequence | (4) | X1 is T or I | |
| | | Preferably, X1 is T | |

² The CDRs in Table 2 are determined according to Kabat.

Preferably, X₁ of SEQ ID NO: 16 (VH CDR1) is T; and/or, X₁ of SEQ ID NO: 17 (VH CDR2) is Y and/or X₂ of SEQ ID NO: 17 (VH CDR2) is N; and/or X₁ of SEQ ID NO: 18 (VH CDR3) is F and/or X₂ of SEQ ID NO: 18 (VH CDR3) is S; and/or X₁ of SEQ ID NO: 19 (VL CDR1) is T.

In a preferred embodiment, the present invention relates to an antibody that specifically binds to human L1CAM of the invention, wherein:
(a) the VH CDR1 comprises the amino acid sequence of GYSITSDYTWN (SEQ ID NO: 9) or a sequence containing 1, 2 or 3 amino acid exchanges in the sequence of (SEQ ID NO: 9); and/or
(b) the VH CDR2 comprises the amino acid sequence of YISYSGSX₁SYX₂PSLKS (SEQ ID NO: 17), wherein
   X₁ is Y, and/or
   X₂ is N;
   or a sequence containing 1, 2 or 3 amino acid exchanges in the sequence of (SEQ ID NO: 17);
   and/or
(c) the VH CDR3 comprises the amino acid sequence of SX₁SYX₂YGFAY (SEQ ID NO: 18), wherein:
   X₁ is F, and/or
   X₂ is S;
   or a sequence containing 1, 2 or 3 amino acid exchanges in the sequence of (SEQ ID NO: 18);
   and/or
(d) the VL CDR2 comprises the amino acid sequence of SASYRYT (SEQ ID NO: 5);
   or a sequence containing 1, 2 or 3 amino acid exchanges in the sequence of (SEQ ID NO: 5).

In a preferred embodiment, the present invention relates to an antibody that specifically binds to human L1CAM of the invention, wherein:
(a) the VH CDR1 comprises the amino acid sequence of GYSITSDYTWN (SEQ ID NO: 9); and/or
(b) the VH CDR2 comprises the amino acid sequence of YISYSGSX₁SYX₂PSLKS (SEQ ID NO: 17), wherein
   X₁ is Y, and/or
   X₂ is N;
   and/or
(c) the VH CDR3 comprises the amino acid sequence of SX₁SYX₂YGFAY (SEQ ID NO: 18), wherein:
   X₁ is F, and/or
   X₂ is S;
   and/or
(d) the VL CDR2 comprises the amino acid sequence of SASYRYT (SEQ ID NO: 5).

In a more preferred embodiment, the antibody that specifically binds to human L1CAM of the invention described herein comprises:
(a) a heavy chain variable region (VH) comprising a VH CDR1 comprising the amino acid sequence of GYSITSDYTWN (SEQ ID NO: 9), or a sequence containing 1, 2 or 3 amino acid exchanges in the sequence of (SEQ ID NO: 9), a VH CDR2 comprising the amino acid sequence of YISYSGSYSYNPSLKS (SEQ ID NO: 11), or a sequence containing 1, 2 or 3 amino acid exchanges in the sequence of (SEQ ID NO: 11), and a VH CDR3 comprising the amino acid sequence of SFSYSYGFAY (SEQ ID NO: 14) or a sequence containing 1, 2 or 3 amino acid exchanges in the sequence of (SEQ ID NO: 14); and
(b) a light chain variable region (VL) comprising a VL CDR1 comprising the amino acid sequence of KASQDVSSAVA (SEQ ID NO: 4), or a sequence containing 1, 2 or 3 amino acid exchanges in the sequence of (SEQ ID NO: 4), a VL CDR2 comprising the amino acid sequence of SASYRYT (SEQ ID NO: 5) or a sequence containing 1, 2 or 3 amino acid exchanges in the sequence of (SEQ ID NO: 5), and a VL CDR3 comprising the amino acid sequence of QQHYSTPWT (SEQ ID NO: 6) or a sequence containing 1, 2 or 3 amino acid exchanges in the sequence of (SEQ ID NO: 6).

In a more preferred embodiment, the antibody that specifically binds to human L1CAM of the invention described herein comprises:
(a) a heavy chain variable region (VH) comprising a VH CDR1 comprising the amino acid sequence of GYSITSDYTWN (SEQ ID NO: 9), a VH CDR2 comprising the amino acid sequence of YISYSGSYSYNPSLKS (SEQ ID NO: 11), and a VH CDR3 comprising the amino acid sequence of SFSYSYGFAY (SEQ ID NO: 14); and
(b) a light chain variable region (VL) comprising a VL CDR1 comprising the amino acid sequence of KASQDVSSAVA (SEQ ID NO: 4), a VL CDR2 comprising the amino acid sequence of SASYRYT (SEQ ID NO: 5), and a VL CDR3 comprising the amino acid sequence of QQHYSTPWT (SEQ ID NO: 6).

Besides the specific CDRs mentioned above, the heavy and/or light chain variable regions of the antibody of the invention may also comprise one or more below-mentioned specific framework regions.

The antibody of the invention may comprise framework sequences from any species. Preferably, it comprises a mouse and/or human framework sequence or hybrids thereof. For example, the framework sequences may each be human, wherein optionally non-human positions are present, such as 1 to 10 (including 1, 2, 3, 4, 5, 6, 7, 8, 9, or 10 including any sub-range thereof) non-human positions.

As used herein the term “framework (FR) amino acid residues” refers to those amino acids in the framework region of an immunoglobulin chain. The term “framework region” or “FR region” as used herein, includes the amino acid residues that are part of the variable region, but are not part of the CDRs (e.g., using the Kabat definition of CDRs). The framework regions usually support the binding of the antibody to an antigen by either supporting the antibody's structure (and not being in contact with the antigen) or by directly contacting the antigen. The term “antigen” describes any molecule or molecular structure that may be bound by an antibody specific for that antigen.

Methods for producing a monoclonal antibody with the CDR sequences as mentioned above are known in the art and include the introduction of the nucleic acid sequences encoding the CDRs into suitable expression vectors encoding the desired framework sequences. Further methods are described below.

Suitable framework regions for the heavy chain variable region are e.g. SEQ ID NOs: 64-72. In detail, the heavy chain variable region of the antibody of the invention may comprise one to four framework regions selected from the group of SEQ ID NOs: 64-72. Preferably, the heavy chain variable region of the antibody of the invention comprises one, two, three or four framework regions selected from the group of SEQ ID NOs: 64-72, more preferably four framework regions selected from the group of SEQ ID NOs: 64-72.

Following the Kabat numbering system for CDRs, the VH CDRs are determined by Kabat and the framework regions are the amino acid residues surrounding the CDRs in the variable region in the format FR1, CDRH1, FR2, CDRH2, FR3, CDRH3, and FR4.

Accordingly, FR1 of the heavy chain variable region may be independently selected from SEQ ID NOs: 64 or 65, FR2 of the heavy chain variable region may be independently selected from SEQ ID NOs: 66-69, FR3 of the heavy chain variable region may be independently selected from SEQ ID NOs: 70 or 72, and/or FR4 of the heavy chain variable region may be SEQ ID NO: 71.

Suitable framework regions for the light chain variable region are e.g. SEQ ID NOs: 73-82. The light chain variable region of the antibody of the invention may comprise one to four framework regions selected from the group of SEQ ID NOs: 73-82. Preferably, the light chain variable region of the antibody of the invention comprises one, two, three or four framework regions selected from the group of SEQ ID NOs: 73-82, more preferably four framework regions selected from the group of SEQ ID NOs: 73-82.

Similarly, following the Kabat numbering system for CDRs, the VL CDRs are determined by Kabat and the framework regions are the amino acid residues surrounding the CDRs in the variable region in the format FR1, CDRL1, FR2, CDRL2, FR3, CDRL3, and FR4.

Accordingly, FR1 of the light chain variable region may be independently selected from SEQ ID NOs: 76, 80 or 82, FR2 of the light chain variable region may be independently selected from SEQ ID NOs: 73 or 77, FR3 of the light chain variable region may be independently selected from SEQ ID NOs: 74, 78 or 81, and/or FR4 of the light chain variable region may be independently selected from SEQ ID NOs: 75 or 79.

Further, FR1 to FR4 of the heavy chain variable region may be selected independently from FR1 to FR4 of the light chain variable region and FR1 to FR4 of the light chain variable region may be selected independently from FR1 to FR4 of the heavy chain variable region.

The following Tables 3 and 4 provide examples for antibodies according to the invention further comprising SEQ ID NOs: 64-72 as framework regions FR1 to FR4 of the heavy chain variable region and SEQ ID NOs: 73-82 as framework regions FR1 to FR4 of the light chain variable region, respectively. Therein, antibody OV549.20 represents a murine example of an antibody according to the invention. H1, H2, H3 and H4 are examples of VH regions of humanized variants of the murine antibody OV549.20. L1 and L2 are examples of VL regions of humanized variants of the murine antibody OV549.20. AFF1 to AFF10 are examples of antibodies according to the invention, which have been further subject to affinity maturation.

Preferably, a set of VH framework regions FR1 to FR4 is selected from the set provided for a specific antibody in Table 3 below. Preferably, a set of VL framework regions FR1 to FR4 is selected from the set provided for a specific antibody in Table 4 below. Preferably, a set of VH framework regions FR1 to FR4 is selected from the set provided for a specific antibody in Table 3 below and a set of VL framework regions FR1 to FR4 is selected from the set provided for the same specific antibody in Table 4 below.

TABLE 3
VH framework (FR) sequences of exemplary anti-L1CAM antibodies ³

| | | | | VH FR4 |
|---|---|---|---|---|
| | VH FR1 | VH FR2 | VH FR3 | (SEQ ID |
| Antibody | (SEQ ID NO:) | (SEQ ID NO:) | (SEQ ID NO:) | NO:) |
| H1-H4 | QVQLQESGPGL | | RVTISRDTSKN | |
| AFF1-AFF10 | VKPSQTLSLTCT | | QFSLKLSSVTA | |
| Humanized | VT (64) | | ADTAVYYCAR | |
| optimized | | | (70) | |
| antibody (as | | | | |
| human IgG) | | | | |
| OV549.20 | DVQLQESGPGL | | RISITRDTSKN | |
| | VKPSQSLSLTCT | | QFFLQLSSVTT | |
| | VT (65) | | EDTATYYCAR | |
| | | | (72) | |
| H1, AFF1- | | WIRQFPGKG | | |
| AFF 10 | | LEWMG (66) | | |
| Humanized | | | | |
| optimized | | | | |
| antibody (as | | | | |
| human IgG) | | | | |
| H2 | | WIRQFPGNG | | |
| | | LEWIG (67) | | |
| H3 | | WIRQFPGNG | | |
| | | LEWMG (68) | | |
| H4, OV549.20 | | WIRQFPGNK | | |
| | | VEWMG (69) | | |
| H1-H4, AFF1- | | | | WGQGTLVT |
| AFF10 | | | | VSS (71) |
| Humanized | | | | |
| optimized | | | | |
| antibody (as | | | | |
| human IgG) | | | | |
| OV549.20 | | | | |

³ The VH framework regions described in Table 3 are determined based upon the boundaries of the Kabat numbering system for CDRs. In other words, the VH CDRs are determined by Kabat and the framework regions are the amino acid residues surrounding the CDRs in the variable region in the format FR1, CDRH1, FR2, CDRH2, FR3, CDRH3, and FR4.

TABLE 4
VL framework (FR) sequences of exemplary anti-L1CAM antibodies ⁴

| | VL FR1 | VL FR2 | VL FR3 | VL FR4 |
|---|---|---|---|---|
| | (SEQ ID | (SEQ ID | (SEQ ID | (SEQ ID |
| Antibody | NO:) | NO:) | NO:) | NO:) |
| OV549.20 | DIVMTQSHKF | WYQQKPGQ | GVPDHFTGSG | FGGGTKLEIK |
| | MSTSVGDRV | SPKLLIY | SGTDFTFTISS | (75) |
| | NITC (82) | (73) | VQAEDLAVYY | |
| | | | C (74) | |
| L1 | DIQMTQSPSS | WYQQKPGK | GVPSRFSGSG | FGGGTKVEIK |
| AFF1-AFF10 | LSASVGDRVT | APKLLIY | SGTDFTFTISSL | (79) |
| Humanized | ITC (76) | (77) | QPEDFATYYC | |
| optimized | | | (78) | |
| antibody (as | | | | |
| human IgG) | | | | |
| L2 | DIVMTQSPSS | WYQQKPGQ | GVPDRFSGSG | FGGGTKLEIK |
| | LSASVGDRVT | SPKLLIY | SGTDFTFTISSL | (75) |
| | ITC (80) | (73) | QPEDFATYYC | |
| | | | (81) | |

⁴ The VL framework regions described in Table 4 are determined based upon the boundaries of the Kabat numbering system for CDRs. In other words, the VL CDRs are determined by Kabat and the framework regions are the amino acid residues surrounding the CDRs in the variable region in the format FR1, CDRL1, FR2, CDRL2, FR3, CDRL3, and FR4.

The antibody according to the invention may further comprise a heavy chain variable region sequence comprising one or more of the framework regions of the heavy chain variable region sequence of any one of SEQ ID NOs: 23-34 and/or further comprise a light chain variable region sequence comprising one or more of the framework regions of the light chain variable region sequence of any one of SEQ ID NOs: 20-22.

In a further preferred embodiment, the antibody that specifically binds to human L1CAM of the invention further comprises a heavy chain variable region sequence comprising the framework regions of the heavy chain variable region sequence of any one of SEQ ID NOs: 23-34, and/or further comprises a light chain variable region sequence comprising the framework regions of the light chain variable region sequence of any one of SEQ ID NOs: 20-22.

The antibody according to the invention may also comprise a heavy chain variable region sequence comprising an amino acid sequence selected from the group consisting of SEQ ID NOs: 23-34 and/or comprise a light chain variable region sequence comprising an amino acid sequence selected from the group consisting of SEQ ID NOs: 20-22. In this context, the heavy chain variable region may be independently selected from the selected light chain variable region and the light chain variable region may be independently selected from the selected heavy chain variable region.

Accordingly, one of the heavy chain variable regions of the antibody may be selected from the group consisting of SEQ ID NOs: 23-34, while the sequence of the second heavy chain variable region may not be selected from this group.

Also, one of the light chain variable regions of the antibody according to an embodiment may be selected from the group consisting of SEQ ID NOs: 20-22, while the sequence of the second light chain variable region may not be selected from this group.

Further, when the antibody according to the invention comprises a heavy chain variable region sequence comprising an amino acid sequence selected from the group consisting of SEQ ID NOs: 23-34 and comprises a light chain variable region sequence comprising an amino acid sequence selected from the group consisting of SEQ ID NOs: 20-22, these may not necessarily be part of the same arm of an antibody comprising two or more arms, but may also be allocated to different arms.

It is further disclosed herein for all embodiments, that the heavy chain variable region sequences, light chain variable region sequences, complementarity determining regions, light chain sequences and heavy chain sequences “comprise” or “consist” of the respective indicated sequences.

The antibody according to the invention may further comprise one or two heavy chain variable region sequence(s) comprising an amino acid sequence independently selected from the group consisting of SEQ ID NOs: 23-34 and/or comprise one or two light chain variable region sequence(s) comprising an amino acid sequence independently selected from the group consisting of SEQ ID NOs: 20-22.

In a preferred embodiment, the antibody that specifically binds to human L1CAM of the invention comprises a heavy chain variable region sequence comprising an amino acid sequence selected from the group consisting of SEQ ID NOs: 23-34, and/or comprises a light chain variable region sequence comprising an amino acid sequence selected from the group consisting of SEQ ID NOs: 20-22.

In a preferred embodiment, the antibody that specifically binds to human L1CAM of the invention comprises a heavy chain variable region sequence which has an amino acid sequence selected from the group consisting of SEQ ID NOs: 23-34, and/or comprises a light chain variable region sequence which has an amino acid sequence selected from the group consisting of SEQ ID NOs: 20-22.

Exemplary combinations of sequences of heavy and light chain variable regions are given in Table 5 below. The antibodies' names mentioned therein relate to those mentioned and explained in the context of Tables 1 to 4 above. Sequences highlighted in bold are especially preferred sequences.

Preferably, in the context of an antibody herein, a VH and/or VL sequence has a sequence as shown in Table 5 below.

TABLE 5
VH and VL sequences of antibodies⁵

| | Heavy chain | SEQ | Light chain | SEQ ID |
|---|---|---|---|---|
| Antibody | variable region | ID NO: | variable region | NO: |
| H1 | QVQLQESGPGLVKP | 23 | [combined with L1 or | |
| | SQTLSLTCTVTGYSI | | L2 in examples] | |
| | TSDYAWNWIRQFP | | | |
| | GKGLEWMGYISYSG | | | |
| | SFSYHPSLKSRVTIS | | | |
| | RDTSKNQFSLKLSS | | | |
| | VTAADTAVYYCARS | | | |
| | FSYGYGFAYWGQG | | | |
| | TLVTVSS | | | |
| H2 | QVQLQESGPGLVKP | 24 | [combined with L1 or | |
| | SQTLSLTCTVTGYSI | | L2 in examples] | |
| | TSDYAWNWIRQFP | | | |
| | GNGLEWIGYISYSG | | | |
| | SFSYHPSLKSRVTIS | | | |
| | RDTSKNQFSLKLSS | | | |
| | VTAADTAVYYCARS | | | |
| | FSYGYGFAYWGQG | | | |
| | TLVTVSS | | | |
| H3 | QVQLQESGPGLVKP | 25 | [combined with L1 or | |
| | SQTLSLTCTVTGYSI | | L2 in examples] | |
| | TSDYAWNWIRQFP | | | |
| | GNGLEWMGYISYS | | | |
| | GSFSYHPSLKSRVTI | | | |
| | SRDTSKNQFSLKLS | | | |
| | SVTAADTAVYYCAR | | | |
| | SFSYGYGFAYWGQ | | | |
| | GTLVTVSS | | | |
| H4 | QVQLQESGPGLVKP | 26 | [combined with L1 or | |
| | SQTLSLTCTVTGYSI | | L2 in examples] | |
| | TSDYAWNWIRQFP | | | |
| | GNKVEWMGYISYS | | | |
| | GSFSYHPSLKSRVTI | | | |
| | SRDTSKNQFSLKLS | | | |
| | SVTAADTAVYYCAR | | | |
| | SFSYGYGFAYWGQ | | | |
| | GTLVTVSS | | | |
| L1 | [combined with H1, | | DIQMTQSPSSLSAS | 20 |
| | H2, H3 or H4 in | | VGDRVTITCKASQD | |
| | examples] | | VSSAVAWYQQKPG | |
| | | | KAPKLLIYSASYRYT | |
| | | | GVPSRFSGSGSGT | |
| | | | DFTFTISSLQPEDFA | |
| | | | TYYCQQHYSTPWTF | |
| | | | GGGTKVEIK | |
| L2 | [combined with H1, | | DIVMTQSPSSLSAS | 22 |
| | H2, H3 or H4 in | | VGDRVTITCKASQD | |
| | examples] | | VSSAVAWYQQKPG | |
| | | | QSPKLLIYSASYRYT | |
| | | | GVPDRFSGSGSGT | |
| | | | DFTFTISSLQPEDFA | |
| | | | TYYCQQHYSTPWTF | |
| | | | GGGTKLEIK | |
| AFF1 | QVQLQESGPGLVKP | 27 | DIQMTQSPSSLSAS | 20 |
| | SQTLSLTCTVTGYSI | | VGDRVTITCKASQD | |
| | TSDYAWNWIRQFP | | VSSAVAWYQQKPG | |
| | GKGLEWMGYISYSG | | KAPKLLIYSASYRYT | |
| | SFSYNPSLKSRVTIS | | GVPSRFSGSGSGT | |
| | RDTSKNQFSLKLSS | | DFTFTISSLQPEDFA | |
| | VTAADTAVYYCARS | | TYYCQQHYSTPWTF | |
| | FSYSYGFAYWGQG | | GGGTKVEIK | |
| | TLVTVSS | | | |
| AFF2 | QVQLQESGPGLVKP | 28 | DIQMTQSPSSLSAS | 20 |
| | SQTLSLTCTVTGYSI | | VGDRVTITCKASQD | |
| | TSDYTWNWIRQFPG | | VSSAVAWYQQKPG | |
| | KGLEWMGYISYSGS | | KAPKLLIYSASYRYT | |
| | FSYNPSLKSRVTISR | | GVPSRFSGSGSGT | |
| | DTSKNQFSLKLSSV | | DFTFTISSLQPEDFA | |
| | TAADTAVYYCARSF | | TYYCQQHYSTPWTF | |
| | SYSYGFAYWGQGT | | GGGTKVEIK | |
| | LVTVSS | | | |
| AFF3 | QVQLQESGPGLVKP | 29 | DIQMTQSPSSLSAS | 20 |
| | SQTLSLTCTVTGYSI | | VGDRVTITCKASQD | |
| | TSDYAWNWIRQFP | | VSSAVAWYQQKPG | |
| | GKGLEWMGYISYSG | | KAPKLLIYSASYRYT | |
| | SYSYNPSLKSRVTIS | | GVPSRFSGSGSGT | |
| | RDTSKNQFSLKLSS | | DFTFTISSLQPEDFA | |
| | VTAADTAVYYCARS | | TYYCQQHYSTPWTF | |
| | FSYSYGFAYWGQG | | GGGTKVEIK | |
| | TLVTVSS | | | |
| AFF4 | QVQLQESGPGLVK | 30 | DIQMTQSPSSLSAS | 20 |
| | PSQTLSLTCTVTGY | | VGDRVTITCKASQD | |
| | SITSDYTWNWIRQF | | VSSAVAWYQQKPG | |
| | PGKGLEWMGYISYS | | KAPKLLIYSASYRYT | |
| | GSYSYNPSLKSRVT | | GVPSRFSGSGSGT | |
| | ISRDTSKNQFSLKL | | DFTFTISSLQPEDFA | |
| | SSVTAADTAVYYCA | | TYYCQQHYSTPWTF | |
| | RSFSYSYGFAYWG | | GGGTKVEIK | |
| | QGTLVTVSS | | | |
| AFF5 | QVQLQESGPGLVKP | 31 | DIQMTQSPSSLSAS | 20 |
| | SQTLSLTCTVTGYSI | | VGDRVTITCKASQD | |
| | TSDYTWNWIRQFPG | | VSSAVAWYQQKPG | |
| | KGLEWMGYISYSGS | | KAPKLLIYSASYRYT | |
| | YSYHPSLKSRVTISR | | GVPSRFSGSGSGT | |
| | DTSKNQFSLKLSSV | | DFTFTISSLQPEDFA | |
| | TAADTAVYYCARSL | | TYYCQQHYSTPWTF | |
| | SYGYGFAYWGQGT | | GGGTKVEIK | |
| | LVTVSS | | | |
| AFF6 | QVQLQESGPGLVKP | 32 | DIQMTQSPSSLSAS | 20 |
| | SQTLSLTCTVTGYSI | | VGDRVTITCKASQD | |
| | TSDYTWNWIRQFPG | | VSSAVAWYQQKPG | |
| | KGLEWMGYISYSGS | | KAPKLLIYSASYRYT | |
| | FSYNPSLKSRVTISR | | GVPSRFSGSGSGT | |
| | DTSKNQFSLKLSSV | | DFTFTISSLQPEDFA | |
| | TAADTAVYYCARSL | | TYYCQQHYSTPWTF | |
| | SYGYGFAYWGQGT | | GGGTKVEIK | |
| | LVTVSS | | | |
| AFF7 | QVQLQESGPGLVKP | 27 | DIQMTQSPSSLSAS | 21 |
| | SQTLSLTCTVTGYSI | | VGDRVTITCKASQD | |
| | TSDYAWNWIRQFP | | VSSAVAWYQQKPG | |
| | GKGLEWMGYISYSG | | KAPKLLIYSASYRYI | |
| | SFSYNPSLKSRVTIS | | GVPSRFSGSGSGT | |
| | RDTSKNQFSLKLSS | | DFTFTISSLQPEDFA | |
| | VTAADTAVYYCARS | | TYYCQQHYSTPWTF | |
| | FSYSYGFAYWGQG | | GGGTKVEIK | |
| | TLVTVSS | | | |
| AFF8 | QVQLQESGPGLVKP | 28 | DIQMTQSPSSLSAS | 21 |
| | SQTLSLTCTVTGYSI | | VGDRVTITCKASQD | |
| | TSDYTWNWIRQFPG | | VSSAVAWYQQKPG | |
| | KGLEWMGYISYSGS | | KAPKLLIYSASYRYI | |
| | FSYNPSLKSRVTISR | | GVPSRFSGSGSGT | |
| | DTSKNQFSLKLSSV | | DFTFTISSLQPEDFA | |
| | TAADTAVYYCARSF | | TYYCQQHYSTPWTF | |
| | SYSYGFAYWGQGT | | GGGTKVEIK | |
| | LVTVSS | | | |
| AFF9 | QVQLQESGPGLVKP | 33 | DIQMTQSPSSLSAS | 20 |
| | SQTLSLTCTVTGYSI | | VGDRVTITCKASQD | |
| | TSDYAWNWIRQFP | | VSSAVAWYQQKPG | |
| | GKGLEWMGYISYSG | | KAPKLLIYSASYRYT | |
| | SYSYNPSLKSRVTIS | | GVPSRFSGSGSGT | |
| | RDTSKNQFSLKLSS | | DFTFTISSLQPEDFA | |
| | VTAADTAVYYCARS | | TYYCQQHYSTPWTF | |
| | FSYAYGFAYWGQG | | GGGTKVEIK | |
| | TLVTVSS | | | |
| AFF10 | QVQLQESGPGLVKP | 34 | DIQMTQSPSSLSAS | 20 |
| | SQTLSLTCTVTGYSI | | VGDRVTITCKASQD | |
| | TSDYTWNWIRQFPG | | VSSAVAWYQQKPG | |
| | KGLEWMGYISYSGS | | KAPKLLIYSASYRYT | |
| | YSYHPSLKSRVTISR | | GVPSRFSGSGSGT | |
| | DTSKNQFSLKLSSV | | DFTFTISSLQPEDFA | |
| | TAADTAVYYCARSF | | TYYCQQHYSTPWTF | |
| | SYGYGFAYWGQGT | | GGGTKVEIK | |
| | LVTVSS | | | |
| Humanized | QVQLQESGPGLVK | 30 | DIQMTQSPSSLSAS | 20 |
| optimized | PSQTLSLTCTVTGY | | VGDRVTITCKASQD | |
| antibody | SITSDYTWNWIRQF | | VSSAVAWYQQKPG | |
| (as human | PGKGLEWMGYISYS | | KAPKLLIYSASYRYT | |
| IgG) | GSYSYNPSLKSRVT | | GVPSRFSGSGSGT | |
| | ISRDTSKNQFSLKL | | DFTFTISSLQPEDFA | |
| | SSVTAADTAVYYCA | | TYYCQQHYSTPWTF | |
| | RSFSYSYGFAYWG | | GGGTKVEIK | |
| | QGTLVTVSS | | | |

⁵The CDRs are shown underlined in Table 5. The CDRs in Table 5 are determined
according to Kabat.

In a further preferred embodiment, the antibody comprises at least one VH region and at least one VL region of the antibodies designated AFF1 to AFF10 herein. Accordingly, preferably, the antibody comprises at least one VH region and at least one VL region of the antibody designated AFF1 herein. Accordingly, preferably, the antibody comprises at least one VH region and at least one VL region of the antibody designated AFF2 herein. Accordingly, preferably, the antibody comprises at least one VH region and at least one VL region of the antibody designated AFF3 herein. Accordingly, preferably, the antibody comprises at least one VH region and at least one VL region of the antibody designated AFF4 herein. Accordingly, preferably, the antibody comprises at least one VH region and at least one VL region of the antibody designated AFF5 herein. Accordingly, preferably, the antibody comprises at least one VH region and at least one VL region of the antibody designated AFF6 herein. Accordingly, preferably, the antibody comprises at least one VH region and at least one VL region of the antibody designated AFF7 herein. Accordingly, preferably, the antibody comprises at least one VH region and at least one VL region of the antibody designated AFF8 herein. Accordingly, preferably, the antibody comprises at least one VH region and at least one VL region of the antibody designated AFF9 herein. Accordingly, preferably, the antibody comprises at least one VH region and at least one VL region of the antibody designated AFF10 herein. The sequences of the VH and VL regions are shown in Table 5 above.

The use of AFF4 is particularly preferred. The VH region of AFF4 has the amino acid sequence of SEQ ID NO: 30, and the VL region of AFF4 has the amino acid sequence of SEQ ID NO: 20.

Accordingly, the antibody may comprise a heavy chain variable region sequence comprising the amino acid sequence of SEQ ID NO: 30 and/or comprises a light chain variable region sequence comprising the amino acid sequence of SEQ ID NO: 20. Accordingly, the antibody may also comprise one or more heavy chain variable region sequences comprising the amino acid sequence of SEQ ID NO: 30 and/or comprise one or more light chain variable region sequence comprising the amino acid sequence of SEQ ID NO: 20.

Further, in cases where the antibody comprises a heavy chain variable region sequence comprising the amino acid sequence of SEQ ID NO: 30 and comprises a light chain variable region sequence comprising the amino acid sequence of SEQ ID NO: 20, these do not necessarily be part of the same arm of an antibody comprising two or more arms, but may also be allocated to different arms.

In a yet further preferred embodiment, the antibody comprises a heavy chain variable region sequence comprising the amino acid sequence of SEQ ID NO: 30 and/or comprises a light chain variable region sequence comprising the amino acid sequence of SEQ ID NO: 20.

Moreover, the antibody of the invention may comprise a heavy chain sequence comprising or consisting of an amino acid sequence selected from the group of SEQ ID NOs: 35, 84 and 37.

The antibody of the invention may further comprise a light chain sequence comprising or consisting of an amino acid sequence of SEQ ID NOs: 36 and 38.

In addition, the antibody of the invention may comprise one or two heavy chain sequence(s) comprising or consisting of an amino acid sequence independently selected from the group of SEQ ID NOs: 35, 84 and 37 and/or comprise one or two light chain sequence(s) comprising or consisting of an amino acid sequence independently selected from the group of SEQ ID NOs: 36 and 38.

Preferably, the antibody comprises
(a) a heavy chain sequence comprising or consisting of an amino acid sequence of SEQ ID NO: 35 and/or a light chain sequence comprising or consisting of an amino acid sequence of SEQ ID NO: 36; and/or
(b) a heavy chain sequence comprising or consisting of an amino acid sequence of SEQ ID NO: 37 and/or a light chain sequence comprising or consisting of an amino acid sequence of SEQ ID NO: 38; and/or
(c) a heavy chain sequence comprising or consisting of an amino acid sequence of SEQ ID NO: 84 and/or a light chain sequence comprising or consisting of an amino acid sequence of SEQ ID NO: 38.

In a yet further preferred embodiment, the antibody comprises a heavy chain sequence comprising or consisting of an amino acid sequence of SEQ ID NO: 37 or 84 and/or a light chain sequence comprising or consisting of an amino acid sequence of SEQ ID NO: 38. In a yet further preferred embodiment, the antibody comprises a heavy chain sequence comprising or consisting of an amino acid sequence of SEQ ID NO: 37 or 84 and a light chain sequence comprising or consisting of an amino acid sequence of SEQ ID NO: 38.

In a yet further preferred embodiment, the antibody comprises a heavy chain sequence comprising or consisting of an amino acid sequence of SEQ ID NO: 37 and/or a light chain sequence comprising or consisting of an amino acid sequence of SEQ ID NO: 38. In a yet further preferred embodiment, the antibody comprises a heavy chain sequence comprising or consisting of an amino acid sequence of SEQ ID NO: 37 and a light chain sequence comprising or consisting of an amino acid sequence of SEQ ID NO: 38.

In a yet further preferred embodiment, the antibody comprises a heavy chain sequence comprising or consisting of an amino acid sequence of SEQ ID NO: 84 and/or a light chain sequence comprising or consisting of an amino acid sequence of SEQ ID NO: 38. In a yet further preferred embodiment, the antibody comprises a heavy chain sequence comprising or consisting of an amino acid sequence of SEQ ID NO: 84 and a light chain sequence comprising or consisting of an amino acid sequence of SEQ ID NO: 38.

In another further preferred embodiment, the antibody consists of a heavy chain sequence comprising or consisting of an amino acid sequence of SEQ ID NO: 37 or 84 and/or a light chain sequence comprising or consisting of an amino acid sequence of SEQ ID NO: 38. In another further preferred embodiment, the antibody consists of a heavy chain sequence comprising or consisting of an amino acid sequence of SEQ ID NO: 37 or 84 and a light chain sequence comprising or consisting of an amino acid sequence of SEQ ID NO: 38.

In another further preferred embodiment, the antibody consists of a heavy chain sequence comprising or consisting of an amino acid sequence of SEQ ID NO: 37 and/or a light chain sequence comprising or consisting of an amino acid sequence of SEQ ID NO: 38. In another further preferred embodiment, the antibody consists of a heavy chain sequence comprising or consisting of an amino acid sequence of SEQ ID NO: 37 and a light chain sequence comprising or consisting of an amino acid sequence of SEQ ID NO: 38.

In another further preferred embodiment, the antibody consists of a heavy chain sequence comprising or consisting of an amino acid sequence of SEQ ID NO: 84 and/or a light chain sequence comprising or consisting of an amino acid sequence of SEQ ID NO: 38. In another further preferred embodiment, the antibody consists of a heavy chain sequence comprising or consisting of an amino acid sequence of SEQ ID NO: 84 and a light chain sequence comprising or consisting of an amino acid sequence of SEQ ID NO: 38.

In Example 5, humanized optimized antibodies were prepared and used. In Example 5, humanized optimized antibody “AFF4” is provided which is an IgG-type antibody wherein the heavy chain variable region sequence consists of the amino acid sequence of SEQ ID NO: 30 and the light chain variable region sequence consists of the amino acid sequence of SEQ ID NO: 20, linked to a human IgG1 heavy chain constant region with G236A/S239D/A330L/I332E mutations (EU numbering) in the human IgG1 constant region. The heavy chain of the antibody consists of the amino acid sequence of SEQ ID NO: 37 and the light chain sequence consists of the amino acid sequence of SEQ ID NO: 38.

In the Examples, also the humanized optimized antibody designated “AFF4-WT” was prepared and used. Antibody “AFF4-WT” is an IgG-type antibody wherein the heavy chain variable region sequence consists of the amino acid sequence of SEQ ID NO: 30 and the light chain variable region sequence consists of the amino acid sequence of SEQ ID NO: 20, linked to wildtype human IgG1 constant region. The heavy chain of AFF4-WT consists of the amino acid sequence of SEQ ID NO: 84 and the light chain sequence consists of the amino acid sequence of SEQ ID NO: 38.

Optionally, 1, 2 or 3 amino acids may be deleted at the C-terminus of a full-length heavy chain. It is known that such deletion(s) do not affect the stability of antibodies. Moreover, it is further possible to use a heavy chain including the C-terminal Lysine of the Fc domain. Depending on the recombinant expression system used, the C-terminal Lysine of the Fc domain may be present or absent.

Exemplary combinations of sequences of heavy and light chain variable regions are given in Table 6 below. The antibodies' names mentioned therein relate to those mentioned and explained in the context of Tables 1 to 5 above.

TABLE 6
Heavy chain (HC) and light chain (LC) sequences of exemplary anti-
L1CAM antibodies ⁶

| | | SEQ | | SEQ |
|---|---|---|---|---|
| | | ID | | ID |
| Antibody | Heavy chain | NO: | Light chain | NO: |
| OV549.20 | DVQLQESGPGLVKPSQS | 35 | DIVMTQSHKFMSTSVGDR | 36 |
| (murine) | LSLTCTVTGYSITSDYAW | | VNITCKASQDVSSAVAWY | |
| | NWIRQFPGNKVEWMGYI | | QQKPGQSPKLLIYSASYR | |
| | SYSGSFSYHPSLKSRISIT | | YTGVPDHFTGSGSGTDFT | |
| | RDTSKNQFFLQLSSVTTE | | FTISSVQAEDLAVYYCQQ | |
| | DTATYYCARSFSYGYGF | | HYSTPWTFGGGTKLEIKR | |
| | AYWGQGTLVTVSSAKTT | | ADAAPTVSIFPPSSEQLTS | |
| | APSVYPLAPVCGDTTGS | | GGASVVCFLNNFYPKDIN | |
| | SVTLGCLVKGYFPEPVTL | | VKWKIDGSERQNGVLNS | |
| | TWNSGSLSSGVHTFPAV | | WTDQDSKDSTYSMSSTLT | |
| | LQSDLYTLSSSVTVTSST | | LTKDEYERHNSYTCEATH | |
| | WPSQSITCNVAHPASST | | KTSTSPIVKSFNRNEC | |
| | KVDKKIEPRGPTIKPCPP | | | |
| | CKCPAPNLLGGPSVFIFP | | | |
| | PKIKDVLMISLSPIVTCVV | | | |
| | VDVSEDDPDVQISWFVN | | | |
| | NVEVHTAQTQTHREDYN | | | |
| | STLRVVSALPIQHQDWM | | | |
| | SGKEFKCKVNNKDLPAPI | | | |
| | ERTISKPKGSVRAPQVYV | | | |
| | LPPPEEEMTKKQVTLTC | | | |
| | MVTDFMPEDIYVEWTNN | | | |
| | GKTELNYKNTEPVLDSD | | | |
| | GSYFMYSKLRVEKKNWV | | | |
| | ERNSYSCSVVHEGLHNH | | | |
| | HTTKSFSRTPG | | | |
| Humanized | QVQLQESGPGLVKPSQT | 37 | DIQMTQSPSSLSASVGDR | 38 |
| optimized | LSLTCTVTGYSITSDYTW | | VTITCKASQDVSSAVAWY | |
| antibody | NWIRQFPGKGLEWMGYI | | QQKPGKAPKLLIYSASYRY | |
| “AFF4” | SYSGSYSYNPSLKSRVTI | | TGVPSRFSGSGSGTDFTF | |
| (human | SRDTSKNQFSLKLSSVTA | | TISSLQPEDFATYYCQQHY | |
| IgG | ADTAVYYCARSFSYSYG | | STPWTFGGGTKVEIKRTV | |
| format; with | FAYWGQGTLVTVSSAST | | AAPSVFIFPPSDEQLKSGT | |
| G236A/ | KGPSVFPLAPSSKSTSG | | ASVVCLLNNFYPREAKVQ | |
| S239D/ | GTAALGCLVKDYFPEPV | | WKVDNALQSGNSQESVT | |
| A330L/ | TVSWNSGALTSGVHTFP | | EQDSKDSTYSLSSTLTLSK | |
| 1332E | AVLQSSGLYSLSSVVTVP | | ADYEKHKVYACEVTHQGL | |
| mutations | SSSLGTQTYICNVNHKPS | | SSPVTKSFNRGEC | |
| (EL | NTKVDKRVEPKSCDKTH | | | |
| numbering)) | TCPPCPAPELLAGPDVFL | | | |
| | FPPKPKDTLMISRTPEVT | | | |
| | CVVVDVSHEDPEVKFNW | | | |
| | YVDGVEVHNAKTKPREE | | | |
| | QYNSTYRVVSVLTVLHQ | | | |
| | DWLNGKEYKCKVSNKAL | | | |
| | PLPEEKTISKAKGQPREP | | | |
| | QVYTLPPSREEMTKNQV | | | |
| | SLTCLVKGFYPSDIAVEW | | | |
| | ESNGQPENNYKTTPPVL | | | |
| | DSDGSFFLYSKLTVDKS | | | |
| | RWQQGNVFSCSVMHEA | | | |
| | LHNHYTQKSLSLSPG | | | |
| Humanized | QVQLQESGPGLVKPSQT | 84 | DIQMTQSPSSLSASVGDR | 38 |
| optimized | LSLTCTVTGYSITSDYTW | | VTITCKASQDVSSAVAWY | |
| antibody | NWIRQFPGKGLEWMGYI | | QQKPGKAPKLLIYSASYRY | |
| “AFF4-WT” | SYSGSYSYNPSLKSRVTI | | TGVPSRFSGSGSGTDFTF | |
| (human | SRDTSKNQFSLKLSSVTA | | TISSLQPEDFATYYCQQHY | |
| IgG | ADTAVYYCARSFSYSYG | | STPWTFGGGTKVEIKRTV | |
| format; | FAYWGQGTLVTVSSAST | | AAPSVFIFPPSDEQLKSGT | |
| wildtype | KGPSVFPLAPSSKSTSG | | ASVVCLLNNFYPREAKVQ | |
| human | GTAALGCLVKDYFPEPV | | WKVDNALQSGNSQESVT | |
| IgG) | TVSWNSGALTSGVHTFP | | EQDSKDSTYSLSSTLTLSK | |
| | AVLQSSGLYSLSSVVTVP | | ADYEKHKVYACEVTHQGL | |
| | SSSLGTQTYICNVNHKPS | | SSPVTKSFNRGEC | |
| | NTKVDKRVEPKSCDKTH | | | |
| | TCPPCPAPELLGGPSVFL | | | |
| | FPPKPKDTLMISRTPEVT | | | |
| | CVVVDVSHEDPEVKFNW | | | |
| | YVDGVEVHNAKTKPREE | | | |
| | QYNSTYRVVSVLTVLHQ | | | |
| | DWLNGKEYKCKVSNKAL | | | |
| | PAPIEKTISKAKGQPREP | | | |
| | QVYTLPPSREEMTKNQV | | | |
| | SLTCLVKGFYPSDIAVEW | | | |
| | ESNGQPENNYKTTPPVL | | | |
| | DSDGSFFLYSKLTVDKS | | | |
| | RWQQGNVFSCSVMHEA | | | |
| | LHNHYTQKSLSLSPG | | | |

⁶ The CDRs are shown underlined in Table 6. The CDRs in Table 6 are determined according to Kabat. The sequences of FR1, FR2 and FR3 of the respective variable domain heavy and light chain sequences are shown in italics.

In a yet further preferred embodiment, the antibody comprises:
(a) a heavy chain variable region comprising the amino acid sequence of SEQ ID NO: 30; and
(b) a light chain variable region comprising the amino acid sequence of SEQ ID NO: 20.

Preferably, the antibody comprises a heavy chain variable region sequence comprising the amino acid sequence of SEQ ID NO: 30 and comprises a light chain variable region sequence comprising the amino acid sequence of SEQ ID NO: 20.

Preferably, the antibody comprises a heavy chain variable region sequence which has the amino acid sequence of SEQ ID NO: 30 and comprises a light chain variable region sequence which has the amino acid sequence of SEQ ID NO: 20.

The antibody may further be selected from monoclonal antibodies, recombinantly produced antibodies, monospecific antibodies, multispecific antibodies, including bispecific antibodies, human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, single domain antibodies, monovalent antibodies, single chain antibodies or single-chain Fvs (scFv), camelid antibodies, affibodies, anticalins, affilins, atrimers, DARPins, FN3 scaffolds, such as adnectins and centyrins, fynomers, Kunitz domains, pronectins and OBodies, Fab fragments, F(ab′)2 fragments, disulfide-linked Fvs (dsFv), anti-idiotypic (anti-Id) antibodies, and antigen-binding fragments of any of the above, and/or wherein the antibody is comprised in a Chimeric Antigen Receptor (CAR).

The antibody may be selected from monoclonal antibodies. Monoclonal antibodies are monospecific antibodies that are identical because they are produced of one type of immune cell that are all clones of a single parent cell, e.g. produced by a single clone of B lymphocytes, or antibodies having the same amino acid sequence. “Monoclonal antibodies” and the production of monoclonal antibodies belong to the state of the art. In general, monoclonal antibodies can, for example, be prepared in accordance with the known method of Winter & Milstein [17]. An alternative to preparing monoclonal antibody-secreting hybridomas, a monoclonal antibody directed against a polypeptide of interest can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display library can be found in, for example, U.S. Pat. No. 5,223,409; WO 92/18619; WO 91/17271; WO 92/20791; WO 92/15679; WO 93/01288; WO 92/01047; WO 92/09690; WO 90/02809; [19]-[22].

The antibody may further be selected from synthetic antibodies. The term “synthetic antibody” describes any antibody entirely generated in vitro without any involvement of an animal. Methods for generating synthetic antibodies are well known to the person skilled in the art, such as recombinant protein production. However, while synthetic antibodies are generated in vitro, they may still be produced in vivo, such as using cell lines (such as mammal, insect or bacterial cell lines), in animals or hybridoma cells. Suitable methods are well known to the person skilled in the art.

The antibody may further be selected from recombinantly produced antibodies. The term “recombinantly produced antibodies” thereby comprises any antibody produced in vitro using DNA molecules generated by genetic recombination. Recombinant antibody production may be performed with cell lines (such as mammal, insect or bacterial cell lines), in animals or hybridoma cells. Methods for the recombinant production of antibodies are well known to the person skilled in the art. For example, antibody genes for immune-specific heavy and light antibody chains may be cloned into high-yield expression vectors, which subsequently are introduced into expression hosts (such as bacteria, yeast, insect or mammalian cells) to produce recombinant antibodies.

The antibody may further be selected from monovalent antibodies, monospecific antibodies and/or multispecific antibodies, including bispecific antibodies.

The valency of an antibody describes the number of antigen-binding sites present per molecule of antibody. The term “monovalent antibodies” therefore describes any antibody having one binding site to an antigen, epitope or cell type or tissue. A bivalent antibody has two binding sites to an antigen, epitope or cell type or tissue. A multivalent antibody has multiple, i.e. two or more, binding sites to an antigen, epitope or cell type or tissue, such as two, three, four or five.

The specificity of an antibody in general describes its ability to recognize a single antigen epitope and distinguish it from the rest. The term “monospecific antibody” describes any antibody having specificity to one antigen, epitope, cell type or tissue. For example, monoclonal antibodies are monospecific because they bind with each of their two antigen-binding arms to only one epitope. The term “bispecific antibody”, as used herein, may be understood in the broadest sense describing antibodies interacting with two different epitopes, such as an antibody comprising two functional antigen-binding domains having specificity to two different antigens, or, alternatively, two different epitopes on the same antigen. The bispecific antibody may be derived from two monoclonal antibodies. Optionally, these two different epitopes may be localized on the same antigen, but they may also be localized on two different antigens. Bispecific antibodies may be produced using conventional technologies, specific methods of which include production chemically, or from hybrid hybridomas and other technologies including, but not limited to, the technologies providing molecules, such as scFv, possessing antigen binding regions of different specificity with a peptide linker, such as a G4S linker, and knobs-into-holes engineering. The term “multispecific”, as used herein, may be understood in the broadest sense describing antibodies interacting with two or more different types of epitopes. Optionally, these epitopes may be localized on the same antigen or on two or more antigens. For example, on a multispecific antibody two or more, or three or more functional antigen-binding domains may be present and may have specificity for two or more, or three or more distinct antigens or distinct epitopes. Therefore, bispecific and multispecific antibodies target two and more antigens or epitopes, respectively.

The antibody may further be a human antibody, a humanized antibody and/or chimeric antibody. A chimeric antibody is an antibody in which at least one region of an immunoglobulin of one species is fused to another region of an immunoglobulin of another species by genetic engineering in order to reduce the antibody's immunogenicity. For example murine V_{L} and V_{H} regions may be fused to the remaining part of a human immunoglobulin. A particular type of chimeric antibodies are humanized antibodies. Humanized antibodies are produced by merging the DNA that encodes the CDRs of a non-human antibody with human antibody-producing DNA. The resulting DNA construct can then be used to express and produce antibodies that are usually not as immunogenic as the non-human parental antibody or as a chimeric antibody, since merely the CDRs are non-human. Further, the antibody may be a human antibody, i.e. the nucleic acid sequence of the antibody is entirely of human origin.

The use of a human, humanized or chimeric antibody is preferred for applications in vivo, in particular the human, e.g. for the prevention, treatment or diagnosis in vivo.

The antibody may further be selected from immunoglobulins. The term “immunoglobulin” describes any protein from the class of immunoglobulin that are produced by the immune system to neutralize substances foreign to the body. An immunoglobulin comprises at least one immunoglobulin (lg) domain.

The antibody may further be selected from tetrameric antibodies comprising two heavy chain and two light chain molecules, from an antibody light chain monomer, an antibody heavy chain monomer, an antibody light chain dimer, an antibody heavy chain dimer and/or an antibody light chain-antibody heavy chain pair.

The term “tetrameric antibodies comprising two heavy chain and two light chain molecules” describes any antibody complex comprising two heavy chain and two light chain molecules. These may comprise the complete heavy chain and/or light chain sequences of a full-length antibody or only parts of them. Tetrameric antibodies may especially refer to proteins that comprise two heavy (H) and two light (L) chains inter-connected by disulfide bonds which comprise: (1) in terms of the heavy chains, a variable region and a heavy chain constant region which comprises three domains, C_{H}1, C_{H}2 and C_{H}3; and (2) in terms of the light chains, a light chain variable region and a light chain constant region which comprises one domain, C_{L}. With regard to the term “tetrameric antibody”, any antibody is meant that has a typical overall domain structure of a naturally occurring antibody (i.e. comprising a heavy chain of three or four constant domains and a light chain of one constant domain as well as the respective variable domains). As described above, each domain may comprise further modifications, such as mutations, including substitutions, deletions, and/or insertions, which do not change the overall domain structure. For instance, mAb OV549.20 is a tetrameric antibody.

The term “antibody light chain monomer” describes any antibody comprising a light chain molecule only, but not comprising a heavy chain molecule. Accordingly, the term “antibody heavy chain monomer” describes any antibody comprising one heavy chain molecule only, but not comprising a light chain molecule. Accordingly, the term “antibody light chain dimer” describes a complex of two light chain monomers and the term “antibody heavy chain dimer” describes a complex of two heavy chain monomers. The term “antibody light chain-antibody heavy chain pair” describes any complex comprising a pair of a light chain monomer and a heavy chain monomer.

The antibody may further be selected from “single domain antibodies”. The term “single domain antibodies” describes an antibody fragment consisting of a single monomeric variable antibody domain, such as the variable domain of the light chain (V_{L}) or the variable domain of the heavy chain (V_{H}).

The antibody may further be selected from “single chain antibodies”. The term “single chain antibodies” describes an antibody fragment of a single polypeptide chain.

The antibody may further be selected from intrabodies and/or heteroconjugate antibodies. The term “intrabodies” describes any antibody targeting intracellular proteins within a cell. Methods for transferring an intrabody in the target cell to allow the binding of intracellular target proteins are well known to the person skilled in the art, such as the direct expression of the intrabody by the target cell as applied in gene therapy. “Heteroconjugate antibodies” are complexes of two or more antibodies (e.g. monoclonal antibodies, Fab or scFv) of different specificities that are covalently linked.

The antibody may further be selected from camelid antibodies, affibodies, anticalins, affilins, atrimers, DARPins, FN3 scaffolds, such as adnectins and centyrins, fynomers, Kunitz domains, pronectins and OBodies, and/or anti-idiotypic (anti-Id) antibodies. The term “camelid antibody” describes any antibody having the structure of an antibody derived from the Camelidae family of mammals (such the llamas, camels, and alpacas), such as an antibody lacking any light chain and consisting of two identical heavy chains. The term “affibodies” describes any antibody mimetic protein, which is able to bind a large number of antigens with high affinity. For example, affibodies may be based on immunoglobulin binding domains of proteins, such as the Z domain of protein A from Staphylococcus aureus. Further examples are well known to the person skilled in the art. Anticalins, affilins, atrimers, DARPins, FN3 scaffolds, such as adnectins and centyrins, fynomers, Kunitz domains, pronectins and OBodies are further scaffolds which are known in the art and which can be used according to the invention. The scaffolds are for example described in [16]. The term “anti-idiotypic (anti-Id) antibodies” describes any antibody, which is capable of binding to the idiotype of another antibody.

An “antigen-binding fragment” of an antibody is a fragment of an antibody, which preferably exhibits essentially the same function and specificity as the complete antibody of which the fragment is derived from. Limited proteolytic digestion with papain usually cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their interchain disulfide bond, is the crystallizable fragment (Fc). The Fc contains carbohydrates, complement-binding, and FcR-binding sites. Limited pepsin digestion yields a single F(ab′)₂ fragment containing both Fab pieces and the hinge region, including the H—H interchain disulfide bond. F(ab′)₂ is divalent for antigen binding. The disulfide bond of F(ab′)₂ may be cleaved in order to obtain Fab′. Moreover, the variable regions of the heavy and light chains can be fused together to form a single chain variable fragment (scFv).

As the first generation of full sized antibodies may present some problems, many of the second generation antibodies may comprise only fragments of the antibody. Variable domains (Fvs) are the smallest fragments with an intact antigen-binding domain consisting of one V_{L} and one V_{H}. Such fragments, with only the binding domains, can be generated by enzymatic approaches or expression of the relevant gene fragments, e.g. in bacterial and eukaryotic cells. Different approaches can be used, e.g. either the Fv fragment alone or Fab-fragments comprising one of the upper arms of the “Y” that includes the Fv plus the first constant domains. When only the variable fragments are used, these are usually stabilized by introducing a polypeptide link between the two chains which results in the production of a single chain Fv (scFv). Alternatively, disulfide-linked Fv (dsFv) fragments may be used. The binding domains of fragments can be combined with any constant domain in order to produce full length antibodies or can be fused with other proteins and polypeptides.

A preferred recombinant antibody fragment is the single-chain Fv (scFv) fragment. In general, it has a high affinity for its antigen and can be expressed in a variety of hosts. These and other properties make scFv fragments not only applicable in medicine, but also of potential for biotechnological applications. As detailed above, in the scFv fragment the VH and VL domains are joined with a hydrophilic and flexible peptide linker, which improves expression and folding efficiency. Usually linkers of about 15 amino acids are used, of which the (Gly₄Ser)₃ linker has been used most frequently. scFv molecules might be easily proteolytically degraded, depending on the linker used. With the development of genetic engineering techniques these limitations could be practically overcome by research focused on improvement of function and stability. An example is the generation of disulfide-stabilized (or disulfide-linked) Fv fragments where the V_{H}-V_{L} dimer is stabilized by an interchain disulfide bond. Cysteines are introduced at the interface between the V_{L} and V_{H} domains, forming a disulfide bridge, which holds the two domains together.

scFvs can be complexed into dimers (diabodies), trimers (triabodies) or larger aggregates such as TandAbs and Flexibodies.

Antibodies with two binding domains can for example be created either through the binding of two scFv with a simple polypeptide link (scFv) 2 or through the dimerization of two monomers (diabodies). The simplest designs are diabodies that have two functional antigen-binding domains.

Also, antibody formats comprising four variable domains of heavy chains and four variable domains of light chains have been developed. Examples of these include TandAbs and Flexibodies (Affimed Therapeutics AG, Heidelberg. Germany). Due to its four binding domains the TandAb usually shows better binding properties compared to antibody formats comprising only two binding domains, such as e.g. diabodies. Flexibodies are a combination of scFv with a diabody multimer motif resulting in a multivalent molecule with a high degree of flexibility for joining two molecules which are quite distant from each other on the cell surface.

The antibody may also be selected from antigen-binding fragments of any of the above mentioned molecules. As specified above, an “antigen-binding fragment” of an antibody is a fragment of an antibody, which exhibits essentially the same antigen binding activity and specificity as the complete antibody of which the fragment is derived from. The antigen-binding fragment is usually understood as polypeptide which comprises at least one antigen-binding fragment of a full-length antibody. In general, antigen binding fragments consist of at least the variable domain of the heavy chain and the variable domain of the light chain, arranged in a manner that both domains together are able to bind to the specific antigen.

Further, certain binding molecules or antigen-binding fragments of monoclonal antibodies including, but not limited to, Fv, scFv, diabody molecules or domain antibodies (Domantis) may be stabilized by incorporating disulfide bridges to line the VH and VL domains.

The antibody may further be comprised in a complex with further immunoglobulin molecules or fragments thereof (such as further antibodies) or non-immunoglobulin molecules. For example, the antibody may form a homomultimer with further identical antibodies. Preferably, antibody is comprised in a Chimeric Antigen Receptor (CAR). The term “CAR” describes any receptor protein, usually on a T cell, which has been specifically designed to allow T cells to target a specific antigen. Methods for preparing CARs are well known to the person skilled in the art.

In a yet further preferred embodiment, the antibody is selected from monoclonal antibodies, recombinantly produced antibodies, monospecific antibodies, multispecific antibodies, including bispecific antibodies, human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, single domain antibodies, monovalent antibodies, single chain antibodies or single-chain Fvs (scFv), camelid antibodies, affibodies, anticalins, affilins, atrimers, DARPins, FN3 scaffolds, such as adnectins and centyrins, fynomers, Kunitz domains, pronectins and OBodies, Fab fragments, F(ab′) 2 fragments, disulfide-linked Fvs (dsFv), anti-idiotypic (anti-Id) antibodies, and antigen-binding fragments of any of the above, and/or wherein the antibody is comprised in a Chimeric Antigen Receptor (CAR).

In another yet further preferred embodiment, the antibody is selected from monoclonal antibodies, recombinantly produced antibodies, monospecific antibodies, multispecific antibodies, including bispecific antibodies, human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, single domain antibodies, monovalent antibodies, single chain antibodies or single-chain Fvs (scFv), camelid antibodies, Fab fragments, F(ab′)2 fragments, disulfide-linked Fvs (dsFv), anti-idiotypic (anti-Id) antibodies, and antigen-binding fragments of any of the above, and/or wherein the antibody is comprised in a Chimeric Antigen Receptor (CAR).

Moreover, the antibody may comprise heavy and/or light chain constant regions. The heavy chain constant region may be selected from the group of human immunoglobulins selected from IgA, IgD, IgE, IgG or IgM, including any subclass of these isotypes. Preferably, the heavy chain constant region is selected from the group of human immunoglobulins consisting of IgG and IgA, more preferably the heavy chain constant region is selected from the group of human immunoglobulins consisting of IgG1, IgG2, IgG3, IgG4, IgA1, and IgA2. Since the antibody may be expressed and produced recombinantly, the antibody may also comprise two different constant regions of heavy chains, e.g. one IgG1 and one IgG2 heavy chain, or heavy chains from different species. However, the heavy chains preferably are from the same species. Furthermore, the antibody may comprise either an IgGκ or an IgGλ light chain constant region. Preferably, the light chain constant region is selected from the group of human immunoglobulins consisting of IgGκ and IgGλ.

In a yet further preferred embodiment, the antibody comprises heavy and/or light chain constant regions, preferably wherein the heavy chain constant region is selected from the group of human immunoglobulins consisting of IgG1, IgG2, IgG3, IgG4, IgA1, and IgA2, and/or, wherein the light chain constant region is selected from the group of human immunoglobulins consisting of IgGκ and IgGλ.

In a yet further preferred embodiment, the antibody comprises a heavy chain constant region, wherein the heavy chain constant region is selected from the group of human immunoglobulins consisting of IgG1, IgG2, IgG3 and IgG4.

In a preferred embodiment, the antibody comprises a heavy chain constant region, wherein the heavy chain constant region is a human IgG1 constant region.

The heavy chain constant region may further be a variant of a wild type human IgG heavy chain constant region, preferably wherein the variant human IgG heavy chain constant region binds to one or more of human Fc gamma receptors. In general Fc receptors are surface proteins of certain cells contributing to the immune system.

There are several classes of Fc receptors, which among others may be distinguished by the antibody type they interact with. The term “Fc gamma receptor” therefore describes Fc receptors binding antibodies having an IgG constant region. The class of Fc gamma receptors further comprises several subclasses, such as FcγRI, FcγRIIA, FcγRIIB, FcγRIIIA or FcγRIIIB, which generally differ in their structure and affinity to IgG and the different IgG subclasses. Preferably, the variant human IgG heavy chain constant region of the antibody of the invention binds to one or more of human Fc gamma receptors selected from the group consisting of FcγRI, FcγRIIA, FcγRIIIA. Also preferably, the variant human IgG heavy chain constant region binds to one or more of human Fc gamma receptors selected from the group consisting of FcγRI, FcγRIIA, FcγRIIIA with higher affinity than the wild type human IgG heavy chain constant region binds to the human Fc gamma receptors. “Binding with higher affinity” means that the binding of the antibody according to the invention to one or more of the human Fc gamma receptors selected from the group consisting of FcγRI, FcγRIIA, FcγRIIIA is at least 2-fold, preferably at least 3-fold stronger than the binding of the wild type human IgG heavy chain constant region to the human Fc gamma receptors, as determined e.g. by methods known to the person skilled in the art, such as Western Blot analysis, ELISA, or surface plasmon resonance.

In a yet further preferred embodiment, the heavy chain constant region is a variant of a wild type human IgG heavy chain constant region, preferably wherein the variant human IgG heavy chain constant region binds to one or more of human Fc gamma receptors selected from the group consisting of FcγRI, FcγRIIA, FcγRIIIA with higher affinity than the wild type human IgG heavy chain constant region binds to the human Fc gamma receptors.

In another aspect, the present invention relates to an antibody that specifically binds to the same epitope of human L1CAM as the antibody of the invention and/or competes for binding of the antibody of the invention to human L1CAM, preferably wherein the epitope is within the fibronectin type III domains 1-3 (FN III 1-3) of human L1CAM.

All features specified above for the antibody of the of the invention are considered to also relate to the antibody of the further aspects of the invention.

The antibody may specifically bind to the same epitope of human L1CAM as the antibody of the invention.

The term “epitope” describes the part of an antigen that is recognized by antibodies or related binding molecules. For example, the epitope is the specific piece of the antigen that an antibody binds to. Epitopes may be conformational epitopes or linear epitopes. A conformational epitope is usually composed of discontinuous sections of the antigen's amino acid sequence. These epitopes interact with the paratope (binding site on antibody) based on the 3-D surface features and shape or tertiary structure of the antigen. The proportion of epitopes that are conformational is unknown. Linear epitopes are epitopes that are usually recognized by an antibody via their amino acid sequence or primary structure.

It was shown that L1-OV549.20 binds to and recognizes an epitope within the fibronectin type III domains 1-3 (FN III 1-3) of L1CAM [6]. Methods for determining an epitope bound and recognized by a binding molecule are described in the prior art. A recognized epitope may be determined by constructing a series of L1CAM-Fc proteins carrying distinct Ig domains. For fine mapping, recombinant V5-tagged L1CAM fragments can be used, as for example as described in [23]. The recombinant proteins can be used in ELISA or in Western blot analysis for epitope mapping. In general, methods for determining the epitope of a given antibody are known in the art and include the preparation of synthetic linear peptides of a given region of interest and the subsequent testing whether the antibody binds to said peptides (see [24]. Alternatively, different recombinant proteins covering the region of interest can be produced and tested for the binding of the antibody [25].

The antibody may alternatively or additionally compete for binding of the antibody of the invention to human L1CAM, preferably wherein the epitope is within the fibronectin type III domains 1-3 (FN III 1-3) of human L1CAM.

Competition for binding of the antibodies may generally be determined by assays known to a skilled person, such as Competitive binding assays. Competitive binding assays are usually based on antibody-antigen interactions in which the number of antigen binding sites on the antibody is limited in comparison to the amount of the different competing antibodies. For example, Competitive binding assays may have the form of immunoassays.

Human L1CAM protein usually comprises an N-terminal extracellular portion of six immunoglobulin domains (Ig I to Ig VI), followed by five fibronectin type III domains (FN III 1-5), a transmembrane helix and a small C-terminal intracellular domain. Preferably the epitope is within the fibronectin type III domains 1-3 (FN III 1-3) of human L1CAM.

Preferably, the antibody of any of the aspects of the invention
(i) binds to human L1CAM within the fibronectin type III domains 1-3 (FN III 1-3) of L1CAM.

Preferably, the antibody of any of the aspects of the invention
(ii) binds to human L1CAM with an affinity (KD) of 20 nM or less, 10 nM or less, or 1 nM or less.

Preferably, the antibody of any of the aspects of the invention
(iii) binds to cynomolgus monkey L1CAM with an affinity (KD) of 20 nM or less, 10 nM or less, or 1 nM or less.

Methods for determining the binding affinity of an antibody are well known to the person skilled in the art and exemplarily also described above in the context of specific binding of an antibody. For example, surface plasmon resonance using a Biacore® device may be used. For example, the affinity (KD) is determined at room temperature. For example, binding affinity may be determined as described in Example 2 below.

Preferably, the antibody of any of the aspects of the invention
(iv) inhibits the migration of tumor cells on a fibronectin-coated surface in vitro.

Methods for determining the migration of tumor cells on a fibronectin-coated surface in vitro are well known to the person skilled in the art. For example, fluorescently labeled tumor cells may be seeded on the edge of a fibronectin-coated well. After 48 hours, the migration of tumor cells towards the center of the well may be determined. Preferably, when the antibody of any of the aspects of the invention is applied to tumor cells, the migration of these tumor cells on a fibronectin-coated surface in vitro is inhibited.

In this context, inhibition of migration means that when the antibody of any of the aspects is applied to tumor cells, the migration of these tumor cells on a fibronectin-coated surface in vitro after 48 hours is reduced by at least 10%, at least 20%, at least 30%, at least 40%, at least 50%, at least 60%, at least 70%, at least 80%, at least 90%, at least 95% or by at least 99% in comparison to the migration of tumor cells of the same cell type treated with an isotype control antibody on a fibronectin-coated surface in vitro.

Further, the half maximal inhibitory concentration (IC₅₀) for the antibody of the present invention for inhibiting cancer cell migration of HCT116 tumor cells may be from 1×10⁻⁴ M to 1×10⁻⁸ M and preferably from 1×10⁻⁴ M to 1×10⁻⁷ M. Optionally, the half maximal inhibitory concentration (IC₅₀) for the antibody of the present invention for inhibiting cancer cell migration of HCT116 tumor cells may also be from 2×10⁻⁵ M to 1×10⁻⁶ M, preferably from 2×10⁻⁵ M to 4×10⁻⁶ M Preferably, the tumor cells are HCT116 tumor cells.

Preferably, the antibody of any of the aspects of the invention
(v) inhibits proliferation of SKOV-3, Panc-1, and/or HCT-116 tumor cells in vitro.

Methods for determining the proliferation of SKOV-3, Panc-1, and/or HCT-116 tumor cells in vitro are well known to the person skilled in the art. For example, such proliferation assay may be applied to determine the number of cells over time, the number of cellular divisions, the metabolic activity, or DNA synthesis. Preferably, tumor cell proliferation is monitored by detecting cell confluency via microscopic devices over time, such as determining (e.g. by counting) the number of cells over time, e.g. after 24 h, 48 h, 72 h or 96 h.

In this context, inhibition of proliferation in vitro means that when the antibody of any of the aspects of the invention is applied to tumor cells, the proliferation of these tumor cells after 24 h, 48 h, 72 h or 96 hours is reduced by at least 10%, at least 20%, at least 30%, at least 35%, at least 40%, at least 45%, at least 50%, at least 55%, at least 60%, at least 65%, at least 70%, at least 80%, at least 90%, at least 95% or by at least 99% in comparison the proliferation in vitro of tumor cells of the same cell type treated with an isotype control antibody.

Preferably, the antibody of any of the aspects of the invention
(vi) inhibits primary tumor growth in a mouse SKOV-3ip xenograft model.

Methods for determining primary tumor growth in a mouse SKOV-3ip xenograft model are well known to the person skilled in the art. For example in a SKOV-3ip xenograft model, 5×10⁶ SKOV3 tumor cells (i.p.) may be injected into NMRI-nu/nu mice. After 5 days mice may be randomized into different groups and treatments may be started. Anti-L1CAM antibody or control antibody may be injected intraperitoneally three times per week at doses of 10 mg/kg over a period of 6 weeks. Upon sacrifice of mice the treatment efficacy may be assessed comparing tumor weights and ascites volumes between treated groups and the control groups

In this context, inhibition of primary tumor growth in a mouse model is understood as that when the antibody of any of the aspects of the invention is applied to the tumor mouse model, the tumor growth at the time point of meeting the endpoint requirements is reduced by at least 10%, at least 20%, at least 30%, at least 40%, at least 50%, at least 55%, at least 60%, at least 70%, at least 80%, at least 90%, at least 95% or by at least 99% in comparison to the tumor growth in the same model treated with an isotype control antibody or a vehicle.

Preferably, the antibody of any of the aspects of the invention
(vii) reduces metastasis formation in a mouse MDA-MB-231 xenograft model.

MDA-MB-231 is a highly invasive model cell line derived from a metastatic site of breast cancer. Methods for determining metastasis formation in the MDA-MB-231 xenograft model are well-known to the person skilled in the art. For example, MDA-MB-231-luc2 luciferase expressing breast cancer cells may be injected into the tail vein of a mouse (e.g. about 5×10⁵ cells per mouse). The mice may further be treated with an antibody according to the invention (such as OV549.20) or a vehicle (control), e.g. with a dose of 10 mg/kg three times per week, starting 3 days before MDA-MB-231-luc2 injection. In vivo imaging of luciferase activity may be conducted every 7 days and compared in control and antibody-treated cells to monitor the formation of metastases in lungs and other visceral organs.

In this context, reduction of metastasis formation in the mouse MDA-MB-231 xenograft model is understood as that when the antibody of any of the aspects of the invention is applied to MDA-MB-231-luc2 injected mice the metastasis formation after 20 to 30 days of exposure to MDA-MB231-luc2 cells is reduced by at least 10%, at least 20%, at least 30%, at least 40%, at least 50%, at least 55%, at least 60%, at least 70%, at least 80%, at least 90%, at least 94%, at least 95% or by at least 99% in comparison the metastasis formation in the same model treated with a control vehicle.

Preferably, the antibody of any of the aspects of the invention
(viii) exhibits ADCC activity in vitro and/or binds to FcγRIIIa receptors in vitro.

The term “Antibody dependent cellular cytotoxicity (ADCC)” describes the mechanism by which a target cell, which has been bound and thereby marked by a specific antibody, is consequently lysed by effector cells (such as natural killer (NK) cells) of the immune system. Preferably, the antibody of the invention exhibits ADCC activity in vitro, i.e. it is capable of mediating the ADCC immune response when bound to a target cell in in vitro assays. Such in vitro assays are well known to the person skilled in the art. For example, suitable assays employ effector cells, such as NK cells, capable of inducing lysis of target cells bound by antibodies that are applied in different effector to target (E:T) cell ratios. In such case, final analysis may for example be performed using a Real-Time Cell Analysis device.

Alternatively or in combination to exhibiting ADCC activity, the antibody of any of the aspects of the invention preferably binds to FcγRIIIa receptor in vitro. Suitable in vitro binding assays for antibodies and receptors that may be used for determining the binding between the antibody of the invention and FcγRIIIa receptors in vitro are well known to the person skilled in the art and e.g. described in the context of specific binding above. For example, Western Blot analysis, ELISA, or surface plasmon resonance may be used.

Preferably, the antibody of any of the aspects of the invention
(ix) exhibits binding to FcRn in vitro.

The neonatal Fc receptor (FcRn) is a Fc receptor protein, which is capable of binding e.g. IgG, and is usually expressed by e.g. endothelial cells where it supports recycling of serum IgG and albumin. Suitable in vitro binding assays for antibodies and receptors that may be used for determining the binding between the antibody of the invention and FcRn in vitro are well known to the person skilled in the art or e.g. described in the context of specific binding above.

Preferably, the antibody of any of the aspects of the invention
(x) does not cross-react with human CHL1, human NrCAM, and/or human neurofascin in vitro.

L1CAM belongs to the L1-family of proteins, which in total comprises four different L1-like proteins, which are all cell adhesion molecules (CAMs) and members of the immunoglobulin superfamily. Besides L1CAM, the three additional members of the L1-family are close homologue to L1CAM (CHL1), Neuronal cell adhesion molecule (NrCAM) and neurofascin.

An antibody of any of the aspects which “does not cross-react with” human CHL1, human NrCAM, and/or human neurofascin in vitro is understood as an antibody which binds to human L1CAM, but does not substantially bind to any one of human CHL1, human NrCAM, and/or human neurofascin in vitro. In one embodiment, an antibody of any of the aspects of the invention which does not cross-react with human CHL1, human NrCAM, and/or human neurofascin in vitro shows only very low or unspecific binding to human CHL1, human NrCAM, and/or human neurofascin in vitro with a binding affinity of a K_{D}-value of 1×10⁻⁷ M or higher or 1×10⁻⁶ M or higher (until no more binding affinity is detectable). In one embodiment, the binding affinity is determined with a standard binding assay, such as an ELISA assay, as in the examples, or a surface plasmon resonance technique. For example, the binding affinity is determined at room temperature. Further suitable methods for determining the binding of the antibody of any of the aspects of the invention and CHL1, NrCAM and/or neurofascin are well known to the person skilled in the art. For example, methods such as Western Blot analysis, or determining shifts in the fluorescent signal in cytometer-based assays may be used.

In a yet further preferred embodiment, the antibody of any of the aspects of the invention described herein:
(i) binds to human L1CAM within the fibronectin type III domains 1-3 (FN III 1-3) of L1CAM, and/or
(ii) binds to human L1CAM with an affinity (K_{D}) of 20 nM or less, 10 nM or less, or 1 nM or less, and/or
(iii) binds to cynomolgus monkey L1CAM with an affinity (K_{D}) of 20 nM or less, 10 nM or less, or 1 nM or less, and/or
(iv) inhibits the migration of tumor cells on a fibronectin-coated surface in vitro, and/or
(v) inhibits proliferation of SKOV-3, Panc-1, and/or HCT-116 tumor cells in vitro; and/or
(vi) inhibits primary tumor growth in a mouse SKOV-3ip xenograft model, and/or
(vii) reduces metastasis formation in mouse MDA-MB-231 xenograft model, and/or
(viii) exhibits ADCC activity in vitro and/or binds to FcγRIIIa receptors in vitro, and/or
(ix) exhibits binding to FcRn in vitro, and/or
(x) does not cross-react with human CHL1, human NrCAM, and/or human neurofascin in vitro.

Further, the antibody according to any of the aspects of the invention may be humanized. Preferably, the antibody according to the invention is partially or completely humanized. Methods for humanizing an antibody are well-known to the person skilled in the art, for example by introducing the sequences of the CDRs of the antibody of the invention into the sequence of a human antibody. Optionally, in addition, one or more non-human positions, such as 1, 2, 3, 4, 5, or 6, such as up to 10, non-human positions, may be re-introduced into the human framework sequences.

Preferably, the antibody according to any of the aspects of the invention is humanized.

In another preferred embodiment of any of the aspects of the invention, the antibody is a multispecific or bispecific antibody and/or is a humanized antibody.

Methods for the production of antibodies, such as the antibody of the invention, are well known to the person skilled in the art. For example, antibodies may be produced by making hybridoma cells. Methods for the production of hybridoma cells as well as methods for the production of antibodies with the help of hybridoma cells are well-known to the person skilled in the art. Generally, mice are injected with the desired antigen and killed after a few days or weeks in order to isolate the spleen cells secreting the antibody against the desired antigen. In general, fusion of these antibody-secreting spleen cells with immortal non-secreting myeloma cells results in hybridoma cells. These hybridoma cells are then usually screened and the hybridoma producing the desired antibody is selected. The selected hybridoma may then be cultured in vivo or in vitro and the desired antibody can be isolated. Preferably, the antibodies herein are produced recombinantly in suitable host cells. The DNA encoding the antibody of interest can be placed into expression vectors, which are then transfected into host cells such as E. coli cells, simian COS cells, Chinese hamster ovary (CHO) cells (e.g., CHO cells from the CHO GS System™ (Lonza)), or myeloma cells that do not otherwise produce immunoglobulin protein, to obtain the synthesis of the antibody.

### Antibody Conjugates/Antibody-Drug Conjugates (ADCs)

The antibody of any of the aspects of the invention may further be linked to one or more chemical moiety(ies) in order to form an antibody conjugate. For example, the antibody may be linked to such one or more chemical moiety(ies) by any chemical bonding known to the person skilled in the art, such as ionic bonding and/or covalent bond and/or by any suitable intermolecular bonding, such as hydrogen bond and/or van der Waals forces.

Further, the chemical moiety(ies) to which the antibody of any of the aspects of the invention may be linked to may be any chemical moiety(ies) or substance(s) suitable for application in an antibody-conjugate known to the person skilled in the art. For example, the antibody may be linked to a therapeutically active substance, preferably to a chemotherapeutic compound, a cytotoxic compound, a cytostatic compound, a cytokine, a nanoparticle, a radioisotope, and/or an oncolytic virus.

The term “therapeutically active substance” describes any biologically active substance, i.e. a substance causing an effect in a living matter. When used in a pharmaceutical drug, the therapeutically active substance is e.g. responsible for the activity of the medicine. Methods for determining the effect of a substance on living matter are well known to the person skilled in the art.

The term “chemotherapeutic compound” describes any substance that may be used in cancer treatment as part of a standardized chemotherapy regimen as known to the person skilled in the art.

The term “cytotoxic compound” describes any substance being toxic to cells, e.g. by causing apoptosis or necrosis. Suitable examples of cells are known by the person skilled in the art, such as immune cells. The cytotoxicity of a compound may be measured by common cytotoxicity assays known to the person skilled in the art. Examples of cytotoxic agents include, for example, small molecule toxins or enzymatically active toxins of bacteria (such as Diptheria toxin, Pseudomonas endotoxin and exotoxin, Staphylococcal enterotoxin A), fungi (e.g., α-sarcin, restrictocin), or plants (e.g., abrin, ricin, modeccin, viscumin, pokeweed anti-viral protein, saporin, gelonin, momoridin, trichosanthin, barley toxin, Aleurites fordii proteins, dianthin proteins, Phytolacca mericana proteins (PAPI, PAPII, and PAP-S), Momordica charantia inhibitor, curcin, crotin, Saponaria officinalis inhibitor, gelonin, mitogellin, restrictocin, phenomycin, neomycin, and the tricothecenes), a mitotic inhibitor or a DNA damaging agent.

The term “cytostatic compound” describes any substance being capable of inhibiting cell growth. The capability of inhibiting cell growth by a compound may be measured by common cell growth assays known to the person skilled in the art.

Suitable chemotherapeutic compounds include alkylating agents, alkyl sulfonates, aziridines, ethylenimines and methylamelamines, acelogenins, a camptothecin, bryostatin, ostatin, callystatin, CC-1065, cryptophycins, dolastatin, duocarmycin, eleutherobin, pancratistatin, a sarcodictyln, spongistatin, nitrogen mustards, antibiotics, enediyne antibiotics, dynemicin, bisphosphonates, esperamicin, chromoprotein enediyne antibiotic chromophores, aciacinomysins, actinomycin, authramycin, azaserine, bleomycins, cactinomycin, carahtein, carminomycin, carzinophilin, chromomycinis, dactinomycin, daunorubicin, detorubicin, 6-diazo-5-oxo-L-norleucine, ADRIAMYCIN® doxorubicin, epirubicin, esorubicin, idarubicin, marcellomycin, mitomycins, niycophenolie acid, nogalamycin, olivomycins, peplomycin, potfiromycin, puromycin, quelamyein, rodorubicin, streptonigrin, streptozoein, tubercidin, ubenimex, zinostatin, zorubicin; anti-metabolites, erlotinib, vemurafenib, erizotinib, sorafenib, ibrutinib, enzaiutamide, folic acid analogues, purine analogs, androgens, anti-adrenals, folic acid replentsher such as frolinic acid, acegiatone, aldophosphamide glycoside, aminolevulinic acid, emluracil, anisacrine, bestrabucil, bisantrene, edatraxate, defofamine, demecolcine, diaziquone, elfornithine, elliptinium acetate, an epothilone, etoglucid, gallium nitrate, hydroxyurea, lentinan, lonidainine, maytansinoids, mitoguazone, mitoxantrone, mopidannioi, nitraerine, pentostatin, phenamet, pirarobicin, iosoxantrone, podophyllinic acid, 2-ethylhydrazide, procarbazine, PSK® polysaccharide complex, razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannoniustine; miiobronitol; mitolactol; pipobroman; gacytosine; arabinoside (“Ara-C”); cyclophosphamide; thiotepa; taxoids, chlorambucil; gemcitabine; 6-thioguanine; mercaptopurine; methotrexate; platinum analogs, vinblastine; platinum; etoposide; ifosfamide; mitoxantrone; vincristine; vinorelbine; novantrone; teniposide; edatrexate; daunomycin; aminopterin; xeloda; ibandronate; irinotecan, topoisomerase inhibitor RFS 2000; difluorometlhylornithine; retinoids; capecitabine; combretastatin; leucovorin; oxaliplatin; and pharmaceutically acceptable salts, acids or derivatives of any of the above.

The term “cytokine” describes a substance having the capability of influencing cell growth and/or cell differentiation. Suitable cytokines are well known to the person skilled in the art, such as interferons, interleukins, colony-stimulating factors, tumor necrosis factors or chemokines. Methods for measuring cell growth or cell differentiation are well known to the person skilled in the art. Suitable cytokines include for example IL-2, G-CSF, GM-CSF and TNF-α.

The term “nanoparticle” describes any particle having a diameter of 1 to 100 nm. Methods for determining the diameter of a particle are also well known to the person skilled in the art.

The term “radioisotope” describes any substance being an instable or metastable isotope of a natural or an artificial element. Suitable radioisotopes are preferably also applicable for treatment in the human or animal body. Suitable radioisotopes include ³H, ¹⁴C, ³²P, ³⁵S, ³⁶Cl, ⁵¹Cr, ⁵⁷Co, ⁵⁸Co, ⁵⁹Fe, ⁶⁷Cu, ⁹⁰Y, ⁹⁹Tc, ¹¹¹ In, ¹¹⁷Lu, ¹²¹I, ¹²⁴I, ¹²⁵I, ¹³¹I, ¹⁹⁸Au, ²¹¹At, ²¹³Bi, ²²⁵Ac and ¹⁸⁶Re.

The term “oncolytic virus” describes any virus preferably targeting cancer cells, e.g. leading to the infection and killing of these cancer cells, usually by oncolysis. Suitable oncolytic viruses are e.g. genetically adapted Herpes simplex virus, adenovirus, vaccinia virus, vesicular stomatitis virus, poliovirus, reovirus, senecavirus, echovirus, Semliki Forest virus, maraba virus and/or Coxsackievirus.

Alternatively, or in combination therewith, the antibody may be linked to a diagnostic compound, preferably selected from a radioisotope, a chemoluminescent compound, a fluorescent compound, a dye or an enzyme.

The term “diagnostic compound” describes any substance suitable for being applicable in diagnostic methods for the human or animal body. For example, diagnostic compounds may be used to stain tissues, cells or other materials in various analytical methods, such as fluorescent imaging or spectroscopy. For example, diagnostic compounds may be used to specifically stain cancer cells. Further diagnostic applications are well known to the person skilled in the art.

The term “chemoluminescent compound” describes any substance being capable of emitting light as a result of a chemical reaction. Methods and devices for detecting chemoluminescence are well known to the person skilled in the art.

The term “fluorescent compound” describes any substance re-emitting light in response to light excitation. Usually, such substances are also called fluorophores. Suitable fluorescent compounds may e.g. be substrates of enzymes or probes. Methods and devices for detecting fluorescence are well known to the person skilled in the art. Fluorescence compounds include, for example, reactive and conjugated probes e.g. Aminocoumarin, Fluorescein and Texas red, Alexa Fluor dyes, Cy dyes and DyLight dyes.

The term “dye” describes any substance, which is colored due to absorbing not all wavelengths of visible light and which is capable of chemically binding to a target molecule. It may thereby be used to mark and visualize a usually colorless target molecule.

The term “enzyme” describes any protein that is capable of catalyzing a chemical reaction. Suitable enzymes are known in the art and include horseradish peroxidase (HRP).

In another preferred embodiment of any of the aspects of the invention, the antibody is:
(a) linked to a therapeutically active substance,
   preferably to
   a chemotherapeutic compound,
   a cytotoxic compound,
   a cytostatic compound,
   a cytokine,
   a nanoparticle,
   a radioisotope, and/or
   an oncolytic virus,
   and/or
(b) linked to a diagnostic compound, preferably selected from a radioisotope, a chemoluminescent compound, a fluorescent compound, a dye or an enzyme.

It is preferred that the therapeutically active substance of (a) and/or the diagnostic compound of (b) is selected from a radioisotope, a chemotherapeutic compound, a cytotoxic compound, and/or a cytostatic compound.

Moreover, the antibody of the invention may be covalently linked to the therapeutically active substance of (a) or a chelator thereof or the diagnostic compound of (b) or a chelator thereof.

The term “covalently linked” describes any chemical bonding comprising the sharing of electron pairs between atoms. Examples of such covalent links are cleavable bonds (such as disulfide bonds, hydrazone-based bonds or peptide bonds) or non-cleavable bonds (such as thioether bonds). Preferably, the covalent bond is stable when applied to the body and circulating therein and only becomes cleavable within the target cell or when arriving at the target tissue. The linker may be a cleavable linker or may be a non-cleavable linker.

The term “chelator” describes any substance comprising two or more free pairs of electrons and, therefore, being capable of forming two or more coordinate bonds with metal ions. Chelators may be organic compounds (such as ethylenediaminetetraacetic acid (EDTA), 1,4,8,11-tetraazacyclotetradecane-1,4,8,11-tetraacetic acid (TETA) or 1,4,7,10-tetraazacyclododecane-1,4,7,10-tetraacetic acid (DOTA)). For example, the antibody according to any of the aspects may be linked to a chelator which is further linked to a radioisotope, such as positron- and gamma-emitting radiometals enabling sensitive and quantitative molecular Positron Emission Tomography (PET) and Single Photon Emission Computed Tomography (SPECT) imaging of antibody distribution in vivo. In another example, the antibody according to any of the aspects may be linked to a chelator which is further linked to a radioisotope allowing targeted radioisotope delivery directly at the position of interest in the body (e.g. at the tumor location).

Further, the antibody of the invention may be covalently linked to the therapeutically active substance of (a) or a chelator thereof or the diagnostic compound of (b) or a chelator thereof via a linker. Such antibodies of the invention which are linked to a therapeutically active substance or a chelator thereof via a linker are herein also referred to as “Antibody-Drug conjugate”, “antibody conjugate”, “antibody drug conjugate” or “ADC”.

In the context of an “Antibody-Drug conjugate”, “antibody conjugate”, “antibody drug conjugate” or “ADC” herein, it is also referred to the antibody moiety as “antibody portion”.

The term “linker” describes any molecule suitable for connecting the antibody of the invention to the therapeutically active substance of (a) or a chelator thereof or the diagnostic compound of (b) or a chelator thereof, wherein the linker preferably is connected to the antibody of the invention and/or to the therapeutically active substance of (a) or a chelator thereof or the diagnostic compound of (b) or a chelator thereof via a covalent bond.

Preferably, the linker provides a stable connection between the antibody of the invention to the therapeutically active substance of (a) or a chelator thereof or the diagnostic compound of (b) or a chelator thereof, when applied to the body and circulating therein and only becomes cleavable within the target cell or when arriving at the target tissue. For example, the linker may be a peptide having a length of 2 to 50 amino acids, such as a dipeptide, or an organic compound, such as succinimidyl 4-(N-maleimidomethyl)cyclohexane-1-carboxylate (SMCC).

It is preferred that the antibody is covalently linked to the therapeutically active substance of (a) or a chelator thereof or the diagnostic compound of (b) or a chelator thereof, optionally via a linker.

A linker may include one conjugating component or may include multiple components.

For example, the linker may include a spacer, which is a moiety that extends the drug linkage to avoid, for example, shielding the active site of the antibody or improving the solubility of the ADC. Other examples of components of linkers include a stretcher unit and an amino acid unit.

Two methods are commonly used for conjugating drugs to antibodies: alkylation of reduced interchain cysteine disulfides through an enzymatically non-cleavable maleimido or simple and cleavable disulfide linker, and acylation of lysines by cleavable linear amino acids.

In one aspect, a linker covalently attaches an antibody to a therapeutically active substance. The same applies to a diagnostic compound. An ADC is prepared using a linker having reactive functionality for binding to the antibody and the therapeutically active substance or diagnostic compound. For example, a cysteine thiol, or an amine, e.g., N-terminus or amino acid side chain such as lysine, of the antibody may form a bond with a functional group of the linker.

In one embodiment, a linker has a functionality that is capable of reacting with a free cysteine present on an antibody to form a covalent bond. Nonlimiting exemplary such reactive functionalities include maleimide, haloacetamides, α-haloacetyl, activated esters such as succinimide esters, 4-nitrophenyl esters, pentafluorophenyl esters, tetrafluorophenyl esters, anhydrides, acid chlorides, sulfonyl chlorides, isocyanates, and isothiocyanates.

In some embodiments, a linker has a functionality that is capable of reacting with an electrophilic group present on an antibody. Exemplary such electrophilic groups include, but are not limited to, aldehyde and ketone carbonyl groups. In some embodiments, a heteroatom of the reactive functionality of the linker can react with an electrophilic group on an antibody and form a covalent bond to an antibody unit. Nonlimiting exemplary such reactive functionalities include, but are not limited to, hydrazide, oxime, amino, hydrazine, thiosemicarbazone, hydrazine carboxylate, and arylhydrazide.

Suitable linkers include, for example, cleavable and non-cleavable linkers. A linker may be a “cleavable linker”, facilitating release of a drug. Nonlimiting exemplary cleavable linkers include acid-labile linkers (e.g., comprising hydrazone), protease-sensitive (e.g., peptidase-sensitive) linkers, glycosylase-sensitive (e.g. glucuronidase-sensitive) linkers, photolabile linkers, or disulfide-containing linkers. A cleavable linker is typically susceptible to cleavage under intracellular conditions. Suitable cleavable linkers include, for example, a peptide linker cleavable by an intracellular protease, such as lysosomal protease or an endosomal protease. In exemplary embodiments, the linker can be a dipeptide linker, such as a valine-citrulline (val-cit or “VC”), a phenylalanine-lysine (phe-lys) or a valine-alanine (val-ala or “VA”) linker.

For example, a PEG₈-VA linker may be used, as shown in the Examples for the ADC conjugate to tesirine.

For example, the linker may contain a maleimide group for attachment to the antibody, a PEG₈ linker, and a cleavable val-ala moiety, bound to the therapeutically active substance. For example, the therapeutically active substance is a pyrrolobenzodiazepine (PBD) such as SG3199, a maytansinoid such as DM4 or an auristatin, such as MMAE.

For example, the linker may contain a cleavable betaglucuronide moiety, such as MC-betaglucuronide in the examples, bound to the therapeutically active substance. For example, the therapeutically active substance is a PDB such as SG3199, a maytansinoid such as DM4 or an auristatin, such as MMAE.

For example, the linker may contain a cleavable val-cit (“VC”) moiety, such as MC-VC-PABC in the examples, bound to the therapeutically active substance. For example, the therapeutically active substance is a PDB such as SG3199, a maytansinoid such as DM4 or an auristatin, such as MMAE.

For example, the linker may contain a cleavable sulfo-SPDB moiety, such as sulfo-SPDB in the examples, bound to the therapeutically active substance. For example, the therapeutically active substance is a PDB such as SG3199, a maytansinoid such as DM4 or an auristatin, such as MMAE.

Linkers are preferably stable extracellularly in a sufficient manner to be therapeutically effective. Before transport or delivery into a cell, the ADC is preferably stable and remains intact, i.e. the antibody remains conjugated to the drug moiety. Linkers that are stable outside the target cell may be cleaved at some efficacious rate once inside the cell. Thus, an effective linker will: (i) maintain the specific binding properties of the antibody; (ii) allow delivery, e.g., intracellular delivery, of the therapeutically active substance; and (iii) maintain the therapeutic effect, e.g., cytotoxic effect, of a therapeutically active substance.

In one embodiment, the linker is cleavable under intracellular conditions, such that cleavage of the linker sufficiently releases the drug from the antibody in the intracellular environment to be therapeutically effective. In some embodiments, the cleavable linker is pH-sensitive, i.e., sensitive to hydrolysis at certain pH values. Typically, the pH-sensitive linker is hydrolyzable under acidic conditions. For example, an acid-labile linker that is hydrolyzable in the lysosome (e.g., a hydrazone, semicarbazone, thiosemicarbazone, cis-aconitic amide, orthoester, acetal, ketal, or the like) can be used. Such linkers are relatively stable under neutral pH conditions, such as those in the blood, but are unstable at below pH 5.5 or 5.0, the approximate pH of the lysosome. In certain embodiments, the hydrolyzable linker is a hydrazone-containing linker.

In other embodiments, the linker is cleavable under reducing conditions (e.g., a disulfide linker). A variety of disulfide linkers are known in the art, including, for example, those that can be formed using SATA (N-succinimidyl-5-acetylthioacetate), SPDP (N-succinimidyl-3-(2-pyridyldithio) propionate), SPDB (N-succinimidyl-3-(2-pyridyldithio) butyrate) and SMPT (N-succinimidyloxycarbonyl-alpha-methyl-alpha-(2-pyridyl-dithio) toluene).

In some embodiments, the linker is cleavable by a cleaving agent, e.g., an enzyme, that is present in the intracellular environment (e.g., within a lysosome or endosome or caveolae). The linker can be, e.g., a peptidyl linker that is cleaved by an intracellular peptidase or protease enzyme, including, but not limited to, a lysosomal or endosomal protease. In some embodiments, the peptidyl linker is at least two amino acids long or at least three amino acids long. Cleaving agents can include cathepsins B and D and plasmin, all of which are known to hydrolyze dipeptide drug derivatives resulting in the release of active drug inside target cells. Most typical are peptidyl linkers that are cleavable by enzymes that are present in L1CAM-expressing cells. In a specific embodiment, the peptidyl linker cleavable by an intracellular protease is a val-cit linker or a val-ala linker. One advantage of using intracellular proteolytic release of the therapeutic agent is that the agent is typically attenuated when conjugated and the serum stabilities of the conjugates are typically high.

In other embodiments, the linker is a malonate linker, a maleimidobenzoyl linker, or a 3′-N-amide analogue.

In yet other embodiments, the linker unit is not cleavable and the drug is released, for example, by antibody degradation.

In some embodiments, the linker is a substantially hydrophilic linker (e.g., PEG₈, PEG₄-Mal and sulfo-SPDB). A hydrophilic linker may be used to reduce the extent to which the drug may be pumped out of resistant cancer cells through MDR (multiple drug resistance) or functionally similar transporters, or to improve the pharmacokinetic properties of an ADC.

In other embodiments, upon cleavage, the linker functions to directly or indirectly inhibit cell growth and/or cell proliferation. For example, in some embodiments, the linker, upon cleavage, can function as an intercalating agent, thereby inhibiting macromolecular biosynthesis (e.g. DNA replication, RNA transcription, and/or protein synthesis).

In other embodiments, the linker is designed to facilitate bystander killing (the killing of neighboring cells) through diffusion of the linker-drug and/or the drug alone to neighboring cells. In other embodiments, the linker promotes cellular internalization.

The presence of a sterically hindered disulfide can increase the stability of a particular disulfide bond, enhancing the potency of the ADC. Thus, in one embodiment, the linker includes a sterically hindered disulfide linkage. A sterically hindered disulfide refers to a disulfide bond present within a particular molecular environment, wherein the environment is characterized by a particular spatial arrangement or orientation of atoms, typically within the same molecule or compound, which prevents or at least partially inhibits the reduction of the disulfide bond. Thus, the presence of bulky (or sterically hindering) chemical moieties and/or bulky amino acid side chains proximal to the disulfide bond prevents or at least partially inhibits the disulfide bond from potential interactions that would result in the reduction of the disulfide bond.

In a preferred embodiment of any of the aspects of the invention, the antibody is
(a) linked to a therapeutically active substance,
   preferably to
   a chemotherapeutic compound,
   a cytotoxic compound,
   a cytostatic compound,
   a cytokine,
   a nanoparticle,
   a radioisotope, or
   an oncolytic virus,
   and/or
(b) linked to a diagnostic compound, preferably selected from a radioisotope, a chemoluminescent compound, a fluorescent compound, a dye or an enzyme,

In a preferred embodiment, the therapeutically active substance of (a) and/or the diagnostic compound of (b) is selected from a radioisotope, a chemotherapeutic compound, a cytotoxic compound, and/or a cytostatic compound, and/or wherein the antibody is covalently linked to the therapeutically active substance of (a) or a chelator thereof or the diagnostic compound of (b) or a chelator thereof, optionally via a linker.

Preferably, the antibody according to any of the aspects of the invention may further be linked to one or more molecules in order to form an antibody conjugate, in particular to a therapeutically active substance and/or a diagnostic compound.

The preferred embodiments of any of the aspects of the invention also apply to the antibody conjugates herein.

Accordingly, preferably, the antibody portion of the antibody conjugate comprises:
(a) a heavy chain variable region (VH) comprising a VH CDR1 comprising the amino acid sequence of GYSITSDYTWN (SEQ ID NO: 9), a VH CDR2 comprising the amino acid sequence of YISYSGSYSYNPSLKS (SEQ ID NO: 11), and a VH CDR3 comprising the amino acid sequence of SFSYSYGFAY (SEQ ID NO: 14); and
(b) a light chain variable region (VL) comprising a VL CDR1 comprising the amino acid sequence of KASQDVSSAVA (SEQ ID NO: 4), a VL CDR2 comprising the amino acid sequence of SASYRYT (SEQ ID NO: 5), and a VL CDR3 comprising the amino acid sequence of QQHYSTPWT (SEQ ID NO: 6).

Accordingly, preferably, the antibody portion of the antibody conjugate comprises a heavy chain variable region sequence comprising or consisting of the amino acid sequence of SEQ ID NO: 30 and/or comprises a light chain variable region sequence comprising or consisting of the amino acid sequence of SEQ ID NO: 20. Further, preferably, the antibody portion of the antibody conjugate comprises a heavy chain variable region sequence comprising or consisting of the amino acid sequence of SEQ ID NO: 30 and comprises a light chain variable region sequence comprising or consisting of the amino acid sequence of SEQ ID NO: 20.

Preferably, the antibody portion of the antibody conjugate comprises a heavy chain sequence comprising or consisting of an amino acid sequence of SEQ ID NO: 37 and/or a light chain sequence comprising or consisting of an amino acid sequence of SEQ ID NO: 38. In a yet further preferred embodiment, the antibody portion of the antibody conjugate comprises a heavy chain sequence comprising or consisting of an amino acid sequence of SEQ ID NO: 37 and a light chain sequence comprising or consisting of an amino acid sequence of SEQ ID NO: 38.

In another further preferred embodiment, the antibody portion of the antibody conjugate consists of a heavy chain sequence comprising or consisting of an amino acid sequence of SEQ ID NO: 37 and/or a light chain sequence comprising or consisting of an amino acid sequence of SEQ ID NO: 38. In another further preferred embodiment, the antibody portion of the antibody conjugate consists of a heavy chain sequence comprising or consisting of an amino acid sequence of SEQ ID NO: 37 and a light chain sequence comprising or consisting of an amino acid sequence of SEQ ID NO: 38.

Preferably, the antibody portion of the antibody conjugate comprises a heavy chain sequence comprising or consisting of an amino acid sequence of SEQ ID NO: 84 and/or a light chain sequence comprising or consisting of an amino acid sequence of SEQ ID NO: 38. In a yet further preferred embodiment, the antibody portion of the antibody conjugate comprises a heavy chain sequence comprising or consisting of an amino acid sequence of SEQ ID NO: 84 and a light chain sequence comprising or consisting of an amino acid sequence of SEQ ID NO: 38.

In another further preferred embodiment, the antibody portion of the antibody conjugate consists of a heavy chain sequence comprising or consisting of an amino acid sequence of SEQ ID NO: 84 and/or a light chain sequence comprising or consisting of an amino acid sequence of SEQ ID NO: 38. In another further preferred embodiment, the antibody portion of the antibody conjugate consists of a heavy chain sequence comprising or consisting of an amino acid sequence of SEQ ID NO: 84 and a light chain sequence comprising or consisting of an amino acid sequence of SEQ ID NO: 38.

In one preferred embodiment, the antibody of any of the aspects is linked to at least one therapeutically active substance via a linker. Such compound is also referred to as “Antibody-Drug Conjugate”, “antibody conjugate”, “antibody drug conjugate” or “ADC”.

Antibodies of the invention described herein may be conjugated to a drug moiety to form an anti-L1CAM Antibody Drug Conjugate (ADC). Antibody-drug conjugates (ADCs) may increase the therapeutic efficacy of antibodies in treating disease, e.g., cancer, due to the ability of the ADC to selectively deliver one or more therapeutically active substance moiety(s) to target tissues or cells, e.g., L1CAM expressing tumors or L1CAM expressing cells. Thus, in certain embodiments, the disclosure provides anti-L1CAM ADCs for therapeutic use, e.g., treatment of cancer.

The terms “therapeutically active substance”, “therapeutically active substance moiety”, “drug,” “agent,” and “drug moiety” are used interchangeably herein.

The terms “linked” and “conjugated” are also used interchangeably herein and indicate that the antibody and moiety are covalently linked.

In some embodiments, the ADC, or antibody of the invention linked to at least one therapeutically active substance via a linker, has the following formula (formula I):

Ab-(L-D)n (I)

wherein Ab is an antibody of any of the aspects of the invention described herein, and (L-D) is a Linker-Drug moiety. The Linker-Drug moiety is made of L-which is a Linker, and -D, which is a therapeutically active substance moiety (or drug moiety) having, for example, cytostatic, cytotoxic, or otherwise therapeutic activity against a target cell, e.g., a cell expressing L1CAM; and n is an integer from 1 to 20. In some embodiments, n ranges from 1 to 8, 1 to 7, 1 to 6, 1 to 5, 1 to 4, 1 to 3, 1 to 2, or is 1.

Examples of therapeutically active substances that may be used in ADCs, i.e., therapeutically active substances that may be conjugated to the antibodies of the invention include mitotic inhibitors, antitumor antibiotics, immunomodulating agents, gene therapy vectors, alkylating agents, antiangiogenic agents, antimetabolites, boron-containing agents, chemoprotective agents, hormone agents, glucocorticoids, photoactive therapeutic agents, oligonucleotides, radioactive isotopes, radiosensitizers, topoisomerase inhibitors, tyrosine kinase inhibitors, and combinations thereof.

In one embodiment, the therapeutically active substance is selected from the group consisting of a DNA damaging agent, an anti-apoptotic agent, a mitotic inhibitor, an anti-tumor antibiotic, an immunomodulating agent, a nucleic acid for gene therapy, an anti-angiogenic agent, an anti-metabolite, a boron-containing agent, a chemoprotective agent, a hormone agent, an anti-hormone agent, a corticosteroid, a photoactive therapeutic agent, an oligonucleotide, a radioisotope, a radiosensitizer, a topoisomerase inhibitor, and a tyrosine kinase inhibitor.

### Mitotic Inhibitors

In one embodiment, antibodies of the invention may be conjugated to one or more mitotic inhibitor(s) to form an ADC for the treatment of a hyperproliferative disorder, a tumor disease, a disorder associated with neoangiogenesis and/or a disorder associated with aberrant neurogenesis. The term “mitotic inhibitor”, as used herein, refers to a cytotoxic and/or therapeutic agent that blocks mitosis or cell division, a biological process particularly important to cancer cells. A mitotic inhibitor disrupts microtubules such that cell division is prevented, often by affecting microtubule polymerization (e.g., inhibiting microtubule polymerization) or microtubule depolymerization (e.g., stabilizing the microtubule cytoskeleton against depolymerization). Thus, in one embodiment, an antibody of the invention is conjugated to one or more mitotic inhibitor(s) that disrupts microtubule formation by inhibiting tubulin polymerization. In another embodiment, an antibody of the invention is conjugated to one or more mitotic inhibitor(s) that stabilizes the microtubule cytoskeleton from depolymerization. In one embodiment, the mitotic inhibitor used in the ADCs of the invention is Ixempra (ixabepilone). Examples of mitotic inhibitors that may be used in the ADCs of the invention are provided below. Included in the genus of mitotic inhibitors are auristatins and maytansinoids, which are further described below.

### Dolastatins

The antibodies of the invention may be conjugated to at least one dolastatin to form an ADC. Dolastatins are short peptidic compounds isolated from the Indian Ocean sea hare Dolabella auricularia. Examples of dolastatins include dolastatin 10 and dolastatin 15. Dolastatin 15, a seven-subunit depsipeptide derived from Dolabella auricularia, is a potent antimitotic agent structurally related to the antitubulin agent dolastatin 10, a five-subunit peptide obtained from the same organism. Auristatins are synthetic derivatives of dolastatin 10.

### Auristatins

Antibodies of the invention may be conjugated to at least one auristatin. Auristatins represent a group of dolastatin analogues that have generally been shown to possess anticancer activity by interfering with microtubule dynamics and GTP hydrolysis, thereby inhibiting cellular division. For example, Auristatin E is a synthetic analogue of the marine natural product dolastatin 10, a compound that inhibits tubulin polymerization by binding to the same site on tubulin as the anticancer drug vincristine. Dolastatin 10, auristatin PE, and auristatin E are linear peptides having four amino acids, three of which are unique to the dolastatin class of compounds. Exemplary embodiments of the auristatin subclass of mitotic inhibitors include, but are not limited to, monomethyl auristatin D (MMAD or auristatin D derivative), monomethyl auristatin E (MMAE or auristatin E derivative), monomethyl auristatin F (MMAF or auristatin F derivative), auristatin F phenylenediamine (AFP), auristatin EB (AEB), auristatin EFP (AEFP), and 5-benzoylvaleric acid-AE ester (AEVB).

In one embodiment, an antibody of the invention is conjugated to at least one MMAE (monomethyl auristatin E). Monomethyl auristatin E (MMAE) inhibits cell division by blocking the polymerization of tubulin. Because of its toxicity, it also cannot be used as a drug itself. In recent cancer therapy developments, it is linked to an antibody that recognizes a specific marker expressed in cancer cells and directs MMAE to the cancer cells. In one embodiment, the linker linking MMAE to an antibody of the invention is stable in extracellular fluid (i.e., the medium or environment that is external to cells), but is cleaved by cathepsin once the ADC has bound to the specific cancer cell antigen and entered the cancer cell, thus releasing the toxic MMAE and activating the potent anti-mitotic mechanism. In one embodiment, the linker linking MMAE to an antibody of the invention is stable in extracellular fluid (i.e., the medium or environment that is external to cells), but is cleaved by glucuronidase once the ADC has bound to the specific cancer cell antigen and entered the cancer cell, thus releasing the toxic MMAE and activating the potent anti-mitotic mechanism.

### Maytansinoids

The antibodies of the invention may be conjugated to at least one maytansinoid to form an ADC. Maytansinoids are potent antitumor agents that were originally isolated from members of the higher plant families Celastraceae, Rhamnaceae, and Euphorbiaceae, as well as some species of mosses. Evidence suggests that maytansinoids inhibit mitosis by inhibiting polymerization of the microtubule protein tubulin, thereby preventing formation of microtubules. Maytansinoids have been shown to inhibit tumor cell growth in vitro using cell culture models, and in vivo using laboratory animal systems. Moreover, the cytotoxicity of maytansinoids is 1,000-fold greater than conventional chemotherapeutic agents, such as, for example, methotrexate, daunorubicin, and vincristine. Maytansinoids include for example maytansine, maytansinol, and C-3 esters of maytansinol.

Suitable maytansinoids for use in ADCs of the invention can be isolated from natural sources, synthetically produced, or semi-synthetically produced. Moreover, the maytansinoid can be modified in any suitable manner, as long as sufficient cytotoxicity is preserved in the ultimate conjugate molecule. In this regard, maytansinoids lack suitable functional groups to which antibodies can be linked. A linking moiety desirably is utilized to link the maytansinoid to the antibody to form the conjugate, and is described in the Examples.

Representative examples of maytansinoids include, but are not limited, to DM1 (N2′-deacetyl-N2′-(3-mercapto-1-oxopropyl)-maytansine; also referred to as drug maytansinoid 1, DM2, DM3 (N2′-deacetyl-N2′-(4-mercapto-1-oxopentyl)-maytansine), DM4 (4-methyl-4-mercapto-1-oxopentyl)-maytansine), and maytansinol (a synthetic maytansinoid analog).

In one embodiment of the invention, an antibody of the invention is conjugated to at least one DM1. In one embodiment, an antibody of the invention is conjugated to at least one DM2. In one embodiment, an antibody of the invention is conjugated to at least one DM3. In one embodiment, an antibody of the invention is conjugated to at least one DM4.

### Antitumor Antibiotics

An antibody of the invention may be conjugated to one or more antitumor antibiotic(s). As used herein, the term “antitumor antibiotic” means an antineoplastic drug that blocks cell growth by interfering with DNA and is made from a microorganism. Often, antitumor antibiotics either break up DNA strands or slow down or stop DNA synthesis. Examples of antitumor antibiotics that may be included in the ADCs include, but are not limited to, actinomycines (e.g., pyrrolo[2,1-c][1,4]benzodiazepines), anthracyclines, calicheamicins, and duocarmycins. In addition to the foregoing, additional antitumor antibiotics that may be used include bleomycin, mitomycin, and plicamycin (also known as mithramycin).

### Immunomodulating Agents

In one embodiment, an antibody of the invention may be conjugated to at least one immunomodulating agent. As used herein, the term “immunomodulating agent” refers to an agent that can stimulate or modify an immune response. In one embodiment, an immunomodulating agent is an immunostimulator which enhances a subject's immune response. In another embodiment, an immunomodulating agent is an immunosuppressant which prevents or decreases a subject's immune response. An immunomodulating agent may modulate myeloid cells (monocytes, macrophages, dendritic cells, megakaryocytes and granulocytes) or lymphoid cells (T cells, B cells and natural killer (NK) cells) and any further differentiated cells thereof. Representative examples include, but are not limited to, bacillus calmette-guerin (BCG) and levamisole (Ergamisol). Other examples of immunomodulating agents that may be used in the ADCs include, but are not limited to, cancer vaccines, and cytokines.

### Alkylating Agents

The antibody of the invention may be conjugated to one or more alkylating agent(s). Alkylating agents are a class of antineoplastic compounds that attaches an alkyl group to DNA. Examples of alkylating agents that may be used in the ADCs include, but are not limited to, alkyl sulfonates, ethylenimimes, methylamine derivatives, epoxides, nitrogen mustards, nitrosoureas, triazines and hydrazines.

### DNA Damaging Agents

In one embodiment, an antibody of the invention may be conjugated to one or more DNA damaging agents. The term “DNA damaging agent”, as used herein, refers to an agent which is capable of damaging DNA and is well known to those of ordinary skill in the art. DNA damaging agents include DNA alkylating agents. DNA damaging agents also include indolino-benzodiazepines (IGNs).

In one embodiment, a DNA damaging agent may also include a pyrrolobenzodiazepine (PBD) or pyridinobenzodiazepine (PDD) [26, 27]

For example, SG3199 or VA-SG3199 (tesirine) may be used. SG3199 is the pyrrolobenzodiazepine (PBD) dimer warhead component of antibody-drug conjugate (ADC) payload tesirine.

### Antiangiogenic Agents

In one embodiment, an antibody of the invention described herein is conjugated to at least one antiangiogenic agent. Antiangiogenic agents inhibit the growth of new blood vessels. Antiangiogenic agents exert their effects in a variety of ways. In some embodiments, these agents interfere with the ability of a growth factor to reach its target. For example, vascular endothelial growth factor (VEGF) is one of the primary proteins involved in initiating angiogenesis by binding to particular receptors on a cell surface. Thus, certain antiangiogenic agents, that prevent the interaction of VEGF with its cognate receptor, prevent VEGF from initiating angiogenesis. In other embodiments, these agents interfere with intracellular signalling cascades. For example, once a particular receptor on a cell surface has been triggered, a cascade of other chemical signals is initiated to promote the growth of blood vessels. Thus, certain enzymes, for example, some tyrosine kinases, that are known to facilitate intracellular signaling cascades that contribute to, for example, cell proliferation, are targets for cancer treatment. In other embodiments, these agents interfere with intercellular signaling cascades. Yet, in other embodiments, these agents disable specific targets that activate and promote cell growth or by directly interfering with the growth of blood vessel cells. Angiogenesis inhibitory properties have been discovered in more than 300 substances with numerous direct and indirect inhibitory effects. Representative examples of antiangiogenic agents that may be used in the ADCs include, but are not limited to, angiostatin, ABX EGF, C1-1033, PKI-166, EGF vaccine, EKB-569, GW2016, ICR-62, EMD 55900, CP358, PD153035, AG1478, IMC-C225 (Erbitux), ZD1839 (Iressa), OSI-774, Erlotinib (tarceva), arrestin, endostatin, BAY 12-9566 and fluorouracil or doxorubicin, canstatin, carboxyamidotriozole and with paclitaxel, EMD121974, S-24, vitaxin, dimethylxanthenone acetic acid, IM862, Interleukin-12, Interleukin-2, NM-3, HuMV833, PTK787, RhuMab, angiozyme (ribozyme), IMC-1C11, Neovastat, marimstat, prinomastat, BMS-275291,COL-3, MM1270, SU101, SU6668, SU11248, SU5416, with paclitaxel, with gemcitabine and cisplatin, and with irinotecan and cisplatin and with radiation, tecogalan, temozolomide and PEG interferon α2b, tetrathiomolybdate, TNP-470, thalidomide, CC-5013 and with taxotere, tumstatin, 2-methoxyestradiol, VEGF trap, mTOR inhibitors (deforolimus, everolimus (Afinitor, Novartis Pharmaceutical Corporation), and temsirolimus (Torisel, Pfizer, Inc.)), tyrosine kinase inhibitors (e.g., erlotinib (Tarceva, Genentech, Inc.), imatinib (Gleevec, Novartis Pharmaceutical Corporation), gefitinib (Iressa, AstraZeneca Pharmaceuticals), dasatinib (Sprycel, Brystol-Myers Squibb), sunitinib (Sutent, Pfizer, Inc.), nilotinib (Tasigna, Novartis Pharmaceutical Corporation), lapatinib (Tykerb, GlaxoSmithKline Pharmaceuticals), sorafenib (Nexavar, Bayer and Onyx), phosphoinositide 3-kinases (PI3K).

### Antimetabolites

The antibody of the invention may be conjugated to at least one antimetabolite. Antimetabolites are types of chemotherapy treatments that are very similar to normal substances within the cell. When the cells incorporate an antimetabolite into the cellular metabolism, the result is negative for the cell, e.g., the cell is unable to divide. Antimetabolites are classified according to the substances with which they interfere. Examples of antimetabolites that may be used in the ADCs include, but are not limited to, a folic acid antagonist (e.g., methotrexate), a pyrimidine antagonist (e.g., 5-Fluorouracil, Foxuridine, Cytarabine, Capecitabine, and Gemcitabine), a purine antagonist (e.g., 6-Mercaptopurine and 6-Thioguanine) and an adenosine deaminase inhibitor (e.g., Cladribine, Fludarabine, Nelarabine and Pentostatin.

### Boron-Containing Agents

The antibody of the invention may be conjugated to at least one boron containing agent. Boron-containing agents comprise a class of cancer therapeutic compounds which interfere with cell proliferation. Representative examples of boron containing agents include, but are not limited, to borophycin and bortezomib.

### Chemoprotective Agents

The antibodies of the invention may be conjugated to at least one chemoprotective agent.

Chemoprotective drugs are a class of compounds, which help protect the body against specific toxic effects of chemotherapy. Chemoprotective agents may be administered with various chemotherapies in order to protect healthy cells from the toxic effects of chemotherapy drugs, while simultaneously allowing the cancer cells to be treated with the administered chemotherapeutic. Representative chemoprotective agents include, but are not limited to amifostine, which is used to reduce renal toxicity associated with cumulative doses of cisplatin, dexrazoxane, for the treatment of extravasation caused by the administration of anthracycline, and for the treatment of cardiac-related complications caused by the administration of the antitumor antibiotic doxorubicin, and mesna (Mesnex, Bristol-Myers Squibb), which is used to prevent hemorrhagic cystitis during chemotherapy treatment with ifocfamide.

### Photoactive Therapeutic Agents

The antibodies of the invention may be conjugated to at least one photoactive therapeutic agent. Photoactive therapeutic agents include compounds that can be deployed to kill treated cells upon exposure to electromagnetic radiation of a particular wavelength. Therapeutically relevant compounds absorb electromagnetic radiation at wavelengths which penetrate tissue. In preferred embodiments, the compound is administered in a non-toxic form that is capable of producing a photochemical effect that is toxic to cells or tissue upon sufficient activation. In other preferred embodiments, these compounds are retained by cancerous tissue and are readily cleared from normal tissues. Non-limiting examples include various chromagens and dyes.

In a preferred embodiment, the mitotic inhibitor is selected from a maytansinoid and an auristatin.

Maytansinoid DM4 and auristatin MMAE were successfully used in ADCs of the invention in the Examples.

In another preferred embodiment, the DNA damaging agent is selected from a pyrrolobenzodiazepine (PBD) and a pyridinobenzodiazepine (PDD).

In the Examples, DNA damaging agent VA-SG3199 (tesirine) was successfully used in an ADC of the invention.

In another preferred embodiment, the linker is a non-cleavable linker.

In another preferred embodiment, the linker is a cleavable linker.

In yet another preferred embodiment, the therapeutically active substance is selected from monomethyl auristatin E (MMAE), 4-methyl-4-mercapto-1-oxopentyl)-maytansine (DM4), and VA-SG3199 (tesirine).

As described above, any of the antibodies of the invention described herein may be used for the ADCs.

In the Examples, ADCs comprising the AFF4-WT antibody were prepared and were shown to be effective in in vitro and/or in vivo tumor models.

Accordingly, it is particularly preferred to provide an ADC comprising an antibody comprising the CDRH1, CDRH2, CDRH3, CDRL1, CDRL2, and/or CDRL3 sequences of the AFF4 antibody, the VH and/or VL domain sequences of the AFF4 or AFF4-WT antibody and/or the heavy chain and/or light chain sequences of an AFF4 or AFF4-WT antibody.

Preferably, an antibody of the invention linked to at least one therapeutically active substance via a linker, or ADC, is provided comprising the CDRH1, CDRH2, CDRH3, CDRL1, CDRL2, and/or CDRL3 sequences of the AFF4 antibody or the VH and/or VL domain sequences of the AFF4 antibody, and wherein the antibody further comprises a human IgG1, IgG2, IgG3 or IgG4 constant region.

In one embodiment of the antibody of the invention linked to at least one therapeutically active substance via a linker, or ADC of the invention, the antibody further comprises a human IgG1 constant region.

For example, the human IgG constant region may be the wildtype human IgG constant region. Alternatively, the human IgG constant region may be the wildtype human IgG constant region containing 1 to 10 mutations (including 1, 2, 3, 4, 5, 6, 7, 8, 9 or 10 mutations, including any subrange thereof), such as substitutions, deletions and/or insertions, in particular 1 to 10 substitutions (including 1, 2, 3, 4, 5, 6, 7, 8, 9 or 10 substitutions, including any subrange thereof), in the constant region.

Each mutation, independently, may be introduced in one chain of the Fc domain. Alternatively, each mutation, independently, may be introduced symmetrically in both chains of the constant region domain.

In one preferred embodiment, the antibody linked to at least one therapeutically active substance via a linker, or ADC, comprises a heavy chain variable region sequence comprising the amino acid sequence of SEQ ID NO: 30 and/or comprises a light chain variable region sequence comprising the amino acid sequence of SEQ ID NO: 20.

In one preferred embodiment, the antibody linked to at least one therapeutically active substance via a linker, or ADC, comprises a heavy chain variable region sequence comprising the amino acid sequence of SEQ ID NO: 30 and comprises a light chain variable region sequence comprising the amino acid sequence of SEQ ID NO: 20.

In one preferred embodiment, the antibody linked to at least one therapeutically active substance via a linker, or ADC, comprises a heavy chain sequence comprising or consisting of an amino acid sequence of SEQ ID NO: 37 and/or a light chain sequence comprising or consisting of an amino acid sequence of SEQ ID NO: 38.

In one preferred embodiment, the antibody linked to at least one therapeutically active substance via a linker, or ADC, comprises a heavy chain sequence comprising or consisting of an amino acid sequence of SEQ ID NO: 84 and/or a light chain sequence comprising or consisting of an amino acid sequence of SEQ ID NO: 38.

### Nucleic Acid, Vectors and Host Cells

The term “nucleic acid” describes any form of deoxyribonucleic acid (DNA), ribonucleic acid (RNA) or artificial nucleic acid known to the person skilled in the art.

Nucleotide sequences coding for antibodies described herein, and modified versions of these antibodies can be determined using methods well known in the art, i.e., nucleotide codons known to encode particular amino acids are assembled in such a way to generate a nucleic acid that encodes the antibody. Such a polynucleotide encoding the antibody can be assembled from chemically synthesized oligonucleotides, which, briefly, involves the synthesis of overlapping oligonucleotides containing portions of the sequence encoding the antibody, annealing and ligating of those oligonucleotides, and then amplification of the ligated oligonucleotides by PCR.

When a clone containing a nucleic acid encoding a particular antibody is not available, but the sequence of the antibody molecule is known, a nucleic acid encoding the immunoglobulin can be chemically synthesized or obtained from a suitable source (e.g., an antibody cDNA library or a cDNA library generated from, or nucleic acid, preferably poly A+ RNA, isolated from, any tissue or cells expressing the antibody, such as hybridoma cells selected to express an antibody described herein) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR can then be cloned into replicable cloning vectors using any method well known in the art.

DNA coding for the antibodies of the invention described herein can be readily isolated and sequenced using conventional procedures (e.g., by using oligonucleotide probes that are capable of binding specifically to genes encoding the heavy and light chains of the antibodies). Hybridoma cells can serve as a source of such DNA. Once isolated, the DNA can be placed into expression vectors, which are then transfected into host cells such as E. coli cells, simian COS cells, Chinese hamster ovary (CHO) cells (e.g., CHO cells from the CHO GS System™ (Lonza)), or myeloma cells that do not otherwise produce immunoglobulin protein, to obtain the synthesis of the antibodies in the recombinant host cells.

To generate whole antibodies, PCR primers including VH or VL nucleotide sequences, a restriction site, and a flanking sequence to protect the restriction site can be used to amplify the VH or VL sequences in scFv clones or other clones. Utilizing cloning techniques known to those of skill in the art, the PCR amplified VH domains can be cloned into vectors expressing a heavy chain constant region, e.g., the human IgG1 constant region, the human IgG4 constant region, the human IgG2 constant region or the human IgG3 constant region, and the PCR amplified VL domains can be cloned into vectors expressing a light chain constant region, e.g., human kappa or lambda constant regions. In certain embodiments, the vectors for expressing the VH or VL domains comprise a promoter, a secretion signal, a cloning site for the variable region, constant domains, and a selection marker such as neomycin. The VH and VL domains can also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art. Alternatively, the vectors expressing the heavy chain or light chain may be transfected into separate cells and the antibodies may be assembled after recovery. Further, alternatively, the nucleic acid(s) encoding the antibodies of the invention may be expressed using a single vector for expression. Such vector may encode the antibody of the invention as a single molecule, such as an scFv, or may encode two or more polypeptides, which can be assembled into an antibody of the invention.

The DNA also can be modified, for example, by substituting the coding sequence for human heavy and light chain constant domains in place of the murine sequences, or by covalently joining to the immunoglobulin coding sequence all or part of the coding sequence for a non-immunoglobulin polypeptide.

Site-directed or high-density mutagenesis of the variable region or other mutagenesis methods can be used to optimize specificity, affinity, etc. of a monoclonal antibody. Especially, affinity maturation strategies and chain shuffling strategies [28] are known in the art and can be employed to generate high affinity human antibodies.

In another aspect, the present invention also relates to a nucleic acid
(i) coding for an antibody that specifically binds to human L1CAM according to the any of the aspects of the invention, and/or
(ii) encoding at least one VH or HC and/or VL or LC of an antibody that specifically binds to human L1CAM according to any of the aspects of the invention, and/or
(iii) encoding the sequence according to SEQ ID NO: 30 and/or according to SEQ ID NO: 20, and/or
(iv) comprising sequences encoding the complementarity determining region sequences of an antibody that specifically binds to human L1CAM according to any of the aspects of the invention.

In this context, all features described above for the antibodies of any of the aspects of the invention, where applicable, also apply to the nucleic acid of the invention, such as the features related to the antibody this nucleic acid codes for or such an antibody's specific binding.

Preferably, the nucleic acid is part of a vector.

Generally, vectors (also expression vectors) are plasmids which are used to introduce a desired nucleic acid sequence, such as a gene, into a target cell, resulting in the transcription and translation of the protein encoded by the nucleic acid sequence, e.g. the chimeric antigen receptor, the antibody or the binding molecule. Therefore, the expression vector in general comprises regulatory sequences, such as promoter and enhancer regions, as well as a polyadenylation site in order to direct efficient transcription of the nucleic acid sequence on the expression vector. The expression vector may further comprise additional necessary or useful regions, such as a selectable marker for selection in eukaryotic or prokaryotic cells, a purification tag for the purification of the resulting protein, a multiple cloning site or an origin of replication.

Usually, the expression vector may be a viral or a non-viral vector. In general, various kinds of viral vectors, such as retroviral vectors, e.g. lentiviral or adenoviral vectors, or plasmids may be used.

It is preferred that the nucleic acid is part of a vector.

Such a vector comprising the nucleic acid of the invention may further be introduced in a host cell.

Methods for introducing such a vector into a host cell are well known to the person skilled in the art, such as any known transfection method, e.g. any nonviral transfection method (e.g. chemical-based, non-chemical or particle-based) or any virus-based transfection method. Examples of suitable methods are transfection methods based on calcium phosphate precipitation, lipofection, cationic polymers, Fugene, Dendrimer, nanoparticles, microinjection, cell squeezing, electroporation, particle gun (also known as gene gun), magnet assisted transfection, optical transfection, protoplast fusion, impalafection, hydrodynamic delivery, sonoporation, transferrin-based infection, antibody-mediated transfection or virus-based transfection (e.g. based on adenoviral or lentiviral vectors).

In another aspect, the present invention relates to a host cell comprising a nucleic acid according to the present invention.

Suitable host cells are well known to the person skilled in the art, such as mammalian cells (such as human, mouse, rat or hamster cells), insect cells, bacterial cells or yeast cells. Such host cell may comprise a nucleic acid of the present invention e.g. integrated in its genome or in a vector. Methods for introducing such nucleic acid in the host cell are described above and further well known to the person skilled in the art.

### Compositions Comprising Antibody of the Invention

In another aspect, the present invention relates to a pharmaceutical composition, comprising an antibody of the invention, or a nucleic acid of the invention, or a host cell of the invention, and optionally one or more pharmaceutically acceptable carriers.

The content of the antibody, the nucleic acid or the host cell in the pharmaceutical composition is not limited as far as it is useful for treatment or prevention, but preferably contains 0.0000001-10% by weight per total composition.

Further, the antibody, the nucleic acid or the host cell described herein are preferably employed in one or more pharmaceutically acceptable carrier(s).

The term “carrier” describes any molecule which improves the selectivity, effectiveness and/or safety of administration of the antibody, the nucleic acid or the host cell to a human or animal body, such as by continuous or triggered release or by allowing membrane permeation of the antibody, nucleic acid or host cell.

A carrier is further considered as being pharmaceutically acceptable, when it does not have any or not substantially adverse unwanted effects on the human or animal body, e.g. it is considered generally safe, nontoxic and/or does not cause unwanted biological side reactions. Suitable pharmaceutically acceptable carriers are well known to the person skilled in the art. The choice of carrier may depend upon route of administration and concentration of the active agent(s) and the carrier may be in the form of a lyophilised composition or an aqueous solution. Generally, an appropriate amount of a pharmaceutically acceptable salt is used in the carrier to render the composition isotonic. Examples of the carrier include but are not limited to saline, Ringer's solution and dextrose solution. Preferably, acceptable excipients, carriers, or stabilisers are non-toxic at the dosages and concentrations employed, including buffers such as citrate, phosphate, and other organic acids; salt-forming counter-ions, e.g. sodium and potassium; low molecular weight (<10 amino acid residues) polypeptides; proteins, e.g. serum albumin, or gelatine; hydrophilic polymers, e.g. polyvinylpyrrolidone; amino acids such as histidine, glutamine, lysine, asparagine, arginine, or glycine; carbohydrates including glucose, mannose, or dextrins; monosaccharides; disaccharides; other sugars, e.g. sucrose, mannitol, trehalose or sorbitol; chelating agents, e.g. EDTA; non-ionic surfactants, e.g. Tween, Pluronics or polyethylene glycol; antioxidants including methionine, ascorbic acid and tocopherol; and/or preservatives, e.g. octadecyldimethylbenzyl ammonium chloride; hexamethonium chloride; benzalkonium chloride, benzethonium chloride; phenol, butyl or benzyl alcohol; alkyl parabens, e.g. methyl or propyl paraben; catechol; resorcinol; cyclohexanol; 3-pentanol; and m-cresol). Suitable carriers and their formulations are described in greater detail in Remington's Pharmaceutical Sciences, 17th ed., 1985, Mack Publishing Co.

### Therapeutic and Diagnostic Uses and Applications

In another aspect, the present invention relates to an antibody of the present invention, or a nucleic acid of the present invention, or a host cell of the present invention, or a pharmaceutical composition of the present invention, for use as a medicament or as a diagnostic agent.

The term “medicament” is a substance applicable to cure, treat or prevent a disease. Similarly, the term “diagnostic agent” is a substance applicable to diagnose a disease, i.e. to determine, whether a human or animal body have a specific disease or condition.

In another aspect, the present invention relates to an antibody of the present invention, or a nucleic acid of the present invention, or a host cell of the present invention, or a pharmaceutical composition of the present invention, for use in treating or preventing a hyperproliferative disorder, a tumor disease, a disorder associated with neoangiogenesis and/or a disorder associated with aberrant neurogenesis.

The term “treating” describes any form of improving the health status of a human or animal body with respect to a disease or a condition. This may comprise a lessening of symptoms, slowing progression, a slight improvement, but also the complete cure of the human or animal body of the disease or condition. Similarly, the term “preventing” describes keeping the health status of a human or animal body from suffering from a disease or a condition.

The term “hyperproliferative disorder” describes any disease or condition related to hyperproliferative cells (such as tumors in general, cancers, and neoplastic tissue, along with pre-malignant and non-neoplastic or non-malignant hyperproliferative disorders), i.e. cells showing an abnormally high rate of cell proliferation. Examples are non-malignant (also non-neoplastic), premalignant or malignant tumors. Examples of premalignant and non-neoplastic or non-malignant hyperproliferative disorders are e.g. myelodysplastic disorders; cervical carcinoma-in-situ; familial intestinal polyposes such as Gardner syndrome; oral leukoplakias; histiocytoses; keloids; hemangiomas; hyperproliferative arterial stenosis, inflammatory arthritis; hyperkeratosis, papulosquamous eruptions including arthritis, and hyperproliferative skin disorder, such as chronic inflammatory skin disease (e.g. psoriasis), as well as virus-induced hyperproliferative diseases such as warts and EBV induced disease (i.e., infectious mononucleosis), scar formation, and the like.

The term “tumor disease” describes any disease or condition related to tumors (also called neoplasms), which may be non-malignant (also non-neoplastic), premalignant or malignant.

In embodiments, the tumor disease is selected from ovarian cancer, breast cancer, endometrial cancer, melanoma and neuroblastoma.

In an embodiment, the antibody of the present invention, or nucleic acid of the present invention, or host cell of the present invention, or pharmaceutical composition of the present invention, is for use in treating or preventing a L1CAM expressing hyperproliferative disorder, a L1CAM expressing tumor disease, a L1CAM expressing disorder associated with neoangiogenesis and/or a L1CAM expressing disorder associated with aberrant neurogenesis.

An “L1CAM expressing hyperproliferative disorder” is understood as hyperproliferative disorder wherein at least part of the hyperproliferative cells express human L1CAM on the cell surface.

An “L1CAM expressing tumor disease” is understood as tumor disease wherein at least part of the tumor cells express human L1CAM on the cell surface.

In embodiments, the L1CAM expressing tumor disease is selected from L1CAM expressing ovarian cancer, L1CAM expressing breast cancer, L1CAM expressing endometrial cancer, L1CAM expressing melanoma and L1CAM expressing neuroblastoma.

An “L1CAM expressing disorder associated with neoangiogenesis” is understood as disorder associated with neoangiogenesis wherein at least part of the cells involved in neoangiogenesis express human L1CAM on the cell surface.

An “L1CAM expressing disorder associated with aberrant neurogenesis” is understood as disorder associated with aberrant neurogenesis wherein at least part of the cells involved in aberrant neurogenesis express human L1CAM on the cell surface.

In another preferred embodiment, the antibody of any of the aspects linked to at least one therapeutically active substance via a linker (also referred to as “Antibody-Drug Conjugate”, “antibody conjugate”, “antibody drug conjugate” or “ADC”) is for use is for use in treating or preventing a hyperproliferative disorder, a tumor disease, a disorder associated with neoangiogenesis, a disorder associated with aberrant neurogenesis, a L1CAM expressing hyperproliferative disorder, a L1CAM expressing tumor disease, a L1CAM expressing disorder associated with neoangiogenesis or a L1CAM expressing disorder associated with aberrant neurogenesis by exhibiting a cytotoxic bystander effect. In embodiments, the linker is a releasable liner.

A cytotoxic bystander effect is understood as effect wherein ADCs endowed with such a bystander effect are taken up and processed by antigen-positive cancer cells in a way that releases a form of the cytotoxic payload, which is freely diffusible to neighboring cells, and thus has the ability to kill those cells independently of their antigen expression. Such ADCs are thus well suited for the treatment of tumors with heterogeneous L1CAM target expression. In embodiments, the linker is a releasable liner. Methods for determining a cytotoxic bystander effect are known in the art. In particular, the assay as described in Example 8 may be used.

“About” is understood to mean the indicated value±10%.

A disorder associated with neoangiogenesis describes any disease or condition related to blood vessel formation in hyperproliferative tissue, such as non-malignant (also non-neoplastic), premalignant or malignant tumors and cancers.

A disorder associated with aberrant neurogenesis describes any disease or condition related to the production of aberrant cells of the nervous system (such as aberrant neurons) by neural stem cells, such as aberrant (hippocampal) neurogenesis.

In another aspect, the present invention relates to an antibody of the present invention, or a nucleic acid of the present invention, or a host cell of the present invention, or a pharmaceutical composition of the present invention, for use in diagnosing a hyperproliferative disorder, a tumor disease, a disorder associated with neoangiogenesis and/or a disorder associated with aberrant neurogenesis.

In another aspect, the present invention relates to the in vitro use of an antibody of the present invention, or a nucleic acid of the present invention, or a host cell of the present invention, or a pharmaceutical composition of the present invention as a diagnostic agent, in particular for diagnosing a hyperproliferative disorder, a tumor disease, a disorder associated with neoangiogenesis and/or a disorder associated with aberrant neurogenesis.

In another aspect, the invention relates to a method of treating or preventing a hyperproliferative disorder, a tumor disease, a disorder associated with neoangiogenesis and/or a disorder associated with aberrant neurogenesis, comprising administering to a patient in need thereof a pharmaceutically effective amount of an antibody of the present invention, or a nucleic acid of the present invention, or a host cell of the present invention, or a pharmaceutical composition of the present invention.

As used herein, the term “pharmaceutically effective amount” in the context of the administration of a therapy to a subject refers to the amount of a therapy that achieves a desired prophylactic or therapeutic effect.

A suitable amount and dosage can be determined by persons skilled in the art. For example, an antibody or pharmaceutical composition described herein may be administered to a subject (e.g., via intravenous injection) at about 0.001 mg/kg, 0.01 mg/kg 0.1 mg/kg, 0.3 mg/kg, 1 mg/kg, 3 mg/kg, 6 mg/kg, or about 10 mg/kg.

Thereby, administration of the antibody, the nucleic acid, or the host cell of the present invention refers to any route of drug administration known to the person skilled in the art, such as intravenous, intraperitoneal, subcutaneous, oral, intranasal or sublingual administration. Suitable dosage regimen are also well known to the person skilled in the art. Preferably, the antibody, the nucleic acid, or the host cell of the present invention is administered in a pharmaceutically effective amount, i.e. in a dose or concentration causing a biological response in the body the antibody, the nucleic acid, or the host cell of the present invention is administered to.

In this context, all features described above for an antibody of any aspect of the invention, where applicable, also apply to the further aspect of the invention, such as the features related to the antibody conjugates, nucleic acid, host cell or pharmaceutical composition.

In general, the disclosure is not limited to the particular methodology, protocols, and reagents described herein because they may vary. Further, the terminology used herein is for the purpose of describing particular embodiments only and is not intended to limit the scope of the present disclosure. As used herein and in the appended claims, the singular forms “a”, “an”, and “the” include plural reference unless the context clearly dictates otherwise. Similarly, the words “comprise”, “contain” and “encompass” are to be interpreted inclusively rather than exclusively.

Unless defined otherwise, all technical and scientific terms and any acronyms used herein have the same meanings as commonly understood by one of ordinary skill in the art in the field of the disclosure. Although any methods and materials similar or equivalent to those described herein can be used in the practice as presented herein, the specific methods, and materials are described herein.

The disclosure is further illustrated by the following figures and examples, although it will be understood that the figures and examples are included merely for purposes of illustration and are not intended to limit the scope of the disclosure unless otherwise specifically indicated.

### REFERENCES

1. Galluzzi L et al.: Trial Watch: Monoclonal antibodies in cancer therapy. Oncoimmunology 1(1):28-37 (2012)
2. Presta LG: Molecular engineering and design of therapeutic antibodies. Curr. Opin. Immunol. 20(4):460-470 (2008)
3. Reichert J M: Marketed therapeutic antibodies compendium. MAbs 2012, 4(3):413-415 (2012)
4. Reichert J M: Antibodies to watch in 2014: mid-year update. mAbs 6(4):799-802 (2014)
5. Reichert J M: Antibody-based therapeutics to watch in 2011. MAbs 3(1):76-99 (2011)
6. Wolterink S et al.: Therapeutic antibodies to human L1CAM: functional characterization and application in a mouse model for ovarian carcinoma. Cancer Res. 70(6):2504-2515 (2010)
7. Doberstein K et al.: Antibody therapy to human L1CAM in a transgenic mouse model blocks local tumor growth but induces EMT. Int. J. Cancer 136(5):E326-339 (2015) 8. Novak-Hofer I et al.: Internalization and degradation of monoclonal antibody chCE7 by human neuroblastoma cells. Int. J. Cancer 57(3):427-432 (1994)
9. Schaefer A W et al.: Activation of the MAPK signal cascade by the neural cell adhesion molecule L1 requires L1 internalization. J. Biol. Chem. 274(53):37965-37973 (1999)
10. Long K E et al.: The role of endocytosis in regulating L1-mediated adhesion. J. Biol. Chem. 276(2):1285-1290 (2001)
11. Schaefer A W et al.: L1 endocytosis is controlled by a phosphorylation-dephosphorylation cycle stimulated by outside-in signaling by L1. J. Cell Biol. 157(7):1223-1232 (2002)
12. Kabat et al.: Unusual distributions of amino acids in complementarity-determining (hypervariable) segments of heavy and light chains of immunoglobulins and their possible roles in specificity of antibody-combining sites. J. Biol. Chem. 252(19), 6609-6616 (1977)
13. Kabat et al.: Sequences of proteins of immunological interest. (1991)
14. Chothia and Lesk AM: Canonical structures for the hypervariable regions of immunoglobulins, J. Mol. Biol. 196(4):901-917 (1987)
15. MacCallum et al.: Antibody-antigen interactions: contact analysis and binding site topography. J. Mol. Biol. 262(5):732-745 (1996)
16. Vazquez-Lombardi R. et al.: Challenges and opportunities for non-antibody scaffold drugs. Drug Discovery Today, 20(10):1271-1283 (2015)
17. Winter, G. and Milstein, C.: Man-made antibodies. Nature, 349:293-299 (1999)
18. Smith P et al.: Mouse model recapitulating human Fcγ receptor structural and functional diversity. Proc. Natl. Acad. Sci. USA 109(16):6181-6186 (2012)
19. Fuchs et al.: Targeting recombinant antibodies to the surface of Escherichia coli. Bio/Technology 9:1370-1372 (1991)
20. Hay et al.: Bacteriophage cloning and Escherichia coli expression of a human IgM Fab. Hum. Antibod. Hybridomas 3:81-85 (1992)
21. Huse et al.: Generation of a large combinatorial library of the immunoglobulin repertoire in phage lambda. Science 246:1275-1281 (1989)
22. Griffiths et al.: Human anti-self antibodies with high specificity from phage display libraries. EMBO J. 12:725-734 (1993)
23. Gouveia et al.: Production and purification of functional truncated soluble forms of human recombinant L1 cell adhesion glycoprotein from Spodoptera frugiperda Sf9 cells. Protein Expr. Purif. 52:182-193 (2007)
24. Epitope Mapping, A practical approach, 248 (Practical Approach Series); Oxford University Press, Editors: Olwyn Westwood and Frank Hay (2001)
25. Oleszewski, M. et al: Characterization of the L1CAM-neurocan binding site. Implications for L1CAM-L1CAM homophilic binding. J. Biol. Chem. 275:34478-34485 (2000).
26. Veillard N. et al.: Pyridinobenzodiazepines (PDDs): A new class of sequence-selective DNA mono-alkylating ADC payloads with low hydrophobicity. Cancer Res 78 (13_Supplement): 736. (2018)
27. Stefano J. E., et al.: Micro- and Mid-Scale Maleimide-Based Conjugation of Cytotoxic Drugs to Antibody Hinge Region Thiols for Tumor Targeting. In: Ducry L. (eds) Antibody-Drug Conjugates. Methods in Molecular Biology (Methods and Protocols), vol 1045. Humana Press, Totowa, NJ. (2013)
28. Marks J. D. et al.: By-passing immunization: building high affinity human antibodies by chain shuffling. Biotechnology 10:779-783 (1992)

### FIGURES

FIG. 1. FIG. 1A shows the analysis of purified OV549.20 mouse IgG2a and chimeric OV549.20 human IgG1 by reducing SDS-PAGE (sodium dodecyl sulfate polyacrylamide gel electrophoresis). The heavy chain and the two forms of the light chain (glycosylated and non-glycosylated) are indicated by arrows. The lanes No. 1 to 3 comprise the following samples: Lane 1: Molecular weight marker; Lane 2: OV549.20 mouse IgG2a; Lane 3: Chimeric OV549.20 human IgG1. FIG. 1B shows the mass spectrometry analysis of DTT-reduced chimeric OV549.20 human IgG1, demonstrating the presence of peaks corresponding to the heavy chain (50798.5 Da) and to the glycosylated (25885.5 Da) and non-glycosylated (23533.2 Da) light chain.

FIG. 2 shows the fluorescence signals detected with an image-based cytometer on JIMT-1, SKOV-3 and Panc-1 cells after incubation with chimeric OV549.20 human IgG1 and a secondary Alexa Fluor 488-labeled goat anti-human IgG antibody. A chimeric human IgG1 isotype control antibody was used for comparison.

FIG. 3 shows the binding of OV549.20 mouse IgG2a to human L1CAM in an ELISA assay and its lack of cross-reactivity with other members of the L1 family. Commercially available antibodies specific for human L1CAM, CHL1, NrCAM, and Neurofascin were used as positive controls to demonstrate successful immobilization of all antigens on the ELISA plates and correct functionality of the detection system. The level of binding was quantified by assessing the Optical Density (OD) at 450 nm wavelength in the ELISA assay.

FIG. 4 shows the dose-dependent induction of antibody-dependent cellular cytotoxicity (ADCC) on Panc-1 pancreatic cancer cells by chimeric OV549.20 human IgG1 and Herceptin. The human IgG1 chimeric version of the previously described antibody L9.3 binding to the first Ig domain of L1CAM was tested in parallel, but did not induce ADCC on Panc-1 cells. Shown are mean values from triplicates±standard deviations. Values were fitted to 4 parameter logistic curves using GraphPad Prism software.

FIG. 5 shows the influence of OV549.20 mouse IgG2a, OV52.24 mouse IgG2a and an isotype control antibody on the proliferation of HCT116, SKOV3, and Panc-1 cells as measured by confluency detection with an Incucyte device. Addition of OV549.20 mouse IgG2a reduced the proliferation of all three cell lines, while OV52.24 mouse IgG2a and the mouse IgG2a isotype control had no influence. Shown are mean values from duplicates±standard deviations.

FIG. 6 shows that chimeric OV549.20 human IgG1 inhibited the migration of HCT116 cells on fibronectin coated plates, while an unrelated chimeric human IgG1 isotype control had no influence on cell migration. Shown are mean values from duplicates±standard deviations.

FIG. 7. FIG. 7A shows the inhibitory effect of OV549.20 mouse IgG2a on intraperitoneal tumor mass and ascites volume in a SKOV3 xenograft model of ovarian cancer in mice. SKOV3 cells were injected intraperitoneally and five days thereafter mice were treated either with 10 mg/kg OV549.20 mouse IgG2a (n=10), 10 mg/kg L9.3 mouse IgG2a, or 10 mg/kg of a mouse IgG2a isotype control (n=10). Treatments were repeated three times per week for a total of six weeks, followed by analysis of tumor mass and ascites volume in the peritoneal cavities. FIG. 7B shows the results of a second SKOV3 xenograft experiment. SKOV3 cells were injected intraperitoneally and five days thereafter mice were treated either with 10 mg/kg OV549.20 mouse IgG2a (n=10) or vehicle (n=10). Treatments were repeated three times per week for a total of six weeks, followed by analysis of tumor mass and ascites volume in the peritoneal cavities.

FIG. 8 shows the inhibitory effect of OV549.20 mouse IgG2a on metastasis formation in an intravenous MDA-MB-231-luc2 mouse xenograft model. Mice (n=15 per group) received intravenous injections (three times per week for five weeks) of OV549.20 mouse IgG2a antibody (10 mg/kg) or vehicle, respectively. Three days after the first administration, all animals were injected intravenously with 5×10⁵ MDA-MB-231-luc2 cells. Body weights of animals were recorded in regular intervals and whole body luminescence imaging was carried out on days 7, 14, 21, and 30 after injection of cells to monitor metastatic colonization of organs in the thoracic region.

FIG. 9 shows the analysis of purified humanized antibody variants H1L1 to H4L2 by reducing SDS-PAGE. For comparison, the parental chimeric OV549.20 human IgG1 antibody was included in the analysis. The heavy chain (HC) and the light chain (LC) are indicated by arrows. In the case of chimeric OV549.20 human IgG1, two forms of the light chain are present, a glycosylated and a non-glycosylated form.

FIG. 10. FIG. 10A shows an analysis of the chimeric OV549.20 human IgG1 antibody and the humanized variant H1L1 on a Tosoh TSKgel Butyl-NPR hydrophobic interaction column. FIG. 10B shows the analysis of the humanized antibody variant H1L1 on a Tosoh TSKgel Butyl-NPR column before (“H1L1 non-stressed”) and after an incubation for twelve days at 40° C. in 20 mM sodium citrate pH 5.5 (“H1L1 stressed at pH 5.5”). FIG. 10C shows the analysis of the antibody variant AFF4 on a Tosoh TSKgel Butyl-NPR column before (“AFF4 non-stressed”) and after an incubation for 14 days at 40° C. in 20 mM sodium citrate pH 5.5 (“AFF4 stressed at pH 5.5”).

FIG. 11 shows the influence of AFF4 and a chimeric human IgG1 isotype control antibody on the proliferation of HCT116, SKOV3, and Panc-1 cells as measured by confluency detection with an Incucyte device. Addition of AFF4 reduced the proliferation of all three cell lines compared to the isotype control antibody. Shown are mean values from duplicates±standard deviations.

FIG. 12 shows that AFF4 inhibited the migration of HCT116 cells on fibronectin coated plates, while a chimeric human IgG1 isotype control antibody had no influence on cell migration. Shown are mean values from duplicates±standard deviations.

FIG. 13 shows the % specific lysis obtained through AFF4-mediated antibody-dependent cellular cytotoxicity on PC-03, Panc-1, HeLa and SKOV3 cancer cell lines. Cancer cells were seeded on xCelligence E-plates and allowed to settle and attach to the plates for 16-24 h. Activated Natural Killer cells from three different donors (CD16 low affinity: FF; medium affinity: V/F; high affinity: VV) were added each at two different effector to target (E:T) cell ratios (5:1 or 10:1) to the cancer cells together with AFF4 at a final concentration of 1 μg/mL. Impedance measurements were recorded over 72 hours with the xCelligence RTCA Analyzer and data were converted to % specific cytolysis.

FIG. 14 shows the structures of the four linker/payloads that were used for conjugation to AFF4-WT. FIG. 14A: VA-SG3199 (MP-PEG₈-VA-PABC-SG3199), CAS Nr.: 1595275-62-9. IUPAC: [4-[(2S)-2-[[(2S)-2-[3-[2-[2-[2-[2-[2-[2-[2-[2-[3-(2,5-dioxopyrrol-1-yl)propanoylamino]ethoxy]ethoxy]ethoxy]ethoxy]ethoxy]ethoxy]ethoxy]ethoxy]propanoylamino]-3-methylbutanoyl]amino]propanoyl]amino]phenyl]methyl (6S,6aS)-3-[5-[[(6aS)-2-methoxy-8-methyl-11-oxo-6a,7-dihydropyrrolo[2,1-c][1,4]benzodiazepin-3-yl]oxy] pentoxy]-6-hydroxy-2-methoxy-8-methyl-11-oxo-6a,7-dihydro-6H-pyrrolo[2,1-c][1,4]benzodiazepine-5-carboxylate; FIG. 14B: VC-MMAE (MC-VC-PABC-MMAE), CAS Nr.: 646502-53-6. IUPAC: [4-[(2S)-5-(carbamoylamino)-2-[[(2S)-2-[6-(2,5-dioxopyrrol-1-yl) hexanoylamino]-3-methylbutanoyl]amino]pentanoyl]amino]phenyl]methyl N-[(2S)-1-[(2S)-1-[[(3R,4S,5S)-1-[(2S)-2-[(1R,2R)-3-[(1S,2R)-1-hydroxy-1-phenylpropan-2-yl]amino]-1-methoxy-2-methyl-3-oxopropyl]pyrrolidin-1-yl]-3-methoxy-5-methyl-1-oxoheptan-4-yl]-methylamino]-3-methyl-1-oxobutan-2-yl]amino]-3-methyl-1-oxobutan-2-yl]-N-methylcarbamate; FIG. 14C: Gluc-MMAE (MC-betaglucuronide-MMAE), CAS Nr.: 1703778-92-0. IUPAC: (2S,3S,4S,5R,6S)-6-[2-[3-[6-(2,5-dioxopyrrol-1-yl) hexanoylamino]propanoylamino]-4-[(2S)-1-[(2S)-1-[[(3R,4S,5S)-1-[(2S)-2-[(1R,2R)-3-[(1S,2R)-1-hydroxy-1-phenylpropan-2-yl]amino]-1-methoxy-2-methyl-3-oxopropyl]pyrrolidin-1-yl]-3-methoxy-5-methyl-1-oxoheptan-4-yl]-methylamino]-3-methyl-1-oxobutan-2-yl]amino]-3-methyl-1-oxobutan-2-yl]-methylcarbamoyl]oxymethyl]phenoxy]-3,4,5-trihydroxyoxane-2-carboxylic acid; FIG. 14D: sulfo-SPDB-DM4, CAS Nr. 1626359-59-8; IUPAC: 4-[5-[(2S)-1-[[(1S,2R,3S,5S,6S,16E,18E,20R,21S)-11-chloro-21-hydroxy-12,20-dimethoxy-2,5,9,16-tetramethyl-8,23-dioxo-4,24-dioxa-9,22-diazatetracyclo[19.3.1.110,14.03,5]hexacosa-10,12,14(26),16,18-pentaen-6-yl]oxy]-1-oxopropan-2-yl]-methylamino]-2-methyl-5-oxopentan-2-yl]disulfanyl]-1-(2,5-dioxopyrrolidin-1-yl)oxy-1-oxobutane-2-sulfonic acid.

FIG. 15 shows the fluorescence signals detected with an image-based cytometer on JIMT-1, OVCAR-3 and MeWo cells after co-incubation with AFF4-WT and Human Fabfluor-pH Red Antibody Labeling Dye for 15 min or 20 h, respectively. A chimeric human IgG1 isotype control antibody was used for comparison. Strongly enhanced fluorescence of AFF4-WT/FabFluor treated cells after 20 h compared to isotype control/Fabfluor treated cells indicates efficient uptake and lysosomal localization of AFF4-WT.

FIG. 16 shows the cytotoxic effects of four AFF4-WT-drug conjugates on three different cancer cell lines. Serial dilutions of AFF4-WT-VC-MMAE, AFF4-WT-Gluc-MMAE, AFF4-WT-VA-SG3199, and AFF4-WT-sulfo-SPDB-DM4, were incubated in duplicates for 6 days with JIMT-1 breast cancer cells, MeWo melanoma cells, and OVCAR-3 ovarian cancer cells, respectively. Cell viability was then assessed by ATP quantification, and % viability of AFF4-WT-drug conjugate treated cells was calculated, using untreated cells and cells incubated in the presence of 10 UM Doxorubicin, respectively, as 100% and 0% viability reference values. Shown are individual values from duplicates. Mean values were fitted to 4 parameter logistic curves using GraphPad Prism software.

FIG. 17. FIG. 17A shows the anti-tumor effects of four AFF4-WT-drug conjugates in a human breast cancer xenograft model in mice. JIMT-1 breast cancer cells were implanted intramammarily and tumors were grown to an average size of approximately 100 mm³. Mice then received single i.v. injections of the indicated AFF4-WT-drug conjugates, or vehicle as control, and tumor growth was monitored over time. FIG. 17B shows the anti-tumor effects of four AFF4-WT-drug conjugates in a human ovarian cancer xenograft model in mice. OVCAR-3 ovarian cancer cells were implanted subcutaneously and tumors were grown to an average size of approximately 140 mm³. Mice then received a first i.v. injection of the indicated AFF4-WT-drug conjugates, or vehicle as control, followed by a second injection after 14 days. Tumor growth was monitored at regular intervals. FIG. 17C shows the anti-tumor effects of four AFF4-WT-drug conjugates in a human melanoma xenograft model in mice. MeWo melanoma cells were implanted subcutaneously and tumors were grown to approximately 140 mm³. Mice then received single i.v. injections of the indicated AFF4-WT-drug conjugates, or vehicle as control, and tumor growth was monitored at regular intervals.

FIG. 18 shows the anti-tumor effects of different doses of AFF4-WT-VC-MMAE in a human breast cancer xenograft model in mice. JIMT-1 breast cancer cells were implanted intramammarily and tumors were grown to an average size of approximately 100 mm³. Mice then received single i.v. injections of the indicated doses of AFF4-WT-VC-MMAE, and tumor growth was monitored over time.

FIG. 19 shows the anti-tumor effects of treatment with AFF4-WT-VC-MMAE in four different L1CAM-expressing patient derived xenograft (PDX) models of human ovarian cancer in mice. PDX tumor fragments were implanted subcutaneously and grown to an average size of approximately 150-300 mm³. Mice then received fortnightly i.v. injections of AFF4-WT-VC-MMAE or vehicle, respectively, and tumor growth was monitored at regular intervals over up to 60 days.

FIG. 20 shows the bystander cytotoxic activity of AFF4-WT-VC-MMAE in vitro. Serial dilutions of AFF4-WT-VC-MMAE were pre-incubated for 4 days on either L1CAM high-expressing JIMT-1 cells, L1CAM low-expressing MDA-MB-468 cells, or in the absence of cells, respectively. Supernatants from these pre-incubations were transferred four-fold diluted to MDA-MB-468 cells, followed by an incubation for 6 days. Cell viability was then assessed by ATP quantification, and % cell viability was calculated, using untreated cells and cells incubated in the presence of 10 UM Doxorubicin, respectively, as 100% and 0% viability reference values. Shown are mean values from duplicates and standard deviations. The indicated AFF4-WT-VC-MMAE concentrations refer to final concentrations on MDA-MB-468 cells incubated with the supernatants. Mean values were fitted to 4 parameter logistic curves using GraphPad Prism software.

### EXAMPLES

### Example 1

### Generation of Monoclonal Antibody OV549.20 and Recombinant Expression as Mouse IgG2a and Human IgG1 Chimeric Antibody

The mouse antibody OV549.20 was generated using hybridoma technology using SKOV3ip human ovarian carcinoma cells as immunogen.

The DNA sequences encoding the heavy and light chain variable domains of OV549.20 were cloned in frame into expression vectors encoding the heavy chain and light chain constant domains of either mouse IgG2a or human IgG1, respectively. The resulting protein sequences of the entire heavy and light chains of the antibody constructs are shown in SEQ ID NO: 35 and SEQ ID NO: 36 for the murine antibody). The chimeric antibody has the same variable regions, but contains human IgG1 constant domains. The plasmids encoding the heavy and light chain of the antibody, respectively, were purified under low-endotoxin conditions and were transiently co-transfected into Chinese Hamster Ovary (CHO) K1 cells. Cells were grown in a chemically defined animal-component-free medium and supernatants containing recombinant antibodies were harvested by centrifugation and subsequent filtration through a 0.2 μm filter. Antibodies were purified from supernatants by affinity chromatography using protein A columns (MabSelect SuRe, GE Healthcare) and stored in phosphate-buffered saline (PBS) containing 100 mM arginine.

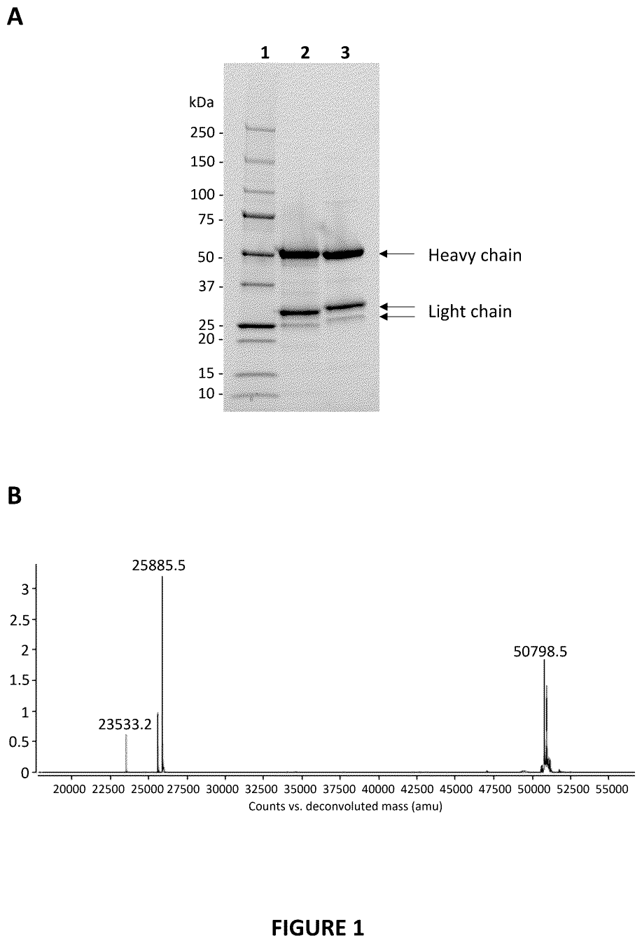

Reducing SDS-PAGE analysis of the purified OV549.20 mouse IgG2a and chimeric OV549.20 human IgG1 antibody (FIG. 1A) revealed for both antibodies a band at approximately 50 kDa, a faint band at approximately 25 kDa, as well as an additional strong band at approximately 30 kDa. Mass spectrometry analysis of chimeric OV549.20 human IgG1 (FIG. 1B) confirmed the presence of the predicted molecular masses of the heavy chain (50798.5 Da) and the light chain (23533.2 Da), and detected an additional mass of 25885.5 Da, which strongly exceeded in its amount the one of the predicted light chain. The difference in the molecular weights of 2352.3 Da between the predicted light chain mass and the additional mass is consistent with an N-glycosylation reaction occurring within the light chain sequence, indicating that the additional mass corresponds to a glycosylated variant of the light chain. In fact, sequence analysis indicated the presence of a canonical N-glycosylation motif (Asn-Ile-Thr) within the variable region of the OV549.20 light chain (SEQ ID NO: 36).

### Example 2

### In Vitro Pharmacology of OV549.20 Mouse IgG2a and Chimeric OV549.20 Human IgG1

### Expression of Extracellular Domains of Human and Cynomolgus Monkey L1CAM

In order to determine the target binding affinity of OV549.20 mouse IgG2a and chimeric OV549.20 human IgG1, recombinant versions of the extracellular portions of human L1CAM and cynomolgus monkey L1CAM were first cloned, expressed and purified. To this end, the DNA sequences encoding amino acids 20 to 1120 of human L1CAM (Uniprot accession number P32004) and the respective sequences encoding amino acids 20 to 1120 of cynomolgus monkey L1CAM (NCBI accession number XP_005594994), were synthesized and cloned into eukaryotic expression vectors. The respective recombinant expression products were designed to include N-terminal leader peptides for secretion into the cell supernatant and C-terminal hexa-histidine tags for subsequent affinity purification. Plasmid DNA was purified under low endotoxin conditions and used for the transient transfection of human embryonic kidney (HEK) cells. Transfected HEK cells were grown in 1 L expression cultures and supernatants were harvested by centrifugation. Recombinant proteins were purified from supernatants by protein A affinity chromatography and stored in PBS pH 7.4 until further analysis.

### Determination of Kinetic Binding Constants of OV549.20 Mouse IgG2a and Chimeric OV549.20 Human IgG1 Towards Human and Cynomolgus Monkey L1CAM

Monovalent kinetic binding constants of OV549.20 mouse IgG2a and chimeric OV549.20 human IgG1 towards human and cynomolgus monkey L1CAM were determined by surface plasmon resonance using a Biacore T200 instrument. Antibodies were captured via their fragment crystallizable (Fc) regions on a CM5 Protein A Chip (GE Healthcare) and soluble His-tagged human or cynomolgus monkey L1CAM, respectively, were injected as analytes in 5 different concentrations ranging from 1.25 nM to 20 nM, using 10 mM 4-(2-hydroxyethyl)-1-piperazineethanesulfonic acid 150 mM (HEPES), NaCl, 3 mM ethylenediaminetetraacetic acid (EDTA), 0.05% Tween 20 as running buffer. Detected resonance units (sensorgrams) were fitted to a 1:1 Langmuir binding model and association (kₐ) and dissociation (k_{d}) rate constants as well as the affinity constants (K_{D}) were calculated. Table 7 shows that OV549.20 mouse IgG2a and chimeric OV549.20 human IgG1 bound to both human and cynomolgus monkey with similar affinities:

TABLE 7
Binding affinities of OV549.20 mouse IgG2a and chimeric
OV549.20 human IgG1 to human and cynomolgus monkey L1CAM

| | Human L1CAM | | | Cynomolgus monkey L1CAM | | |
|---|---|---|---|---|---|---|
| | kₐ | | | kₐ | | |
| | (M⁻¹ × | k_{d} | K_{D} | (M⁻¹ × | k_{d} | K_{D} |
| Antibody | s⁻¹) | (s⁻¹) | (nM) | s⁻¹) | (s⁻¹) | (nM) |
| OV549.20 | 8.99 × | 8.03 × | 8.9 | 9.32 × | 1.07 × | 11.5 |
| mouse | 10⁴ | 10⁻⁴ | | 10⁴ | 10⁻³ | |
| IgG2a | | | | | | |
| chimeric | 8.18 × | 6.39 × | 7.8 | 7.50 × | 1.02 × | 13.5 |
| OV549.20 | 10⁴ | 10⁻⁴ | | 10⁴ | 10⁻³ | |
| human | | | | | | |
| IgG1 | | | | | | |

### Binding of Chimeric OV549.20 Human IgG1 to Human Cancer Cell Lines Expressing L1CAM

The breast cancer cell line JIMT-1 and the pancreatic cancer cell line Panc-1 were cultured in Dulbecco's Modified Eagle Medium (DMEM) containing 10% fetal calf serum (FCS). The ovarian cancer cell line SKOV3 was cultured in McCoy's 5a medium containing 10% FCS. Cells were detached from the tissue culture plates by a 10 min incubation with Accutase® cell detachment solution (5 mL per T75 cell culture flask), washed once with PBS and incubated for 1 h at 4° C. with 2 μg/ml chimeric OV549.20 human IgG1 or 2 μg/ml of an unrelated chimeric human IgG1 isotype control antibody. Alexa Fluor 488-labeled AffiniPure Goat Anti-Human IgG antibody (Jackson Immuno Research) was then added at a concentration of 5 μg/ml and incubation was continued for 1 h at 4° C. Finally, cells were incubated for 15 min at 37° C. with 10 μg/ml Hoechst-33342 solution (ChemoMetec) and analysed on an image-based cytometer (Nucleocounter NC-3000, ChemoMetec) using an excitation wavelength of 475 nm and an emission filter at 560 nm±35 nm. A total of 4,000 to 11,000 cells were analysed per condition and the detected fluorescence intensities were plotted as histograms on semi-logarithmic scales.

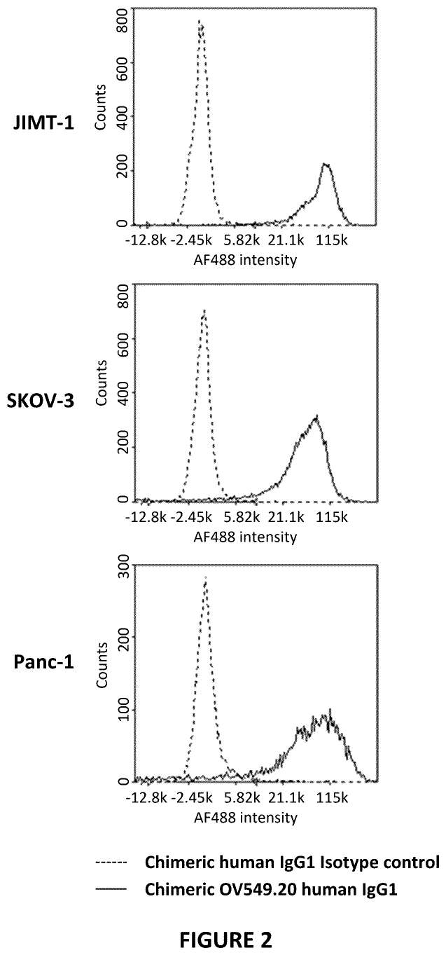

FIG. 2 shows that incubation of JIMT-1, Panc-1 and SKOV3 cells with chimeric OV549.20 human IgG1 resulted in a strong shift in the fluorescence signal compared to the isotype control antibody, demonstrating specific binding of chimeric OV549.20 human IgG1 to L1CAM on the surface of these human cancer cell lines.

### Determination of Cross-Reactivity to L1CAM Homologues NrCAM, CHL1, Neurofascin

The ability of OV549.20 mouse IgG2a to bind to other members of the L1 family of adhesion molecules, namely close homolog of L1 (CHL1), Neurofascin, and Neuronal cell adhesion molecule (NrCAM) was tested in a direct ELISA. Recombinant human L1CAM-human Fc and human NrCAM-human Fc fusion proteins, as well as recombinant hexahistidine-tagged versions of human CHL1 and Neurofascin, respectively, were added to ELISA plates at a concentration of 1 μg/mL in 50 mM carbonate buffer pH 9.6 (100 μL per well). Plates were incubated overnight at 4° C., washed 5 times with PBS/0.05% Tween, and blocked by the addition of 200 μL per well PBS/0.05% Tween/1% Bovine serum albumin (BSA). OV549.20 mouse IgG2a was then serially diluted 1:3 in PBS/0.05% Tween/1% BSA, starting from a maximal concentration of 10 μg/mL. As positive controls the same dilution series were prepared for commercial mouse antibodies directed against L1CAM (clone UJ127.11, NovusBio), human NrCAM (clone 308000, Creative Diagnostics), human CHL1 (clone 6E6E4, Sino Biological) and human Neurofascin (clone 727030, R&D systems). Plates were washed 5 times with PBS/0.05% Tween, and 100 μL per well of the antibody dilution series were added to the plates. After an incubation for 1 hour at room temperature, plates were washed again and 100 μL per well of a horse-radish peroxidase conjugated polyclonal goat anti-mouse antibody (diluted 1:1000 in PBS/0.05% Tween/1% BSA) was added. After an additional incubation for 1 h at room temperature, plates were washed again and 100 μL per well TMB (3,3′,5,5′-tetramethylbenzidine) substrate was added for detection. After an incubation of 8 minutes at room temperature, reactions were stopped by the addition of 50 μL per well 0.16 M H₂SO₄, and absorption at 450 nm was determined in a microplate reader.

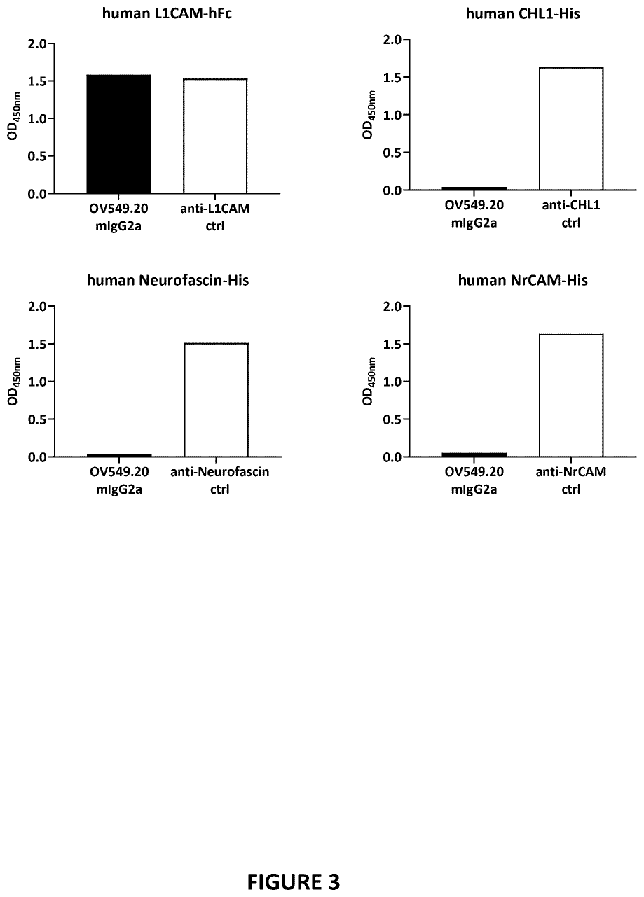

FIG. 3 shows the OD values obtained in the different ELISA assays at an antibody concentration of 1 μg/mL. The control antibodies specific for human L1CAM, CHL1, NrCAM, and Neurofascin showed strong binding to their respective target antigens, demonstrating successful immobilization of all antigens on the ELISA plates and correct functionality of the detection system. OV549.20 mouse IgG2a showed strong binding to plate-coated human L1CAM, but did not show any reactivity with plate-coated human CHL1, NrCAM or Neurofascin.

### Induction of Antibody-Dependent Cellular Cytotoxicity (ADCC) by Chimeric OV549.20 Human IgG1

Panc-1 human pancreatic cancer cells expressing L1CAM and Her2 on their surface were labeled with Cr⁵¹ and pre-incubated for 20 minutes at room temperature with 8 different concentrations of chimeric OV549.20 human IgG1 antibody. Herceptin was used as a positive control and a chimeric human IgG1 version of the anti-L1CAM antibody L9.3 (WO 2008/151819) binding to the first Ig domain of L1CAM was tested in parallel. Human CD16 (158V alloform) transgenic T cells were then added as effector cells at an effector to target ratio of 20:1 and incubation was continued for 4 hours at 37° C. in a humidified atmosphere with 5% CO₂. After incubation, cells were centrifuged and 25 μl of supernatant were transferred to Lumaplates (Perkin Elmer). Target cell lysis was measured by supernatant gamma counting and expressed in corrected counts per minute (CCPM) after detector normalization. Spontaneous lysis was determined by gamma counting of supernatants of Panc-1 cells incubated in culture medium in the absence of effector cells and antibody. Maximum lysis was determined by gamma counting of supernatants from Panc-1 cells lysed by the addition of 0.75% Triton X-100. The percentage of specific lysis was calculated according to the following formula:

$$\% Specific lysis = ( \frac{{CCPM}_{A b} - {CCPM}_{S L}}{{CCPM}_{Max} - {CCPM}_{S L}} ) \times 100$$

where CCPM_{Ab} are the counts of the wells containing Panc-1 cells, effector cells and antibody, CCPM_{SL} are the counts of the wells containing only Panc-1 cells in the absence of effector cells and antibody, and CCPMₘₐₓ are the counts of the wells of Panc-1 cells lysed with Triton X-100. Maximal lysis (Eₘₐₓ) as well as half maximal lysis (EC₅₀) values were calculated after fitting specific lysis values to a 4-parameter logistic curve.

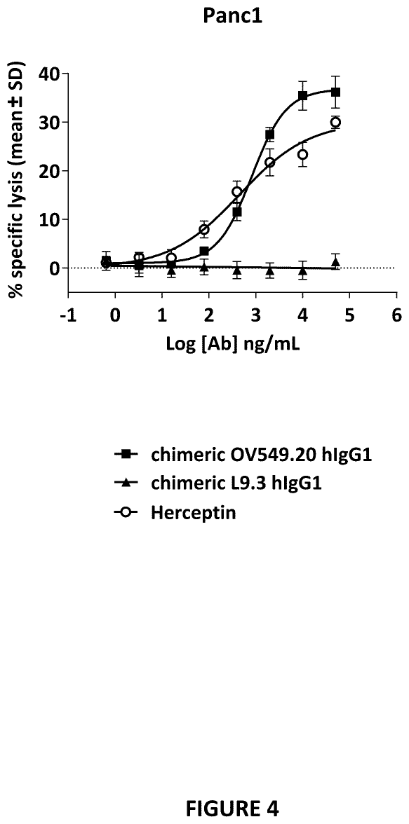

FIG. 4 shows that chimeric OV549.20 human IgG1 induced robust ADCC on Panc-1 target cells. The antibody induced a dose dependent increase in the specific lysis of Panc-1 cells, with an Eₘₐₓ of 36.8% and an EC₅₀ of 0.82 μg/mL. The positive control antibody Herceptin mediated lysis of Panc-1 cells with an Eₘₐₓ of 30.1% and an EC₅₀ of 0.45 μg/mL. The chimeric human IgG1 version of the previously described antibody L9.3 binding to the first Ig domain of L1CAM did not induce any ADCC on Panc-1 target cells.

### Inhibition of Cancer Cell Proliferation by OV549.20 Mouse IgG2a

HCT116 colorectal cancer cells (in DMEM: Ham's F12 supplemented with 0.5% FBS), Panc-1 pancreatic cancer cells (in DMEM supplemented with 4.5 g/L glucose, glutamine, and 0.5% FBS) and SKOV3 ovarian cancer cells (in McCoys 5a supplemented with 1.5. mM glutamine, 2.2. g/L sodium bicarbonate, and 0.5% FBS) were seeded on 384 well plates at 10-15% confluency either with vehicle alone or in the presence of 50 μg/mL OV549.20 mouse IgG2a antibody, 50 μg/mL of a mouse IgG2a isotype control antibody, or a mouse IgG2a version of the previously described antibody OV52.24 (WO2016050702). Proliferation of cancer cells was monitored at regular intervals over 96 hours by microscopic confluency detection using an Incucyte device.

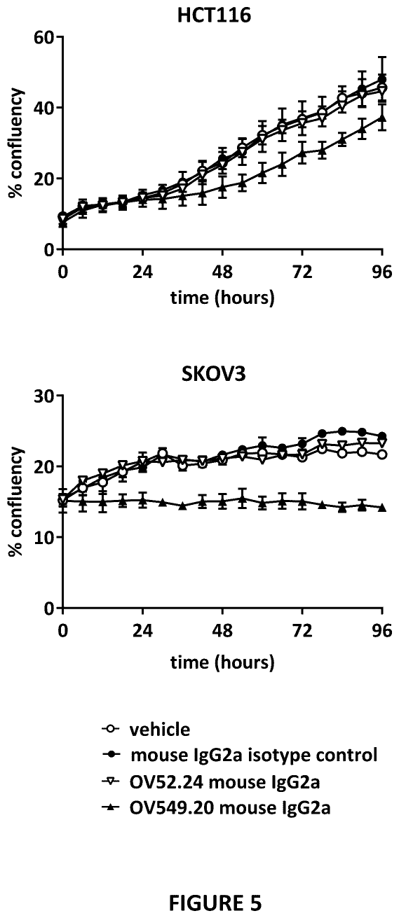

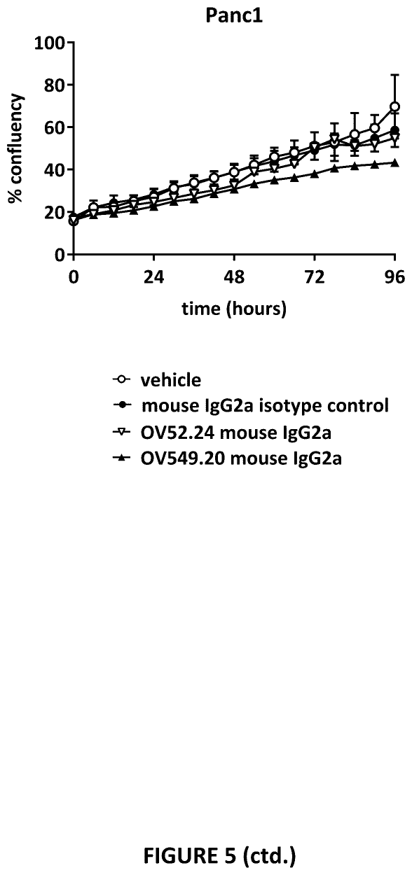

FIG. 5 shows that the mouse IgG2a isotype control antibody and the OV52.24 mouse IgG2a antibody had no influence on the proliferation of either cancer cell line, while addition of OV549.20 mouse IgG2a antibody led to a reduction in the proliferation of all three cancer cell lines. By 96 hours the addition of OV549.20 mouse IgG2a had caused a reduction of confluency from 45.7% to 37.3% for HCT116 cells, from 21.7% to 14.2% for SKOV3 cells, and from 69.7% to 43.3% for Panc-1 cells, when compared to cells treated with vehicle only.

### Inhibition of Cancer Cell Migration by Chimeric OV549.20 Human IgG1

HCT116 colorectal cancer cells were cultured in DMEM supplemented with 10% FCS, harvested with Accutase® cell detachment solution (Capricorn) and seeded at a density of 4×10⁴ cells/100 μL into stopper-containing wells of a fibronectin precoated ORIS-96 well plate (AMS-bio). Twenty-four hours later, stopper inserts were removed, and medium was exchanged with 120 μL fresh growth medium. Subsequently, 30 μl of a 5× concentrated serial dilution series of chimeric OV549.20 human IgG1 or chimeric human IgG1 isotype control, respectively, was added to the cells. Final concentrations of the antibodies in the assay plate ranged from 4×10⁻⁵ M to 1.2×10⁻⁸ M. As positive control for unrestricted migration, cells were incubated in the absence of any antibody. As negative control, migration was restricted by maintaining stoppers in eight wells per plate. After 48 hours, medium was substituted with 75 μL of phenol red-free DMEM supplemented with 2 μg/ml Calcein-AM (Life Technologies). Cells were incubated for 15 min at 37° C. and fluorescent cells in the insert-defined area were detected by a fluorescence microplate reader (Fluostar, BMG) using fluorescein isothiocyanate (FITC)-settings (Excitation: 485 nm/Emission: 520 nm). The median value of the positive control (stopper removed, no addition of antibody) was set to 100% (high control) and the median value of the negative control (stopper not removed) was set to 0% (low control). Raw data were converted into % cell migration relative to the controls and were fitted to a 4-parameter logistic curve using a bottom constrain of 0 and a top constrain of 100 with a variable slope.

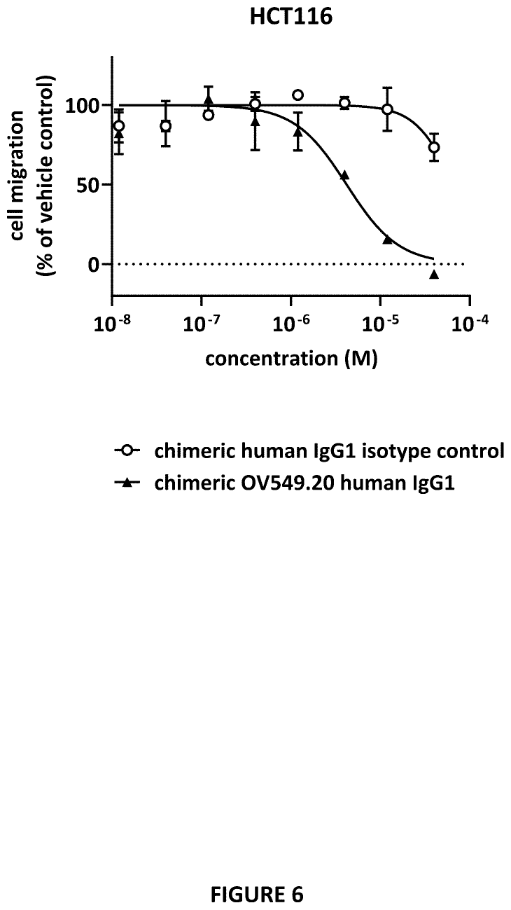

FIG. 6 shows that addition of the chimeric human IgG1 isotype control antibody had negligible effects on the migration of HCT116 cells, while addition of the chimeric OV549.20 human IgG1 antibody resulted in a specific, dose-dependent inhibition of migration. The half maximal inhibitory concentration (IC₅₀) for chimeric OV549.20 human IgG1 was calculated to 4.2×10⁻⁶ M.

### Example 3

### In Vivo Pharmacology of OV549.20 Mouse IgG2a

### Inhibition of Primary Tumor Growth in SKOV3 Ovarian Cancer Xenograft Model

The ability of OV549.20 mouse IgG2a to inhibit primary tumor growth was investigated in a SKOV3 ovarian cancer xenograft model. SKOV3 ovarian cancer cells were cultured in DMEM containing 10% FCS and 1 mM glutamine. At 60-70% confluency, cells were harvested with 0.05% trypsin and 0.02% EDTA in PBS and injected intraperitoneally into 30 female NMRI: nu/nu mice (5×10⁶ cells per mouse). After 5 days, mice were divided into three groups of 10 animals each, which received intraperitoneal injections of OV549.20 mouse IgG2a antibody, L9.3 mouse IgG2a (WO 2008/151819), or a mouse IgG2a isotype control antibody, respectively, each at 10 mg/kg. Treatment was repeated 3 times per week over a period of 6 weeks, after which animals were sacrificed and SKOV3 tumor weights as well as ascites volumes in peritoneal cavities were determined.

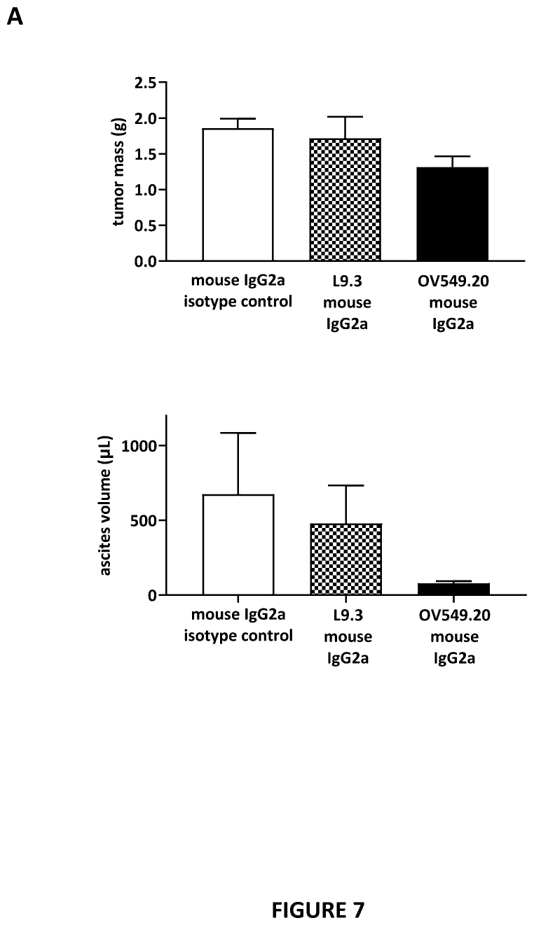

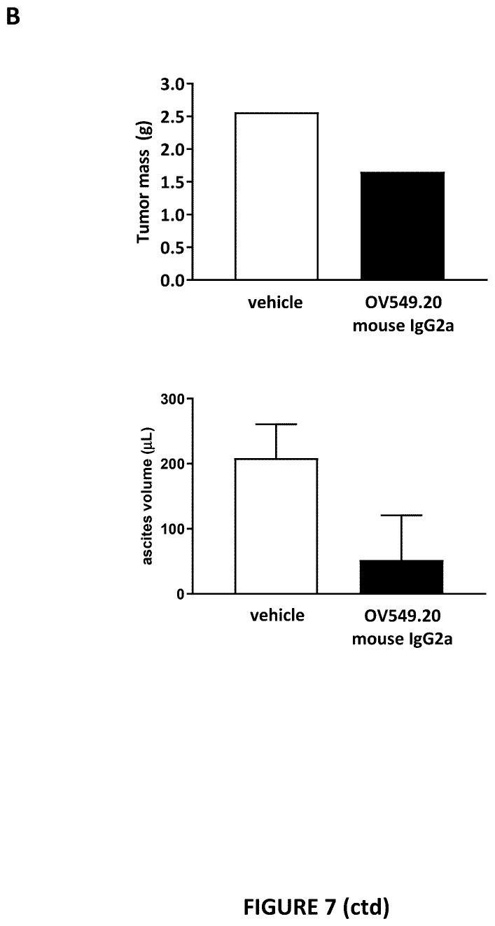

FIG. 7A shows that treatment with the OV549.20 mouse IgG2a antibody caused a 29% reduction in the mean tumor mass compared to the isotype-treated control group. Furthermore, the development of ascites fluid was reduced by 88% in OV549.20 mouse IgG2a treated animals compared to isotype treated control animals. In contrast, treatment with L9.3 mouse IgG2a induced no reduction of tumor mass and only a minor reduction of ascites fluid.

In a second experiment 20 female NMRI: nu/nu mice were injected intraperitoneally with SKOV3 cells (5·10⁶ cells per mouse). Five days after injection, mice were divided into two groups of 10 mice each and were injected intraperitoneally with OV549.20 mouse IgG2a (10 mg/kg) or vehicle, respectively. Treatment was repeated three times per week for a total of 6 weeks, followed by analyses of tumor weights and ascites volumes in peritoneal cavities.

FIG. 7B shows that treatment with OV549.20 mouse IgG2a caused a reduction of 35% in the median tumor load and a reduction of 75% in the ascites volume when compared to the vehicle treated group.

### Inhibition of Metastasis Formation in MDA-MB-231 Breast Cancer Xenograft Model

The ability of OV549.20 mouse IgG2a to inhibit metastasis formation was tested in a mouse xenograft model of human MDA-MB-231-luc2 metastatic breast cancer cells. In this model, intravenously administered MDA-MB-231-luc2 cells metastasize to the lungs of mice, where they can be detected by whole body luminescence after the administration of a luminescent substrate.

Groups of female athymic Nude-Foxn1nu mice (n=15 per group) were injected intravenously three times per week for five weeks with either OV549.20 mouse IgG2a antibody (10 mg/kg) or with vehicle control, respectively. Three days after the first administration, all animals were injected intravenously with 5×10⁵ MDA-MB-231-luc2 cells. Clinical signs and body weights were recorded daily, and in vivo whole-body luminescence imaging was performed for all animals at 7, 14, 21 and 30 days after cell injection. To this end animals were administered D-luciferin (150 mg/kg) via intraperitoneal injection and were imaged 10 min later, while under isoflurane anesthesia. Images were captured and luminescence signals (total flux in p/s) were measured in the thoracic region.

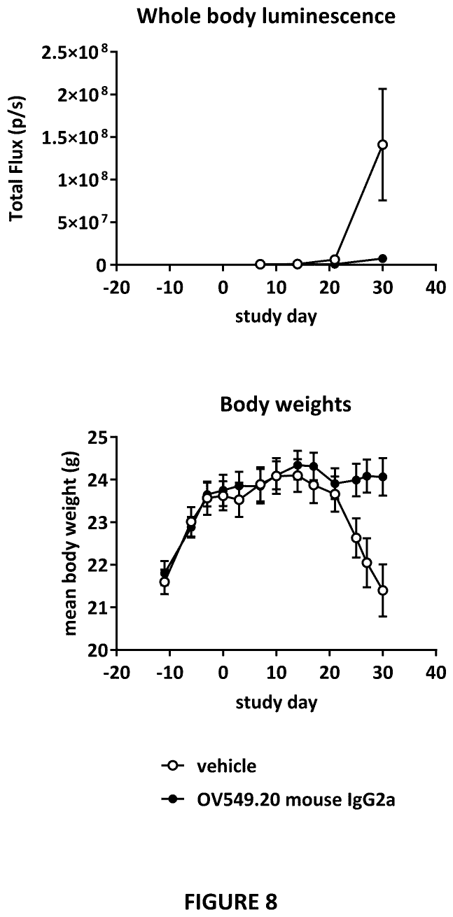

FIG. 8 shows that treatment with OV549.20 mouse IgG2a strongly inhibited metastasis formation, as determined by whole body luminescence signals. On day 30 total flux signals in OV549.20 mouse IgG2a treated mice were reduced by 94.8% compared to vehicle control. While vehicle control animals showed a strong and sustained decrease in body weight after day 20, OV549.20 mouse IgG2a treated mice did not show any detectable body weight loss over the course of the experiment. Treatment with OV549.20 mouse IgG2a thus potently inhibited metastasis formation and improved clinical signs of disease.

### Example 4

### Humanization of OV549.20

### In Silico Grafting of CDRs of OV549.20 on Human Framework

Based on computational modeling of OV549.20 Fv, the human VH sequence IGHV4 30 4 01 was identified as the most appropriate acceptor framework for the CDRs of the heavy chain of OV549.20. The VH CDRs of OV549.20 (SEQ ID NOs: 1, 2, and 3; cf. Table 1) were grafted in silico on IGHV4 30 4 01, and at the same time certain positions in the human framework of IGHV4 30 4 01 were back-mutated to the corresponding mouse residues, whenever this was deemed appropriate to avoid new contacts or maintain existing contacts within the modeled OV549.20 Fv. In this way, four humanized variants of the heavy chain variable regions of OV549.20 were generated, including varying numbers of back-mutated framework residues (H1-H4, SEQ ID NOs 23, 24, 25, 26).

In parallel, the human VL sequence IGKV1 39 01 was identified as the most suitable human acceptor framework for the CDRs of the light chain of OV549.20. The VL CDRs of OV549.20 (SEQ ID NOs: 4, 5, and 6; cf. Table 2) were grafted in silico on

IGKV1 39 01, and at the same time a series of back-mutations of human residues to the corresponding mouse residues were introduced into the human framework, whenever this was deemed necessary to maintain structural and functional integrity. In addition, the putative N-glycosylation site (Asn-Ile-Thr), likely leading to glycosylation of the light chain upon expression in CHO cells, detectable as a mass shift in SDS-PAGE and MS (see FIG. 1), was mutated to a sequence unable to support such a glycosylation reaction (Thr-Ile-Thr). Four different humanized variants of the OV549.20 light chain variable domains were generated, each harboring the mutated N-glycosylation site and varying numbers of back-mutated framework residues. Of these, humanized light chain variable domains L1 and L2 were further used (L1: SEQ ID NO: 20, L2: SEQ ID NO: 22)

### Expression and Purification of Humanized Variants of OV549.20

The DNA sequences encoding the four different humanized heavy chain variable domains (H1-H4) were each fused in frame to a DNA sequence encoding the human IgG1 constant domains and used to generate expression constructs for the full length humanized IgG1 heavy chains. Similarly, the DNA sequences encoding two of the light chain variable domains (L1 and L2) were each fused in frame to a sequence encoding a light chain constant domain and used to generate expression constructs for two full length humanized kappa light chains. Each of the four heavy chain expression plasmids was transiently co-transfected with either of the two light chain expression plasmids into CHO cells, resulting in the expression of 8 different full length humanized IgG1 antibody variants (H1L1 to H4L2). Cells were grown in a chemically defined animal-component-free medium and supernatants containing recombinant antibodies were harvested by centrifugation and subsequent filtration through a 0.2 μm filter. Antibodies were purified from supernatants by affinity chromatography using protein A columns (MabSelect SuRe, GE Healthcare) and stored in PBS containing 100 mM arginine.

### Biophysical Characterization of Humanized Variants of OV549.20

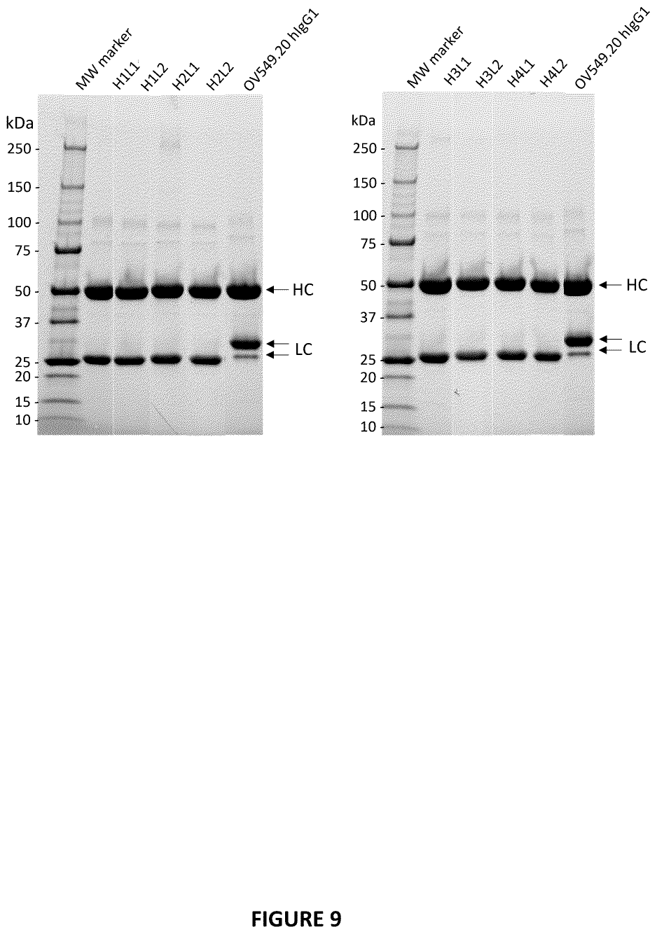

All eight humanized antibodies were analysed by reducing SDS-PAGE and compared to the parental chimeric OV549.20 human IgG1. FIG. 9 shows that all humanized antibodies showed a single band at approximately 50 kDa, corresponding to the heavy chain, and a single band at approximately 25 kDa corresponding to the light chain. In contrast to the parental chimeric OV549.20 human IgG1 antibody, the additional prominent band at 30 kDa, likely representing a glycosylated variant of the light chain, was not detected in any of the humanized antibodies.

Conformational stability of the humanized antibodies was analysed by differential scanning fluorimetry using Sypro Orange. As shown in Table 8, all humanized antibody variants showed a first melting point (Tₘ1) at approximately 70° C., which was similar to the parental chimeric OV549.20 human IgG1 antibody. The second melting point (Tₘ2) was dependent on the respective heavy chains of the antibody variants and was above 83.8° C. in all cases, indicating high conformational stability of all humanized antibody variants.

TABLE 8
First and second melting point of humanized
variants of OV549.20 in comparison to parental
chimeric OV549.20 human IgG1 and Rituximab

Melting point (° C.)

| Antibody | Tₘ1 | Tₘ2 |
|---|---|---|
| H1L1 | 70.1 | 86.4 |
| H1L2 | 70.1 | 86.6 |
| H2L1 | 70.0 | 90.0 |
| H2L2 | 70.0 | 90.2 |
| H3L1 | 70.2 | 88.8 |
| H3L2 | 70.0 | 88.7 |
| H4L1 | 70.0 | 83.9 |
| H4L2 | 70.1 | 83.8 |
| Chimeric OV549.20 human IgG1 | 70.1 | 86.7 |
| Rituximab | 69.7 | 75.3 |

Monovalent kinetic binding constants of the eight humanized antibody variants towards human L1CAM were determined by surface plasmon resonance using a Biacore T200 instrument. Antibodies were captured via their Fc regions on a C1 Protein A Chip (GE Healthcare), and soluble His-tagged human L1CAM was injected as analyte in 3 different concentrations (10, 20 and 40 nM), using 10 mM HEPES/150 mM NaCl/3 mM EDTA/0.05% Tween 20 as running buffer. Detected resonance units (sensorgrams) were fitted to a 1:1 Langmuir binding model and association (kₐ) and dissociation (k_{d}) rate constants as well as the affinity constants (K_{D}) were calculated. Table 9 shows that all humanized antibody variants bound to human L1CAM with high affinity and displayed similar kinetic binding constants.

TABLE 9
Monovalent kinetic binding constants
of humanized variants of OV549.20

Kinetic binding constants (human L1CAM)

| Antibody | Kₐ (M⁻¹ × s⁻¹) | K_{d} (s⁻¹) | K_{D} (nM) |
|---|---|---|---|
| H1L1 | 1.77 × 10⁵ | 7.90 × 10⁻⁴ | 4.5 |
| H1L2 | 1.44 × 10⁵ | 8.72 × 10⁻⁴ | 6.1 |
| H2L1 | 1.70 × 10⁵ | 7.51 × 10⁻⁴ | 4.4 |
| H2L2 | 1.44 × 10⁵ | 8.67 × 10⁻⁴ | 6.0 |
| H3L1 | 1.68 × 10⁵ | 8.58 × 10⁻⁴ | 5.1 |
| H3L2 | 1.31 × 10⁵ | 8.87 × 10⁻⁴ | 6.8 |
| H4L1 | 1.79 × 10⁵ | 8.47 × 10⁻⁴ | 4.7 |
| H4L2 | 1.37 × 10⁵ | 8.59 × 10⁻⁴ | 6.3 |

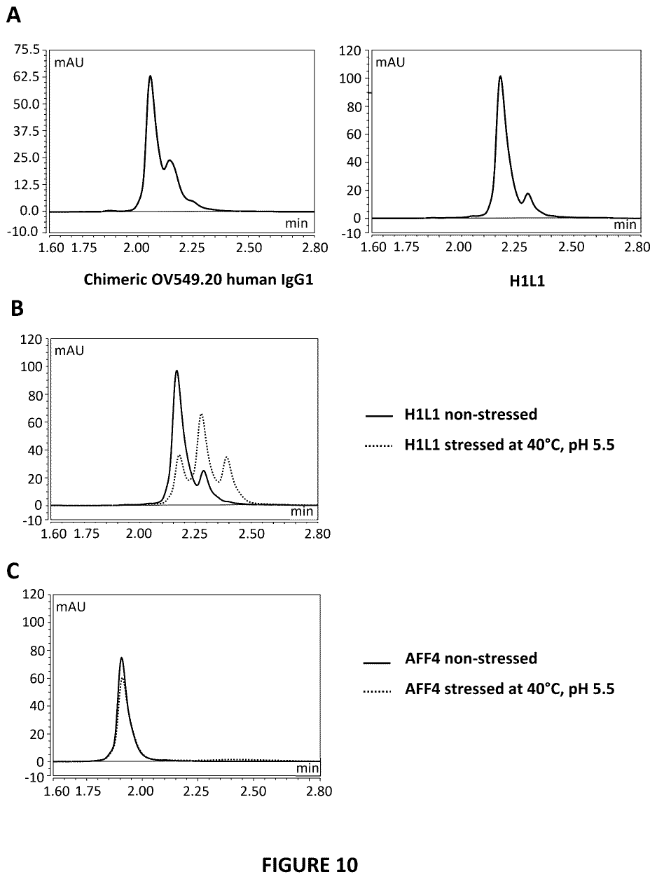

All humanized antibody variants were analysed by hydrophobic interaction chromatography on a Tosoh TSKgel Butyl-NPR column, and compared to the parental chimeric OV549.20 human IgG1. The exemplary chromatogram in FIG. 10A shows that the parental chimeric antibody OV549.20 human IgG1 displayed a major peak and an additional minor peak/shoulder after the main peak, indicating the presence of posttranslational variants within the preparation. The humanized variant H1L1 displayed the same peak pattern, however the additional peak was surprisingly markedly reduced compared to the parental chimeric antibody, indicating a reduction in the presence of posttranslational variants. Peak Integration of the HIC chromatograms of all humanized antibody variants (Table 10) showed that all variants had a similarly reduced area of the minor peak, indicating that grafting on all human framework variants reduced the formation of posttranslational variants in comparison to the parental chimeric antibody.

TABLE 10
Relative peak areas of humanized variants of OV549.20
in hydrophobic interaction chromatography

Relative peak area in HIC (%)

| Antibody | Peak 1 | Peak 2 |
|---|---|---|
| Chimeric OV549.20 human | 65.3 | 34.2 |
| IgG1 | | |
| H1L1 | 82.0 | 15.8 |
| H1L2 | 81.8 | 16.4 |
| H2L1 | 85.2 | 13.1 |
| H2L2 | 83.8 | 14.8 |
| H3L1 | 82.3 | 16.0 |
| H3L2 | 81.9 | 16.5 |
| H4L1 | 80.8 | 17.3 |
| H4L2 | 79.1 | 19.1 |

The nature of the posttranslational variant detected in hydrophobic interaction chromatography was investigated in detail by exposing the humanized antibody variant H1L1 to high temperature and low pH stress. As shown in FIG. 10B, incubation of H1L1 for twelve days at 40° C. in 20 mM sodium citrate pH 5.5 markedly increased the magnitude of the second minor peak and led to the appearance of a prominent third peak. This indicated that a posttranslational modification reaction preferentially occurring at low pH values, such as an isomerization reaction or similar was at the origin of the heterogeneity detected in the hydrophobic interaction chromatography. Indeed, proteolytic digestion of the non-stressed and the pH-stressed H1L1 with AccuMAP™ Low pH Protein Digestion Kit, followed by reverse phase liquid chromatography mass spectrometry and MS-MS analysis of the peptide corresponding to amino acids 1 to 39 of the heavy chain of H1L1 revealed that low pH stress leads to the loss of a mass of 18 Da at a position located between amino acid 31 and 34. Such a modification is consistent with an isomerization reaction at the aspartate residue at position 32, which leads to the loss of a water molecule (−18 Da) and the formation of a stable succinimide intermediate.

### Example 5

### Affinity Improvement and Optimization of Biophysical Properties and Effector Functions of Humanized Antibodies

The humanized antibody H1L1 was selected as a basis for the further optimization of target affinity, biophysical properties and effector functions. Three different scFv phage display libraries were constructed, where selected amino acid positions in the CDRs of the heavy and light chain of H1L1 were randomized, using a degenerate oligonucleotide-directed PCR mutagenesis approach. After four rounds of panning on recombinant human L1CAM under various types of selective pressure, a total of 470 clones was randomly selected and subjected to monoclonal phage ELISA on human L1CAM coated plates, using the parental phage as control. Sixty-two clones with strongly enhanced binding signals in ELISA were selected and subjected to DNA sequencing. Twenty unique sequences were identified, of which 13 were expressed in soluble scFv format and subjected again to ELISA on human L1CAM coated plates. Increased binding compared to the parental scFv was detected for 11 out of the 13 tested scFvs. Based on the sequences of the high-binding clones eight different modified heavy chain variable domains (SEQ ID NOs: 27-34) and one modified light chain variable domain (SEQ ID NO: 21) were then designed in addition to light chain variable domain SEQ ID NO: 20. Heavy chain and light chain variants were combined to yield 10 different heavy and light chain combinations, and the respective expression plasmids for the corresponding full length human IgG1 antibodies were constructed. In order to improve the Fc-mediated effector functions of the antibodies, namely their ability to induce antibody-dependent cellular cytotoxicity (ADCC), four mutations (G236A/S239D/A330L/I332E, EU numbering) were introduced into the CH2 domain of the heavy chain. These mutations have been described to selectively enhance the affinity of the Fc domain to activating Fc gamma receptors [18]. The resulting full length antibodies AFF1 to AFF10 were expressed and purified and subjected to detailed biophysical and in vitro pharmacological analyses as described in Example 4.

The monovalent kinetic binding constants towards human L1CAM were determined by surface plasmon resonance using a Biacore T200 instrument as described in Example 4, except that soluble His-tagged human L1CAM was injected in 4 different concentrations (5, 10, 20 and 40 nM). Table 11 shows that all mutated variants displayed improved binding to human L1CAM compared to the parental antibody H1L1, with affinity constants (K_{D}) in the subnanomolar range in all cases.

TABLE 11
Binding affinities of further mutated
humanized antibodies (AFF1-AFF10)

| | | | Kinetic binding constants (human | | |
|---|---|---|---|---|---|
| | | VL | L1CAM) | | |
| Anti- | VH | muta- | | | K_{D} |
| body | mutation | tion | Kₐ (M⁻¹ × s⁻¹) | K_{d} (s⁻¹) | (nM) |
| AFF1 | H61N G103S | — | 2.90 × 10⁵ | 1.04 × 10⁻⁴ | 0.36 |
| AFF2 | A34T H61N | — | 4.11 × 10⁵ | 1.26 × 10⁻⁴ | 0.31 |
| | G103S | | | | |
| AFF3 | F58Y H61N | — | 2.99 × 10⁵ | 6.52 × 10⁻⁵ | 0.22 |
| | G103S | | | | |
| AFF4 | A34T F58Y | — | 4.16 × 10⁵ | 8.45 × 10⁻⁵ | 0.20 |
| | H61N G103S | | | | |
| AFF5 | A34T F58Y | — | 3.49 × 10⁵ | 2.52 × 10⁻⁴ | 0.72 |
| | F100L | | | | |
| AFF6 | A34T H61N | — | 3.45 × 10⁵ | 3.37 × 10⁻⁴ | 0.98 |
| | F100L | | | | |
| AFF7 | H61N G103S | T56I | 2.73 × 10⁵ | 8.83 × 10⁻⁵ | 0.32 |
| AFF8 | A34T H61N | T56I | 3.83 × 10⁵ | 1.16 × 10⁻⁴ | 0.30 |
| | G103S | | | | |
| AFF9 | F58Y H61N | — | 2.58 × 10⁵ | 9.28 × 10⁻⁵ | 0.36 |
| | G103A | | | | |
| AFF10 | A34T F58Y | — | 2.55 × 10⁵ | 2.51 × 10⁻⁴ | 0.99 |

Chemical stability of antibodies AFF1 to AFF10 was analyzed by hydrophobic interaction chromatography on a Tosoh TSKgel Butyl-NPR column and compared to the parental antibody H1L1. Surprisingly, in contrast to H1L1, none of the antibody variants displayed a second peak on the hydrophobic interaction column and the after main peak areas (Table 12) were overall very low for all variants, indicating that the posttranslational variant originating from aspartate isomerization at position 32 was either not present or present in very low amounts in antibody variants AFF1 to AFF10.

Table 12 shows that exposure of the parental antibody H1L1 to high temperature and low pH stress (14 days at 40° C. in 20 mM sodium citrate buffer pH 5.5) caused a marked increase (19.5% to 76.2%) in the after main peak area in hydrophobic interaction chromatography. This increase was markedly reduced for antibody variants AFF1 to AFF10, indicating that the introduced mutations strongly inhibited the isomerization reaction at position 32. Exemplary chromatograms of the stressed and non-stressed antibody variant AFF4 are shown in FIG. 10C.

TABLE 12
Chemical stability of antibodies AFF1
to AFF10 in comparison to H1L1 (pH 5.5)

| | Non-stressed sample | | Stressed sample (pH 5.5) | |
|---|---|---|---|---|
| | Main peak | After main | Main peak | After main |
| Antibody | area (%) | peak area (%) | area (%) | peak area (%) |
| H1L1 | 79.4 | 19.5 | 23.8 | 76.2 |
| AFF1 | 97.3 | 2.6 | 82.3 | 16.6 |
| AFF2 | 97.6 | 1.5 | 88.4 | 10.4 |
| AFF3 | 97.2 | 2.5 | 84.5 | 15.0 |
| AFF4 | 97.4 | 1.7 | 87.8 | 11.1 |
| AFF5 | 98.4 | 1.0 | 83.9 | 15.3 |
| AFF6 | 98.5 | 1.1 | 84.8 | 14.6 |
| AFF7 | 96.3 | 2.5 | 84.5 | 14.4 |
| AFF8 | 97.7 | 1.7 | 88.4 | 10.6 |
| AFF9 | 96.3 | 3.1 | 76.5 | 22.5 |
| AFF10 | 97.7 | 1.9 | 85.1 | 14.2 |

### Example 6

### In Vitro Pharmacology of Humanized Optimized Antibody AFF4

### Inhibition of Cancer Cell Proliferation

HCT116, Panc-1, and SKOV3 were cultured as described in example 2 and seeded on 384 well plates at 10-15% confluency in the presence of 50 μg/mL AFF4 or 50 μg/mL of a chimeric human IgG1 isotype control antibody. Proliferation of cancer cells was monitored at regular intervals over 120 hours by microscopic confluency detection using an Incucyte device.

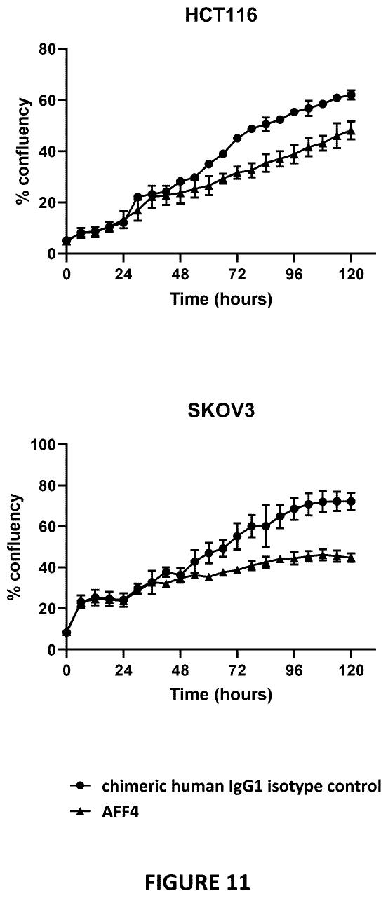

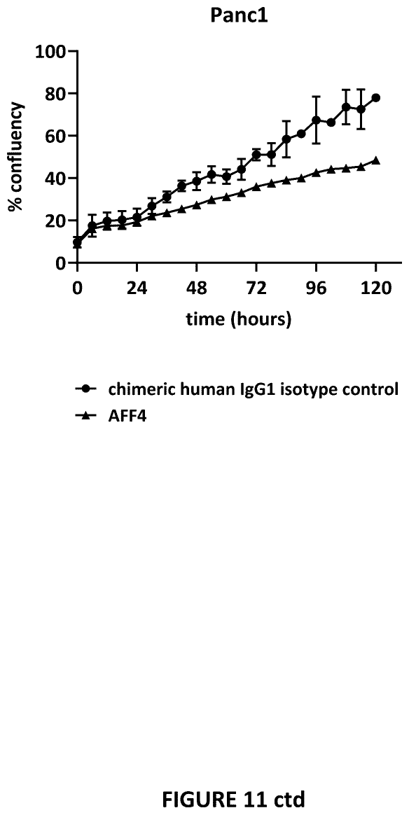

FIG. 11 shows that addition of the AFF4 antibody resulted in a reduction of proliferation of all three cell lines compared to the isotype control antibody. By 96 hours the addition of AFF4 had caused a reduction of confluency from 55.3% to 38.9% for HCT116 cells, from 68.6% to 44.4% for SKOV3 cells, and from 67.4% to 42.7% for Panc-1 cells, when compared to cells treated with isotype control antibody.

### Inhibition of Cancer Cell Migration

HCT116 colorectal cancer cells were cultured in DMEM supplemented with 10% FCS, harvested with Accutase® cell detachment solution (Capricorn) and seeded at a density of 4×10⁴ cells/100 μL into stopper-containing wells of a fibronectin precoated ORIS-96 well plate (AMS-bio). Twenty-four hours later, stopper inserts were removed, and medium was exchanged with 80 μL fresh growth medium. Subsequently, 20 μl of a 5× concentrated serial dilution series of AFF4 or chimeric human IgG1 isotype control, respectively, was added to the cells. Final concentrations of the antibodies in the assay plate ranged from 4×10⁻⁵ M to 1.2×10⁻⁸ M. As positive control for unrestricted migration, cells were incubated in the absence of any antibody. As negative control, migration was restricted by maintaining stoppers in eight wells per plate. After 48 hours, medium was substituted with 75 μL of phenol red-free DMEM supplemented with 2 μg/ml Calcein-AM (Life Technologies). Cells were incubated for 15 min at 37° C. and fluorescent cells in the insert-defined area were detected by a fluorescence microplate reader (Fluostar, BMG) using FITC-settings (Excitation: 485 nm/Emission: 520 nm). The median value of the positive control (stopper removed, no addition of antibody) was set to 100% (high control) and the median value of the negative control (stopper not removed) was set to 0% (low control). Raw data were converted into % cell migration relative to the controls and were fitted to a 4-parameter logistic curve using a bottom constrain of 0 and a top constrain of 100 with a variable slope.

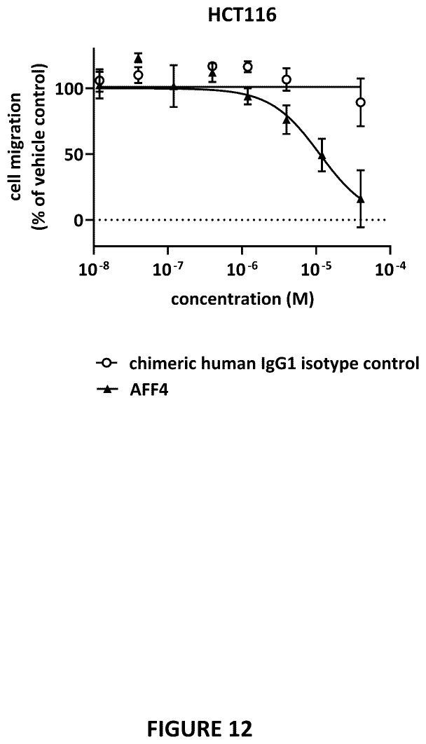

FIG. 12 shows that addition of AFF4 antibody resulted in a specific, dose-dependent inhibition of migration. The half maximal inhibitory concentration (IC₅₀) for AFF4 was calculated to about 1.1×10⁻⁵ M.

### Induction of Antibody-Dependent Cellular Cytotoxicity (ADCC) by AFF4 on SKOV3, Panc-1, HeLa, and PC03 Cancer Cell Lines

The ability of AFF4 to induce antibody-dependent cellular cytotoxicity was investigated on the L1CAM-expressing cancer cell lines SKOV3, HeLa, Panc-1 and PC03. In a first step, the level of L1CAM expression on the surface of the different cell lines was assessed by flow cytometry. Each cell line was grown in continuous culture and used 2 to 4 days after passage. A non-enzymatic cell dissociation buffer was used to detach the target cells from the surface of the culture flask prior to the assay, to limit the cleavage of surface expressed L1CAM. Cells were stained using a phycoerythrin (PE)-labeled anti-human L1CAM antibody (clone L1-OV198.5, BioLegend) or a PE-labeled mouse IgG2a (κ) (clone MOPC-173, BioLegend) as the respective isotype control antibody. Stained cells were acquired on a Quanteon flow cytometer and analysed using the NovoExpress software. Median Fluorescence Intensity (MFI) values were calculated for each cell line assessed (in triplicates) and the mean values are tabulated in Table 13. For quantification of the expression levels, the fold increase of the MFI values obtained with the anti-L1CAM antibody with respect to the isotype control antibody was calculated. All cell lines expressed L1CAM on their surface, with SKOV3 and HeLa showing the highest expression levels and Panc-1 and PC03 showing moderate and low expression levels, respectively.

TABLE 13
L1CAM expression on the surface of different cell lines as
determined by flow using a PE-labeled anti-L1CAM antibody

| | | | Fold increase |
|---|---|---|---|
| | | | in MFI |
| | MFI isotype control | MFI anti-L1CAM | (anti-L1CAM |
| Cell line | antibody | antibody | vs isotype) |
| SKOV3 | 8.68 × 10³ | 1.50 × 10⁶ | 173 |
| HeLa | 9.14 × 10³ | 1.22 × 10⁶ | 133 |
| Panc-1 | 1.31 × 10⁴ | 1.83 × 10⁵ | 14 |
| PC-03 | 9.42 × 10³ | 4.64 × 10⁴ | 5 |

For the assessment of ADCC, primary Natural Killer (NK) cells were isolated from cryopreserved peripheral blood mononuclear cells (PBMCs) of three different donors, using an NK cell isolation kit (Miltenyi Biotech). One of the donors was homozygous for the CD16 (FcγRIIIA) high affinity allele V158, one was homozygous for the CD16 low affinity allele F158, whereas the third one was heterozygous, carrying both the high and the low affinity allele (V/F158). Enriched NK cells were checked for purity (>80% CD3-CD56+ cells within the CD45+ cell population) and incubated at 36±1° C., 5±1% CO₂ in NK92 medium+IL-2 (3 ng/ml) for 21±1 hours. In parallel, pre-warmed cell medium was added to the wells of an xCelligence E-plate 96 (Agilent) (50 μL/well), and suspensions of SKOV3, HeLa, Panc-1, and PC-03 cells, respectively, were added (100 μL/well), and allowed to settle to the bottom of the wells for 1 hour at room temperature. The E-Plates were then transferred to the xCelligence Real Time Cell Analyzer (RTCA) and incubated at 36±1° C., 5±1% CO₂ for 16-24 hrs to allow cell attachment and proliferation. Impedance was measured continuously overnight to monitor proliferation of cells. On the next day AFF4 was diluted to a concentration of 4 μg/mL in NK92 medium supplemented with 3 ng/mL IL-2. In parallel, NK cells were harvested, counted, and resuspended in the same medium. E-plates were removed from the RTCA Analyzer, medium was aspirated (50 μL/well), and the prediluted AFF4 (50 μL/well) and NK cells (50 L/well) were added. NK cells were added at densities corresponding to final effector to target (E:T) cell ratios of either 5:1 or 10:1. The final concentration of AFF4 in the E-plates was 1 μg/mL. Plates were transferred into the RTCA Analyzer and impedance measurements were restarted to monitor effector cell mediated killing of target cells. Data were acquired for 72 hours and then analysed with the xCelligence Immunotherapy Software 1.0. The cell index data recorded by the RTCA Analyzer were normalized to the last time point prior to the addition of effector cells. The normalized cell index was converted to % specific cytolysis, by comparing the normalized cell index obtained from wells containing target cells, effector cells and treatment, against wells containing target and effector cells only. The following formula was used by the xCELLigence RTCA software to calculate % specific cytolysis:

$$\% Specific {Cytolysis}_{st} = ( \frac{1 - {NCI}_{st}}{{NCI}_{Rt}} ) \times 100$$

where NCIₛₜ is the normalized cell index for the sample, and NCI_{Rt} is the average of the normalized cell index for matching reference wells (wells containing target and effector cells only).

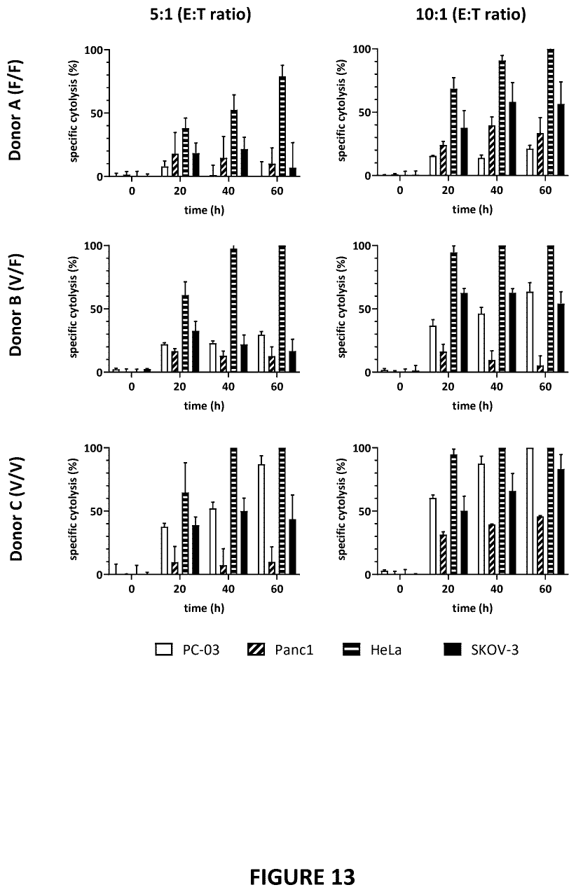

FIG. 13 shows that AFF4 activated effector cells from all three donors and induced specific cytolysis on all tested cell lines. The highest degree of cytotoxicity was observed on the L1CAM-high expressing HeLa cell line, reaching 100% in most of the cases. The degree of specific cytolysis was overall higher at the higher E:T ratio of 10:1 and correlated with the respective CD16 (FcγRIIIA) affinities of effector cells present in the donor cell preparations. Highest activities were observed in the FcγRIIIA V158 homozygous (V/V) donor, intermediate activities in the FcγRIIIA V/F158 heterozygous (V/F) donor, and lowest activities in the FcγRIIIA F158 homozygous (F/F) donor. In the latter donor the best correlation between the cell-surface expression of L1CAM of the different cell lines and the degree of specific cytolysis was observed, with highest levels observed on HeLa and SKOV3 cells, intermediate levels on Panc-1 cells and lowest levels on PC-03 cells.

### Example 7

### Production and Biophysical Characterization of AFF4-WT Antibody Drug Conjugates

For the conjugation to cytotoxic payloads, a version of AFF4 was used, which contains wild type human IgG1 constant domains and thus does not harbour the four mutations (G236A/S239D/A330L/I332E) that are present in the CH2 domain of AFF4. This human IgG1 wild type version of AFF4 is named AFF4-WT.

### Conjugation of AFF4-WT to VA-SG3199 (MP-PEG₈-VA-PABC-SG3199, Tesirine)

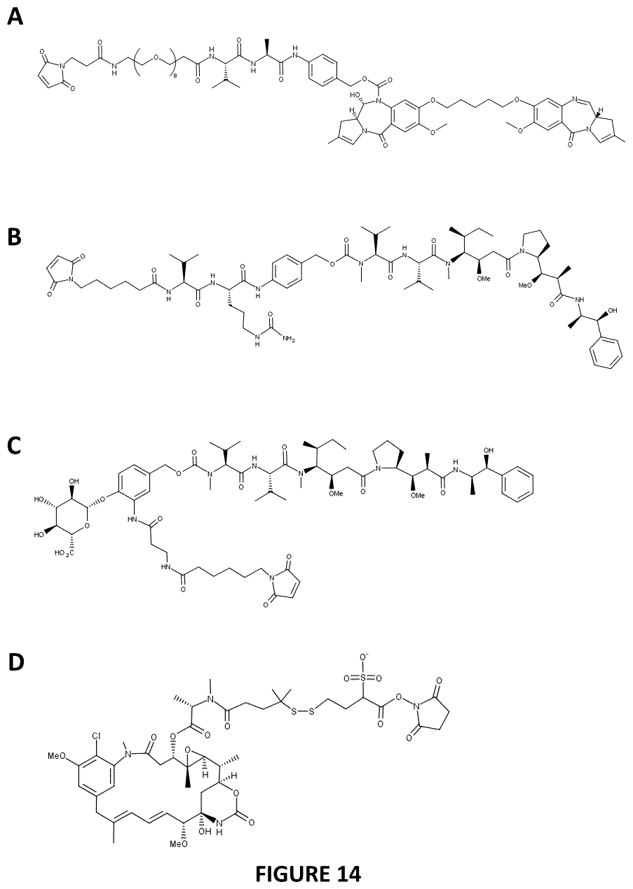

A solution containing AFF4-WT in PBS/100 mM arginine was prepared for reduction by the addition of 5% v/v of 0.5 M Tris-HCl/25 mM EDTA pH 8.5 and then incubated for 2 hours at 30° C. with 1.25 molar equivalents of TCEP (Tris(2-carboxyethyl) phosphine, added from a 1 mM stock in water) to achieve an average of approximately 2 free thiols. The partially reduced AFF4-WT antibody was then conjugated by the addition of 5 molar equivalents of MP-PEG₈-VA-PABC-SG3199 (FIG. 14A). MP-PEG₈-VA-PABC-SG3199 was added from a 10 mM stock in DMA (N,N-dimethylacetamide), with additional DMA added to achieve 5% v/v during the conjugation reaction. After an incubation of 2 hours at room temperature, the reaction was quenched for 30 minutes at room temperature with 5 molar equivalents of N-acetyl-cysteine (added from a 10 mM stock in water), and then desalted and buffer-exchanged to 25 mM histidine/0.2 M sucrose pH 6.0 by passage over a Sephadex G-25 column. For complete removal of free toxin and other low molecular weight additives the AFF4-WT-VA-SG3199 conjugate underwent 8 buffer exchanges of 25 mM histidine/0.2 M sucrose pH 6.0 by discontinuous diafiltration using a PES (polyethersulfone) centrifugal concentrator (Vivaspin) with a 30 kDa molecular weight cut-off. Polysorbate 20 was added from a 1% w/v stock to a final concentration of 0.02% w/v, and the conjugate was sterile filtered using 0.22 μm PVDF (polyvinylidene fluoride) membranes (Millipore Durapore). Aliquots of AFF4-WT-VA-SG3199 were frozen at −80° C.

### Conjugation of AFF4-WT to VC-MMAE (MC-VC-PABC-MMAE, Vedotin), and Gluc-MMAE (MC-Betaglucuronide-MMAE)

A solution containing AFF4-WT in PBS/100 mM arginine was prepared for reduction by the addition of 5% v/v of 0.5 M Tris-HCl/25 mM EDTA PH 8.5 and then incubated for 2 hours at 30° C. with 2.5 molar equivalents of TCEP (added from a 1 mM stock in water) to achieve an average of approximately 4 free thiols. The partially reduced AFF4-WT antibody was then conjugated by the addition of 10 molar equivalents of MC-VC-PABC-MMAE (FIG. 14B) or MC-betaglucuronide-MMAE (FIG. 14C), respectively. MC-VC-PABC-MMAE and MC-betaglucuronide-MMAE were added from 10 mM stocks in DMA, with additional DMA added to achieve 5% v/V during the conjugation reaction. After an incubation of 1 hour at room temperature, the reactions were quenched for 30 minutes at room temperature with 5 molar equivalents of N-acetyl-cysteine (added from a 10 mM stock in water), and then desalted and buffer-exchanged to 25 mM histidine/0.2 M sucrose pH 6.0 by passage over a Sephadex G-25 column. For complete removal of free toxin and other low molecular weight additives, the AFF4-WT-VC-MMAE and AFF4-WT-Gluc-MMAE conjugates underwent 8 buffer exchanges of 25 mM histidine/0.2 M sucrose pH 6.0 by discontinuous diafiltration using a PES centrifugal concentrator (Vivaspin) with a 30 kDa molecular weight cut-off. Polysorbate 20 was added from a 1% w/v stock to a final concentration of 0.02% w/v, and the conjugates were sterile filtered using 0.22 μm PVDF membranes (Millipore Durapore). Aliquots of AFF4-WT-VC-MMAE and AFF4-WT-Gluc-MMAE were frozen at −80° C.

### Conjugation of AFF4-WT to Sulfo-SPDB-DM4

AFF4-WT (in PBS/100 mM arginine) was applied to a protein A column, which had been sanitized with 0.1 M NaOH and equilibrated with PBS pH 7.4. After an extensive washing step (to remove the primary amine arginine) with 20 column volumes of PBS pH 7.4, bound AFF4-WT was eluted by the addition of 0.1 M sodium citrate pH 3.0. The eluted antibody was immediately desalted and buffer exchanged into 50 mM sodium phosphate/150 mM NaCl/2 mM EDTA pH 8.0 by passage over a Sephadex G25 column. AFF4-WT was then concentrated to 5 mg/ml using a PES centrifugal concentrator (Vivaspin) with a 30 kDa molecular weight cut-off. The prepared antibody was then conjugated by the addition of 7 molar equivalents of sulfo-SPDB-DM4 (FIG. 14D). Sulfo-SPDB-DM4 was added from a 10 mM stock in DMA, with additional DMA added to achieve 5% v/v during the conjugation reaction. After 4 hours incubation at room temperature, an additional 1 molar equivalent of sulfo-SPDB-DM4 was added to the reaction and incubation was continued overnight. The conjugation reaction was then quenched by the addition of 1% v/v 0.5 M Tris pH 8.5, and desalted and buffer exchanged into 25 mM histidine/0.2 M sucrose pH 6.0 by passage over a Sephadex G25 column. For complete removal of free toxin and other low molecular weight additives, AFF4-WT-sulfo-SPDB-DM4 underwent 10 buffer exchanges of 25 mM histidine/0.2 M sucrose pH 6.0 by discontinuous diafiltration using a PES centrifugal concentrator (Vivaspin) with a 30 kDa molecular weight cut-off. Polysorbate 20 was added from a 1% w/v stock to a final concentration of 0.02% w/v, and the conjugate was sterile filtered using 0.22 μm PES membranes (Millipore Express). Aliquots of AFF4-WT-sulfo-SPDB-DM4 were frozen and stored at −80° C.

### Biophysical Characterization of AFF4-WT-Drug Conjugates

All AFF4-WT-drug conjugates were analysed for their protein concentration and monomer content (by calculating the area under the curve in size exclusion chromatography at 214 nm), their endotoxin content (by limulus amebocyte lysate assay), and their free drug concentration (by reverse phase chromatography). The drug to antibody ratio of the cysteine conjugates (AFF4-WT-VC-MMAE, AFF4-WT-Gluc-MMAE, AFF4-WT-VA-SG3199) was determined by hydrophobic interaction chromatography, while the drug to antibody ratio of the lysine conjugate (AFF4-WT-sulfo-SPDB-DM4) was determined by size exclusion chromatography through comparison of the areas under the curve obtained at 252 nm (absorbance maximum of DM4) and 280 nm (absorbance maximum of antibody), respectively. Monovalent kinetic binding constants of the different AFF4-WT-drug conjugates towards human L1CAM were determined by surface plasmon resonance essentially as described in Example 4. Results are summarized in Table 14:

TABLE 14
Biophysical characterization of AFF4-WT-drug conjugates

| | | | | AFF4- |
|---|---|---|---|---|
| | AFF4- | AFF4- | AFF4- | WT- |
| | WT- | WT- | WT- | sulfo- |
| | VA- | VC- | Gluc- | SPDB- |
| Conjugate | SG3199 | MMAE | MMAE | DM4 |
| Protein concentration | 3.3 | 3.2 | 3.3 | 3.2 |
| (mg/mL) | | | | |
| % monomer | 98.9 | 99.5 | 99.5 | 99.0 |
| Drug to antibody ratio | 1.8 | 3.8 | 3.9 | 3.7 |
| % free drug | 1 | 0.5 | 0.3 | 0.6 |
| Endotoxin content | 0.6 | 0.28 | 0.32 | 0.16 |
| (EU/mg) | | | | |
| K_{D} (nM) human | 0.14 | 0.14 | 0.14 | 0.16 |
| L1CAM binding | | | | |

### Example 8

### Pharmacological Characterization of AFF4-WT Antibody Drug Conjugates

### Internalization and Lysosomal Localization of AFF4-WT Upon Binding to L1CAM on the Surface of JIMT-1 Breast Cancer, OVCAR-3 Ovarian Cancer and MeWo Melanoma Cells

To exert its cytotoxic effect, an antibody drug conjugate needs to bind to and get efficiently internalized into the lysosomal compartment of cancer cells, where it is processed by specific proteases and its cytotoxic payload is released. To test the internalizing ability of AFF4-WT and track its intracellular localization, Human Fabfluor-pH Red Antibody Labeling Dye was used, an anti-human IgG (Fc specific) Fab fragment, which is conjugated to a pH-sensitive dye that has little fluorescence at neutral pH, but becomes highly fluorogenic at low pH. Upon binding to AFF4-WT, the Fabfluor labeling reagent becomes co-internalized with AFF4-WT and indicates whether the antibody has reached the low pH environment of the lysosomal pathway.

L1CAM expressing JIMT-1 breast cancer, OVCAR-3 ovarian cancer and MeWo melanoma cells were used to determine the lysosomal uptake of AFF4-WT. To this end, cells were seeded at a density of 20′000 cells/well (4×10⁵ cells/ml, 50 L/well) in 96 well cell culture plates. JIMT-1 cells were seeded in DMEM/10% FCS, OVCAR-3 cells in RPMI1640/10% FCS, and MeWo cells in EMEM/10% FCS. After an incubation at 37° C./5% CO₂ of approximately 5 hours, AFF4-WT or a chimeric human IgG1 isotype control antibody, respectively, were mixed with Incucyte® Human Fabfluor-pH Red Antibody Labeling Dye (Sartorius cat no. 4722) and diluted to a concentration of 2 μg/mL each (molar ratio of antibody to labeling dye of 1:3) in RPMI164/10% FCS. After an incubation of 15 min at 37° C., 50 μL/well of the mixture was added to each cell line, yielding at a final in-well concentration of 1 μg/mL of antibody and labelling dye, respectively.

The incubation was continued for 15 min or 20 h, respectively. Thereafter, adherent cells were washed with PBS and then treated for 5 min with Accutase® cell detachment solution (Capricorn), followed by analysis on an image-based cytometer (Nucleocounter NC-3000, ChemoMetec) using an excitation wavelength of 630 nm and an emission filter at 740 nm±60 nm.

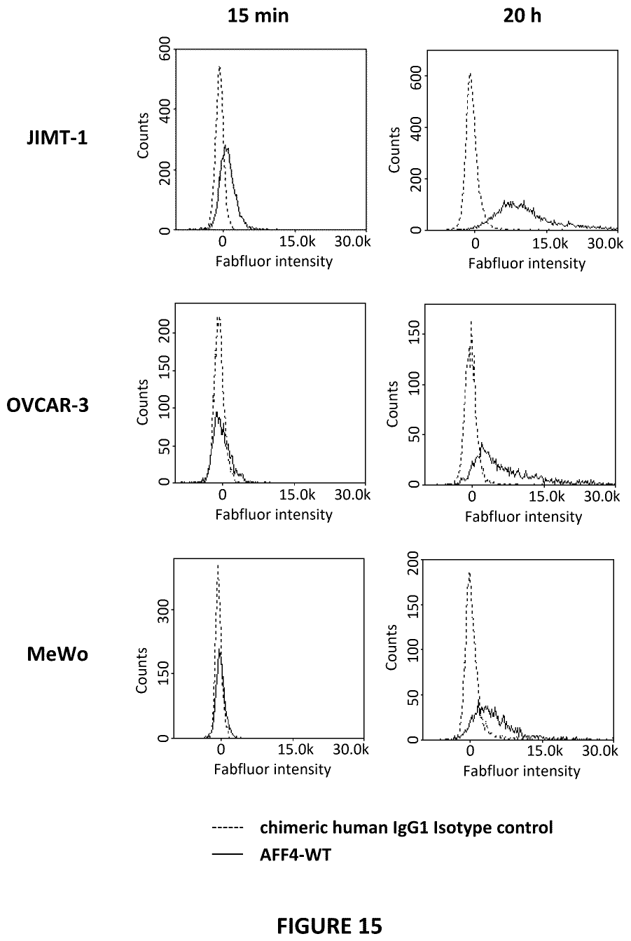

FIG. 15 shows that fluorescence of AFF4-WT treated JIMT-1, OVCAR-3 and MeWo cells was strongly increased after 20 h compared to chimeric human IgG1 isotype control antibody treated cells, indicating that AFF4-WT has been efficiently internalized by all cell lines and has reached the lysosomal compartment.

### Cytotoxicity on L1CAM Expressing Cell Lines In Vitro

The ability of the different AFF4-WT-drug conjugates to induce cytotoxicity was then tested in an in vitro cell-based killing assay. JIMT-1, OVCAR-3, and MeWo cells were seeded at 2′250, 2′100, and 2′000 cells/well, respectively, in a total volume of 90 μL into white 96-well culture plates using the same media as described above for the internalization assay. Cells were allowed to adhere for 6.5 to 7.5 hours, followed by the addition of 10 μL/well of AFF4-WT-drug conjugates that had been serially diluted in RPMI/10% FCS. Final concentrations of conjugates in the assay ranged from 100 nM to 1.3 μM (0.05 μM for AFF4-WT-VA-SG3199). As a positive control for 100% killing, Doxorubicin was added to separate wells at a final concentration of 10 μM. Assay plates were incubated for 6 days at 37° C./5% CO₂ and cell viability was assessed by ATP quantification using CellTiter Glo2.0 (Promega) and luminescence measurement according to the manufacturer's instructions. Luminescence readings were converted to % viability values, using the wells with cells only, and with cells incubated in the presence of 10 μM Doxorubicin, respectively, as 100% and 0% viability reference values. The % viability values obtained at the different AFF4-WT-drug conjugate concentrations were fitted to 4 parameter logistic curves using GraphPad Prism and used to calculate the respective IC₅₀ concentrations of the different conjugates.

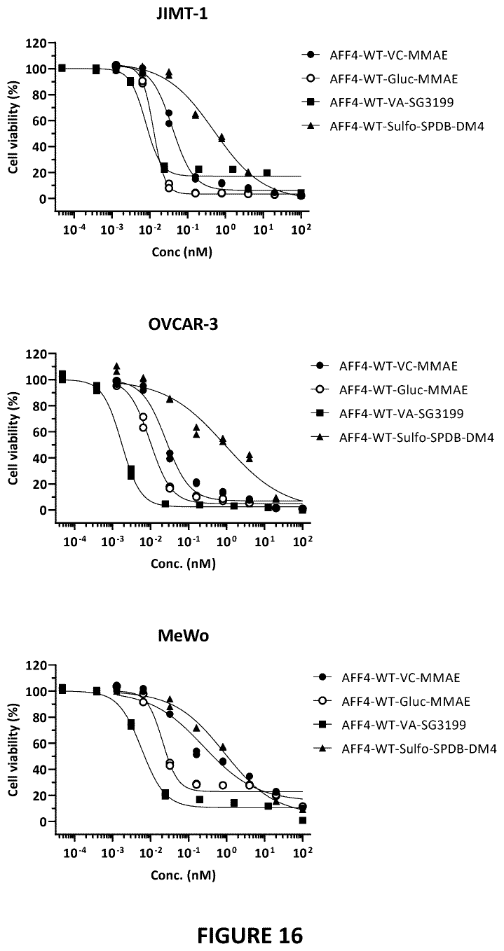

FIG. 16 shows that AFF4-WT-VC-MMAE, AFF4-WT-Gluc-MMAE, AFF4-WT-VA-SG3199, and AFF4-WT-sulfo-SPDB-DM4 all induced dose-dependent killing of JIMT-1, OVCAR-3 and MeWo cells. The respective IC₅₀ values are summarized in Table 15 below.

TABLE 15
Cytotoxicity of AFF4-WT conjugates
on different cancer cell lines¹⁵

| | JIMT-1 | OVCAR-3 | MeWo |
|---|---|---|---|
| AFF4-WT-VC-MMAE | 0.041 | 0.025 | 0.245 |
| AFF4-WT-Gluc-MMAE | 0.013 | 0.010 | 0.021 |
| AFF4-WT-VA-SG3199 | 0.008 | 0.002 | 0.006 |
| AFF4-WT-sulfo-SPDB-DM4 | 0.568 | 0.899 | 0.880 |

¹⁵numbers indicate IC₅₀ values in nM.

### Inhibition of Tumor Growth In Vivo

The in vivo efficacy of the AFF4-WT drug conjugates was determined in mouse xenograft models of the human cancer cell lines JIMT-1, OVCAR-3 and MeWo. JIMT-1 breast cancer cells (5×10⁶ cells in 100 μL PBS) were injected into the left mammary fat pad of female athymic nude (Crl:NU(NCr)-Foxn1^{nu}) mice. Tumors were measured with calipers, and tumor volumes (TV) were calculated using the following formula: TV=(W²×L)/2 (L=length, W=perpendicular length of tumor, L>W).

When tumors had reached an average volume of 100-150 mm³, mice were randomized into 5 different groups. Four groups (n=6 each) received single i.v. injections of either AFF4-WT-VC-MMAE (5 mg/kg), AFF4-WT-Gluc-MMAE (5 mg/kg), AFF4-WT-VA-SG3199 (1 mg/kg), AFF4-WT-sulfo-SPDB-DM4 (5 mg/kg), while the fifth group (n=8) received vehicle as control. Tumor volumes were measured twice weekly following injections.

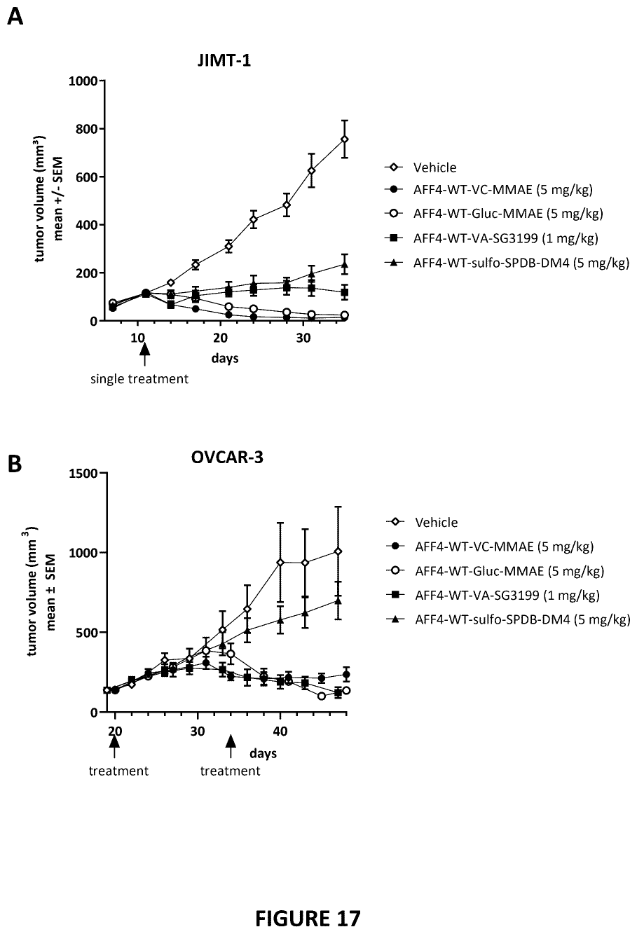

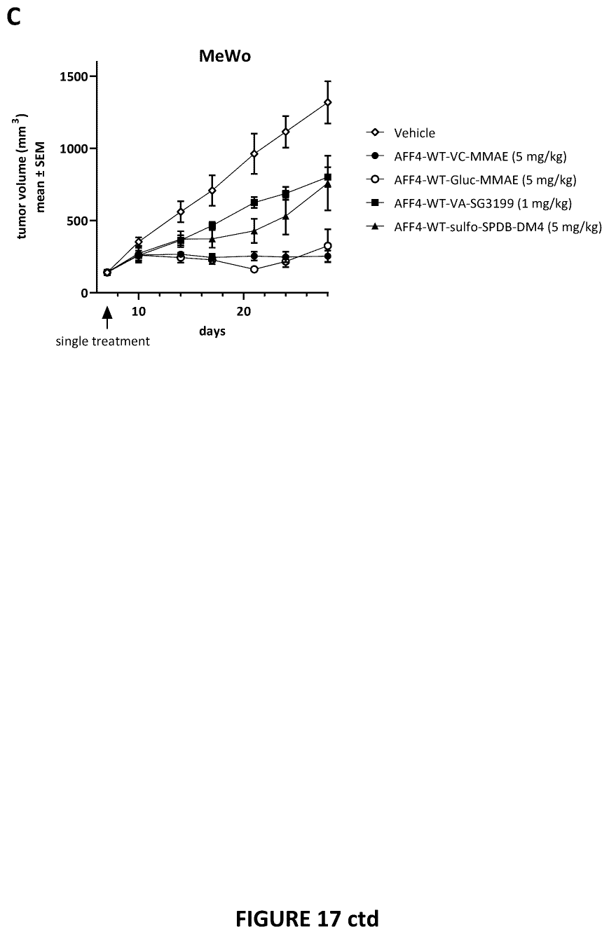

As shown in FIG. 17A, single treatment with either of the AFF4-WT drug conjugates induced strong anti-tumor responses. Tumor regression was observed in mice treated with AFF4-WT-VC-MMAE and AFF4-WT-Gluc-MMAE, respectively, while tumor growth arrest was observed in mice treated with AFF4-WT-VA-SG3199 or AFF4-WT-sulfo-SPDB-DM4.

OVCAR-3 ovarian cancer cells (1×10⁷ cells in Matrigel) were injected subcutaneously into female Jan:NMRI-nu/nu mice. Tumors were measured with calipers and tumor volumes were calculated as described above. Once tumors had reached an average volume of approximately 0.14 cm³, mice were randomized into 5 groups (n=5) and treatment was initiated. Each group received two i.v. injections, two weeks apart, of either AFF4-WT-VC-MMAE (5 mg/kg), AFF4-WT-Gluc-MMAE (5 mg/kg), AFF4-WT-VA-SG3199 (1 mg/kg), AFF4-WT-sulfo-SPDB-DM4 (5 mg/kg), or vehicle as control. Tumor volumes were measured twice weekly after randomization.

FIG. 17B shows that all AFF4-WT-drug conjugates induced anti-tumor responses in the OVCAR-3 xenograft model. Treatment with AFF4-WT-VC-MMAE, AFF4-WT-Gluc-MMAE, or AFF4-WT-VA-SG3199 induced tumor growth arrest, while treatment with AFF4-WT-sulfo-SPDB-DM4 caused a reduction in tumor growth as compared to the vehicle control group.

MeWo melanoma cells (1×10⁷ in Matrigel) were injected subcutaneously into female Jan:NMRI-nu/nu mice. Tumor volumes were determined with calipers as described above. When tumors had reached an average volume of approximately 0.14 cm³, mice were randomized into 5 groups (n=5) and treated with single i.v. injections of either AFF4-WT-VC-MMAE (5 mg/kg), AFF4-WT-Gluc-MMAE (5 mg/kg), AFF4-WT-VA-SG3199 (1 mg/kg), AFF4-WT-sulfo-SPDB-DM4 (5 mg/kg), or vehicle as control. Tumor volumes were measured twice per week after treatment. FIG. 17C shows that anti-tumor responses were observed with all tested AFF4-WT-drug conjugates. Treatment with AFF4-WT-VC-MMAE or AFF4-WT-Gluc-MMAE induced tumor growth arrest, while treatment with AFF4-WT-VA-SG3199 or AFF4-WT-sulfo-SPDB-DM4 caused a decrease in tumor growth.

### Efficacy of Different Doses of AFF4-WT-VC-MMAE in the JIMT-1 Breast Cancer Xenograft Model

JIMT-1 breast cancer cells (5×10⁶ cells in 100 μL PBS) were injected into the left mammary fat pad of female athymic nude (Crl:NU(NCr)-Foxn1^{nu}) mice. When tumors had reached an average volume of approximately 100 mm³, mice were randomized into 5 different groups. Four groups (n=6 each) then received single i.v. injections of AFF4-WT-VC-MMAE at either 0.1, 0.3, 1.0, or 3.0 mg/kg, while the fifth group (n=8) received vehicle as control. Tumor volumes were measured twice weekly following injections.

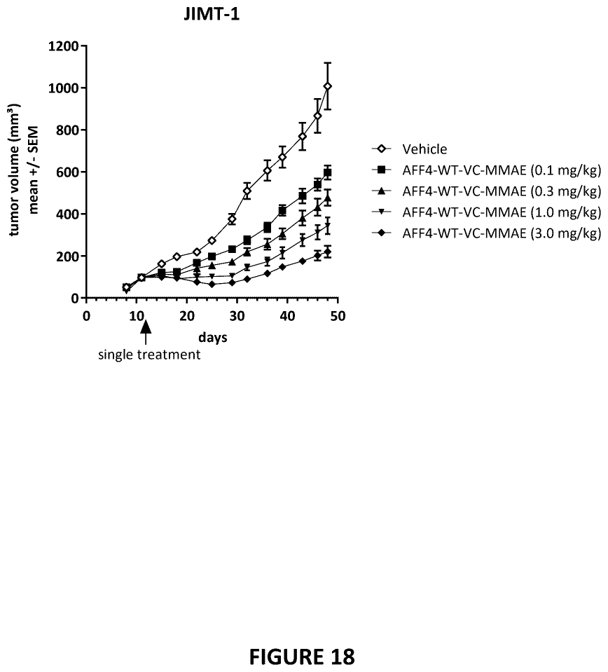

FIG. 18 shows that all doses of AFF4-WT-VC-MMAE inhibited JIMT-1 tumor growth, with a clear dose-response relationship between the tested dose level and the extent of the anti-tumor effect.

### Efficacy of AFF4-WT-VC-MMAE in Patient-Derived Xenograft Models of Ovarian Cancer

The efficacy of AFF4-WT-VC-MMAE was investigated in four patient-derived xenograft (PDX) models of human ovarian cancer with confirmed L1CAM expression. Female athymic Nude-Foxn1nu mice were subcutaneously implanted with fragments of either of the four L1CAM-expressing PDX tumors. Tumor growth was monitored at regular intervals, and when average tumor volumes reached 150-300 mm³, animals were matched by tumor volume into a treatment and a control group (n=5 each). The treatment group then received fortnightly i.v. injections of AFF4-WT-VC-MMAE (3 mg/kg), while the control group received fortnightly injections of vehicle. Tumor volumes were recorded twice weekly, and the degree of tumor growth inhibition (TGI) was calculated for the treatment group (T) versus the control group (C) using initial (i) and final (f) tumor measurements by the formula:

$$\% TGI = ( 1 - \frac{Tf - Ti}{Cf - Ci} ) \times 100$$

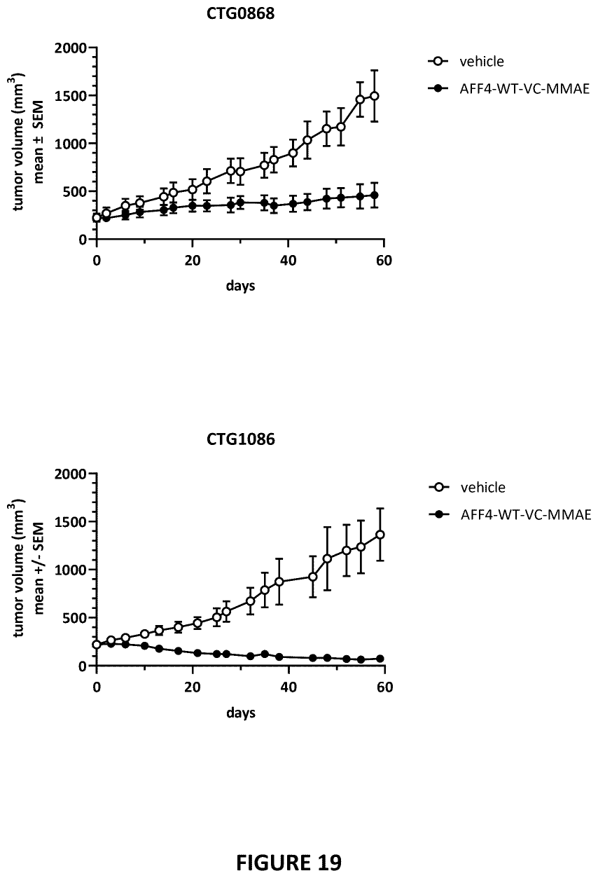

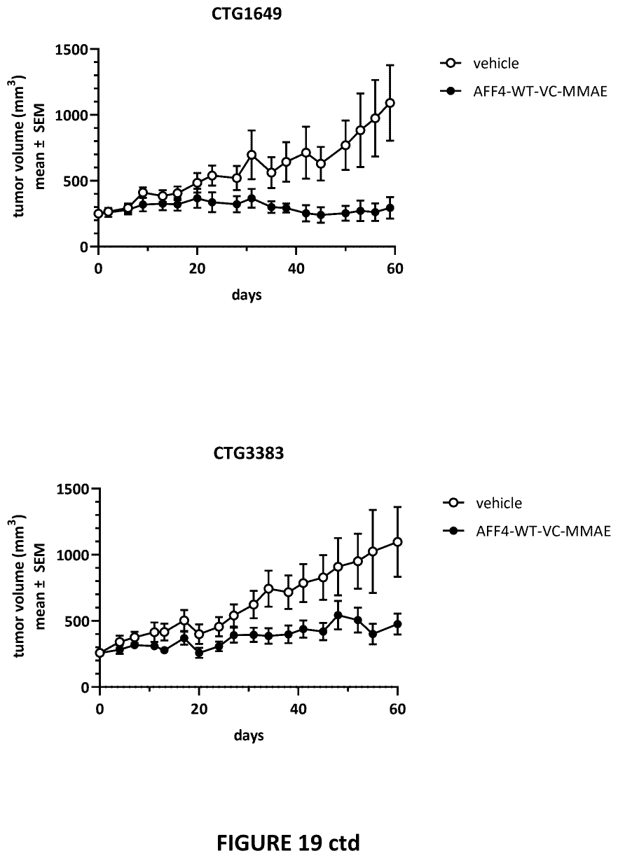

As shown in FIG. 19, AFF4-WT-VC-MMAE inhibited tumor growth in all four L1CAM-expressing ovarian cancer PDX models. The degrees of tumor growth inhibition at the last observation time point were 82% (CTG0868), 113% (CTG1086), 95% (CTG1649), and 74% (CTG3383), respectively.

In Vitro Cytotoxicity of AFF4-WT-VC-MMAE on L1CAM-Expressing Cell Lines and Correlation with L1CAM Expression Levels

A series of 25 ovarian, endometrial, breast cancer, melanoma and neuroblastoma cell lines were seeded at the required densities in a total volume of 90 μL cell culture medium into the wells of white 96 well cell culture plates. Seeding densities and cell culture media were individually determined for each cell line, in order to obtain the optimal assay window. Cells were allowed to adhere for approximately 6 hours, followed by the addition of 10 μL/well of AFF4-WT-VC-MMAE that had been serially diluted in RPMI/10% FCS. Final concentrations of AFF4-WT-VC-MMAE in the assay ranged from 100 nM to 1.3 μM. As a positive control for 100% killing, Doxorubicin was added to separate wells at a final concentration of 10 μM. Assay plates were incubated for 5-12 days at 37° C./5% CO₂ and cell viability was assessed by ATP quantification using CellTiter Glo2.0 (Promega) and luminescence measurement according to the manufacturer's instructions. Luminescence readings obtained at the different AFF4-WT-VC-MMAE concentrations were converted to % viability values as detailed above, and fitted to 4 parameter logistic curves, using GraphPad Prism. IC₅₀ concentrations were derived for each individual cell line.

In parallel, the approximate number of L1CAM molecules expressed on the cell surface of each cancer cell line was determined. To this end, a calibration line was first established using Quantibrite beads labeled with four different densities of Phycoerythrin (PE) molecules (BD Biosciences). Beads were resuspended in 500 μL PBS, and 30 μL of this suspension were loaded on an NC-Slide A2 (ChemoMetec) and analysed using an image-based cytometer (Nucleocounter, ChemoMetec). A green light source (LED530), and an exposure time of 500 ms was used for analysis. After gating the single beads of the four bead populations, mean fluorescence intensities (MFIs) were determined and plotted against the respective numbers of PE molecules per bead. Using GraphPad Prism, a linear regression curve was fitted to the data, using the following equation:

$$y = m \times x + c ,$$

where y is the MFI, and x is the number of PE molecules per bead as provided by the manufacturer. The different cancer cell lines were then grown in their respective cell culture media until they reached a confluence of approximately 80%. Cells were then detached with Accutase, resuspended in PBS, and transferred to two wells of a 96 well polypropylene plate at a density of approximately 3×10⁵ cells/well each. Plates were centrifuged for 5 min at 300 g at 4° C., supernatants were removed, and cell pellets were resuspended in 300 μL PBS/1% FBS, containing 1 μg/mL of either a PE-labeled anti-L1CAM antibody or a PE-labeled isotype control antibody. After an incubation of 1 h at 4° C. in the dark, cells were centrifuged again for 5 min at 300 g and 4° C., and supernatants were removed. Subsequently, cells were washed twice by resuspending in 300 μl PBS, followed by centrifugation (5 min, 300 g, 4° C.) and supernatant removal. After the last centrifugation step, cells were resuspended in 75 μl PBS and 30 μl of the cell suspension were analysed on the Nucleocounter, using the same settings as described above for the determination of the calibration line. Histogram plots of viable cells were generated, and MFI values were inserted as the y values into the linear regression equation established with the PE-labeled beads. The equation was solved for x (=number of PE molecules), and the number of L1CAM molecules per cancer cell was calculated using the following formula:

$$L 1 CAM / cell = \frac{\frac{{MFI}_{L 1 CAM} - c}{m}}{{DAR}_{L 1 CAM}} - \frac{\frac{{MFI}_{isotype} - c}{m}}{{DAR}_{isotype}}$$

where DAR is the dye to antibody ratio of the anti-L1CAM and the isotype control antibody, respectively, as reported by the manufacturer.

Table 16 below shows the numbers of L1CAM molecules on the different cancer cell lines and the corresponding cytotoxic potencies (IC₅₀ values) determined for AFF4-WT-VC-MMAE on the same cell lines:

TABLE 16
L1CAM expression levels and corresponding cytotoxic potencies
of AFF-WT-VC-MMAE on cancer cell lines from different origins¹⁶

| | | | | | Cytotoxic potency of | | |
|---|---|---|---|---|---|---|---|
| | | L1CAM expression | | | AFF4-WT-VC-MMAE | | |
| | | (molecules/cell) | | | (IC₅₀ in pM) | | |
| Cell Line | Cancer type | Mean | SD | n | Mean | SD | n |
| FU-OV-1 | Ovarian | 168′071 | 27702 | 4 | 141 | 44 | 3 |
| JHOS-2 | Ovarian | 117′667 | 8083 | 3 | 175 | 30 | 2 |
| JIMT-1 | Breast | 94′673 | 24969 | 12 | 34 | 16 | 10 |
| OVCAR-3 | Ovarian | 85′571 | 37921 | 4 | 33 | 7 | 3 |
| Caov-3 | Ovarian | 83′653 | 29825 | 6 | 275 | 99 | 3 |
| HEC-251 | Endometrial | 78′216 | 6217 | 3 | 55 | 20 | 3 |
| HEC-108 | Endometrial | 68′342 | 13419 | 3 | 456 | 473 | 2 |
| OVSAHO | Ovarian | 66′750 | 11081 | 4 | 69 | 67 | 7 |
| HEC-1B | Endometrial | 59′450 | 14806 | 6 | 68 | 10 | 3 |
| EFO-27 | Ovarian | 51′850 | 7438 | 4 | 382 | 310 | 5 |
| HHUA | Endometrial | 51′267 | 6045 | 3 | 28 | 19 | 3 |
| MeWo | Melanoma | 38′150 | 1768 | 2 | 962 | 698 | 3 |
| MFE-280 | Endometrial | 26′941 | 24809 | 7 | 38′897 | 11617 | 3 |
| OVKATE | Ovarian | 26′337 | 10539 | 3 | 382 | 11 | 2 |
| SKOV-6 | Ovarian | 22′465 | 10379 | 7 | 15′053 | 11540 | 3 |
| SNGM | Endometrial | 15′179 | 3892 | 3 | 1′905 | 672 | 2 |
| IMR32 | Neuroblastoma | 12′467 | 2335 | 3 | 428 | 160 | 3 |
| SK-BR-3 | Breast | 9′029 | 6550 | 3 | 1′693 | 579 | 3 |
| JHOS-4 | Ovarian | 1′500 | 368 | 2 | 10′100 | 990 | 2 |
| MDA-MB-231 | Breast | 1′305 | — | 1 | 91′630 | — | 1 |
| MDA-MB-468 | Breast | 469 | 821 | 2 | 91′630 | — | 1 |
| JHUEM-1 | Endometrial | 347 | 255 | 2 | 21′550 | 5303 | 2 |
| HEC-1A | Endometrial | 12 | 123 | 2 | 36′885 | 2666 | 2 |
| MDAH-2774 | Ovarian | 0 | 55 | 3 | 32′550 | 2051 | 2 |
| HEC-50B | Endometrial | 0 | 168 | 2 | 27′000 | 424 | 2 |

¹⁶SD: standard deviation, n: number of independent measurements

AFF4-WT-VC-MMAE induced potent cytotoxic effects on a large proportion of the tested cell lines. As shown in table 16, the cytotoxic potency of AFF4-WT-VC-MMAE closely correlated with the number of L1CAM molecules on the cell surface of the respective cell lines. Cell lines with high L1CAM expression were most sensitive to AFF4-WT-VC-MMAE-induced cytotoxic effects (=low IC₅₀), whereas cell lines with lower or absent L1CAM expression were less sensitive (high IC₅₀).

### Bystander Cytotoxicity of AFF4-WT-VC-MMAE

Within human tumors, the expression of L1CAM might occur heterogeneously, with some cancer cells expressing high L1CAM levels and other cells expressing lower levels or no L1CAM at all. As the potency of AFF4-WT-VC-MMAE correlates with the level of L1CAM expression (see Table 16), the question arises whether tumors with heterogeneous L1CAM expression can be efficiently targeted by AFF4-WT-VC-MMAE. It was therefore investigated whether AFF4-WT-VC-MMAE displays a so-called cytotoxic bystander effect. ADCs endowed with such a bystander effect are taken up and processed by antigen-positive cancer cells in a way that releases a form of the cytotoxic payload, which is freely diffusible to neighboring cells, and thus has the ability to kill those cells independently of their antigen expression. Such ADCs are thus well suited for the treatment of tumors with heterogeneous target expression.

To assess bystander activity of AFF4-WT-VC-MMAE, L1CAM high-expressing JIMT-1 breast cancer cells and L1CAM-low expressing MDA-MB-468 best cancer cells, respectively (see Table 16), were seeded each at a density of 5×10⁴ cells/well in a total volume of 400 μL RPMI/10% FCS into 6 wells of a 24-well cell culture plate. Six additional wells were filled with 400 μL RPMI/10% FCS only. After an incubation of 5 hours at 37° C./5% CO₂, AFF4-WT-VC-MMAE was serially diluted in RPMI/10% FCS and 9.8 μL/well of the serial dilutions were added to the seeded wells and to the wells containing cell culture media only. Concentrations of AFF4-WT-VC-MMAE in the assay ranged from 100 nM to 10 μM. One well each of the JIMT-1-seeded wells, the MDA-MB-468-seeded wells, and the wells containing cell culture media only was left untreated as control. The plate was incubated for 4 days at 37° C./5% CO₂.

L1CAM low-expressing MDA-MB468 cells were then seeded at a density of 1500 cells/well in a total volume of 75 μL RPMI/10% FCS into the wells of a white 96 well cell culture plate, followed by an incubation for 4 hours at 37° C./5% CO₂.

The supernatants from all wells of the 24 well plate, that had been incubated for 4 days, were transferred to sterile tubes and centrifuged for 10 min at 2000 rcf. Supernatants were transferred to new sterile tubes, and 25 μL of each supernatant was then added to the seeded MDA-MB-468 cells. As negative control, fresh RPMI/10% FCS medium was added to three wells, and as positive control for maximal growth inhibition, Doxorubicin was added to three wells at a final concentration of 10 μM. After addition of the supernatants, medium and Doxorubicin, the plates were incubated for 6 days at 37° C./5% CO2. Cell viability was then assessed by ATP quantification using CellTiter Glo2.0 (Promega) and luminescence measurement according to the manufacturer's instructions. Luminescence readings were converted to % viability values as detailed above, and fitted to 4 parameter logistic curves, using GraphPad Prism.

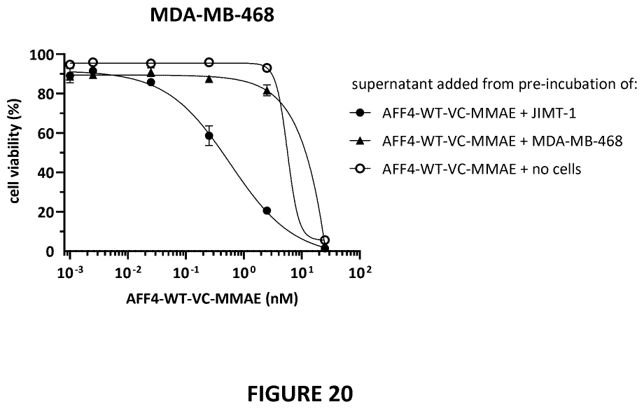

FIG. 20 shows the % viability of MDA-MB-468 cells after incubation with supernatants from JIMT-1 or MDA-MB-468 cells that have been preincubated with serial dilutions of AFF4-WT-VC-MMAE. As control, the % viability obtained by the same serial dilutions of AFF4-WT-VC-MMAE, preincubated in the absence of cells (cell culture medium only), are also shown. Consistent with the virtual insensitivity of MDA-MB-468 to AFF4-WT-VC-MMAE (Table 16), only marginal cytotoxic effects (IC₅₀>5 nM) were observed, when AFF4-WT-VC-MMAE was preincubated on L1CAM low-expressing MDA-MB-468 cells or in the absence of cells. In contrast to that, AFF4-WT-VC-MMAE preincubated on L1CAM high-expressing JIMT-1 cells showed markedly stronger cytotoxic effects on MDA-MB-468 cells, with an IC₅₀ value of approximately 500 μM. This result strongly indicates that uptake and processing of AFF4-WT-VC-MMAE by L1CAM-high expressing JIMT-1 cells produces free MMAE toxin, that is released into the supernatant, where it can exert its cytotoxic action. The data therefore support the notion that AFF4-WT-VC-MMAE displays bystander cytotoxic activity. In the context of a tumor with heterogeneous levels of L1CAM expression, this activity of AFF4-WT-VC-MMAE might ensure that not only high L1CAM expressing tumor cells are killed but also neighboring lower expressing tumor cells are efficiently eliminated, and a good overall anti-tumor response is achieved.

1. An antibody that specifically binds to human L1CAM, comprising:
(a) a heavy chain variable region (VH) complementarity determining region (CDR) 1 comprising the amino acid sequence of GYSITSDYX₁WN (SEQ ID NO: 16), wherein:
X₁ is A or T;
or a sequence containing 1, 2 or 3 amino acid exchanges in the sequence of (SEQ ID NO: 16);
(b) a VH CDR2 comprising the amino acid sequence of YISYSGSX₁SYX₂PSLKS (SEQ ID NO: 17), wherein
X₁ is F or Y, and
X₂ is H or N;
or a sequence containing 1, 2 or 3 amino acid exchanges in the sequence of (SEQ ID NO: 17);
(c) a VH CDR3 comprising the amino acid sequence of SX₁SYX₂YGFAY (SEQ ID NO: 18), wherein:
X₁ is L or F, and
X₂ is G, S or A;
or a sequence containing 1, 2 or 3 amino acid exchanges in the sequence of (SEQ ID NO: 18);
(d) a light chain variable region (VL) CDR1 comprising the amino acid sequence of KASQDVSSAVA (SEQ ID NO: 4);
or a sequence containing 1, 2 or 3 amino acid exchanges in the sequence of (SEQ ID NO: 4);
(e) a VL CDR2 comprising the amino acid sequence of SASYRYX₁ (SEQ ID NO: 19), wherein:
X₁ is T or I;
or a sequence containing 1, 2 or 3 amino acid exchanges in the sequence of (SEQ ID NO: 19); and
(f) a V_{L} CDR3 comprising the amino acid sequence of QQHYSTPWT (SEQ ID NO: 6);
or a sequence containing 1, 2 or 3 amino acid exchanges in the sequence of (SEQ ID NO: 6).

2. An antibody that specifically binds to human L1CAM, comprising:
(a) a heavy chain variable region (VH) complementarity determining region (CDR) 1 comprising the amino acid sequence of GYSITSDYX₁WN (SEQ ID NO: 16), wherein:
X₁ is A or T;
(b) a VH CDR2 comprising the amino acid sequence of YISYSGSX₁SYX₂PSLKS (SEQ ID NO: 17), wherein
X₁ is F or Y, and
X₂ is H or N;
(c) a VH CDR3 comprising the amino acid sequence of SX₁SYX₂YGFAY (SEQ ID NO: 18), wherein:
X₁ is L or F, and
X₂ is G, S or A;
(d) a light chain variable region (VL) CDR1 comprising the amino acid sequence of KASQDVSSAVA (SEQ ID NO: 4);
(e) a VL CDR2 comprising the amino acid sequence of SASYRYX₁ (SEQ ID NO: 19), wherein:
X₁ is T or I; and
(f) a VL CDR3 comprising the amino acid sequence of QQHYSTPWT (SEQ ID NO: 6).

3. The antibody that specifically binds to human L1CAM according to claim 1, wherein:
(a) the VH CDR1 comprises the amino acid sequence of GYSITSDYTWN (SEQ ID NO: 9), or a sequence containing 1, 2 or 3 amino acid exchanges in the sequence of (SEQ ID NO: 9); and/or
(b) the VH CDR2 comprises the amino acid sequence of YISYSGSX₁SYX₂PSLKS (SEQ ID NO: 17), wherein
X₁ is Y, and/or
X₂ is N;
or a sequence containing 1, 2 or 3 amino acid exchanges in the sequence of (SEQ ID NO: 17);
and/or
(c) the VH CDR3 comprises the amino acid sequence of SX₁SYX₂YGFAY (SEQ ID NO: 18), wherein:
X₁ is F, and/or
X₂ is S;
or a sequence containing 1, 2 or 3 amino acid exchanges in the sequence of (SEQ ID NO: 18);
and/or
(d) the VL CDR2 comprises the amino acid sequence of SASYRYT (SEQ ID NO: 5);
or a sequence containing 1, 2 or 3 amino acid exchanges in the sequence of (SEQ ID NO: 5).

4. The antibody that specifically binds to human L1CAM according to claim 1, wherein:
(a) the VH CDR1 comprises the amino acid sequence of GYSITSDYTWN (SEQ ID NO: 9); and/or
(b) the VH CDR2 comprises the amino acid sequence of YISYSGSX₁SYX₂PSLKS (SEQ ID NO: 17), wherein
X₁ is Y, and/or
X₂ is N;
and/or
(c) the VH CDR3 comprises the amino acid sequence of SX₁SYX₂YGFAY (SEQ ID NO: 18), wherein:
X₁ is F, and/or
X₂ is S;
and/or
(d) the VL CDR2 comprises the amino acid sequence of SASYRYT (SEQ ID NO: 5).

5. The antibody that specifically binds to human L1CAM according to claim 1, comprising:
(a) a heavy chain variable region (VH) comprising a VH CDR1 comprising the amino acid sequence of GYSITSDYTWN (SEQ ID NO: 9), or a sequence containing 1, 2 or 3 amino acid exchanges in the sequence of (SEQ ID NO: 9), a VH CDR2 comprising the amino acid sequence of YISYSGSYSYNPSLKS (SEQ ID NO: 11), or a sequence containing 1, 2 or 3 amino acid exchanges in the sequence of (SEQ ID NO: 11), and a VH CDR3 comprising the amino acid sequence of SFSYSYGFAY (SEQ ID NO: 14) or a sequence containing 1, 2 or 3 amino acid exchanges in the sequence of (SEQ ID NO: 14); and
(b) a light chain variable region (VL) comprising a VL CDR1 comprising the amino acid sequence of KASQDVSSAVA (SEQ ID NO: 4), or a sequence containing 1, 2 or 3 amino acid exchanges in the sequence of (SEQ ID NO: 4), a VL CDR2 comprising the amino acid sequence of SASYRYT (SEQ ID NO: 5) or a sequence containing 1, 2 or 3 amino acid exchanges in the sequence of (SEQ ID NO: 5), and a VL CDR3 comprising the amino acid sequence of QQHYSTPWT (SEQ ID NO: 6) or a sequence containing 1, 2 or 3 amino acid exchanges in the sequence of (SEQ ID NO: 6).

6. The antibody that specifically binds to human L1CAM according to claim 1, comprising:
(a) a heavy chain variable region (VH) comprising a VH CDR1 comprising the amino acid sequence of GYSITSDYTWN (SEQ ID NO: 9), a VH CDR2 comprising the amino acid sequence of YISYSGSYSYNPSLKS (SEQ ID NO: 11), and a VH CDR3 comprising the amino acid sequence of SFSYSYGFAY (SEQ ID NO: 14); and
(b) a light chain variable region (VL) comprising a VL CDR1 comprising the amino acid sequence of KASQDVSSAVA (SEQ ID NO: 4), a VL CDR2 comprising the amino acid sequence of SASYRYT (SEQ ID NO: 5), and a VL CDR3 comprising the amino acid sequence of QQHYSTPWT (SEQ ID NO: 6).

7. The antibody according to claim 1, wherein the antibody further comprises a heavy chain variable region sequence comprising the framework regions of the heavy chain variable region sequence of any one of SEQ ID NOs: 23-34, and/or further comprises a light chain variable region sequence comprising the framework regions of the light chain variable region sequence of any one of SEQ ID NOs: 20-22.

8. The antibody of claim 1, wherein the antibody comprises a heavy chain variable region sequence comprising an amino acid sequence selected from the group consisting of SEQ ID NOs: 23-34, and/or comprises a light chain variable region sequence comprising an amino acid sequence selected from the group consisting of SEQ ID Nos: 20-22.

9. The antibody of claim 1, wherein the antibody comprises a heavy chain variable region sequence comprising the amino acid sequence of SEQ ID NO: 30 and/or comprises a light chain variable region sequence comprising the amino acid sequence of SEQ ID NO: 20.

10. The antibody of claim 1, wherein the antibody comprises a heavy chain sequence comprising or consisting of an amino acid sequence of SEQ ID NO: 37 or 84 and/or a light chain sequence comprising or consisting of an amino acid sequence of SEQ ID NO: 38.

11. The antibody of claim 1, comprising:
(a) a heavy chain variable region comprising the amino acid sequence of SEQ ID NO: 30; and
(b) a light chain variable region comprising the amino acid sequence of SEQ ID NO: 20.

12. The antibody of claim 1, wherein the antibody is selected from monoclonal antibodies, recombinantly produced antibodies, monospecific antibodies, multispecific antibodies, including bispecific antibodies, human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, single domain antibodies, monovalent antibodies, single chain antibodies or single-chain Fvs (scFv), camelid antibodies, affibodies, anticalins, affilins, atrimers, DARPins, FN3 scaffolds, such as adnectins and centyrins, fynomers, Kunitz domains, pronectins and OBodies, Fab fragments, F(ab′)2 fragments, disulfide-linked Fvs (dsFv), anti-idiotypic (anti-Id) antibodies, and antigen-binding fragments of any of the above, and/or wherein the antibody is comprised in a Chimeric Antigen Receptor (CAR).

13. The antibody of claim 1, further comprising heavy and/or light chain constant regions, preferably wherein the heavy chain constant region is selected from the group of human immunoglobulins consisting of IgG1, IgG2, IgG3, IgG4, IgA1, and IgA2, and/or, wherein the light chain constant region is selected from the group of human immunoglobulins consisting of IgGκ and IgGλ.

14. The antibody of claim 1, wherein the heavy chain constant region is a variant of a wild type human IgG heavy chain constant region, preferably wherein the variant human IgG heavy chain constant region binds to one or more of human Fc gamma receptors selected from the group consisting of FcγRI, FcγRIIA, FcγRIIIA with higher affinity than the wild type human IgG heavy chain constant region binds to the human Fc gamma receptors.

15. (canceled)

16. The antibody of claim 1, wherein the antibody
(i) binds to human L1CAM within the fibronectin type III domains 1-3 (FN III 1-3) of L1CAM, and/or
(ii) binds to human L1CAM with an affinity (KD) of 20 nM or less, 10 nM or less, or 1 nM or less, and/or
(iii) binds to cynomolgus monkey L1CAM with an affinity (K_{D}) of 20 nM or less, 10 nM or less, or 1 nM or less, and/or
(iv) inhibits the migration of tumor cells on a fibronectin-coated surface in vitro, and/or
(v) inhibits proliferation of SKOV-3, Panc-1, and/or HCT-116 tumor cells in vitro; and/or
(vi) inhibits primary tumor growth in a SKOV-3ip xenograft model, and/or
(vii) reduces metastasis formation in mouse xenograft model MDA-MB231, and/or
(viii) exhibits ADCC activity in vitro and/or binds to FcγRIIIa receptors in vitro; and/or
(ix) exhibits binding to FcRn in vitro, and/or
(x) does not cross-react with human CHL1, human NrCAM, and/or human neurofascin in vitro.

17. The antibody of claim 1, wherein the antibody is a multispecific or bispecific antibody, and/or is a humanized antibody.

18-19. (canceled)

20. The antibody of claim 1, wherein the antibody is linked to at least one therapeutically active substance via a linker.

21. The antibody of claim 20, wherein the therapeutically active substance is selected from the group consisting of a DNA damaging agent, an anti-apoptotic agent, a mitotic inhibitor, an anti-tumor antibiotic, an immunomodulating agent, a nucleic acid for gene therapy, an anti-angiogenic agent, an anti-metabolite, a boron-containing agent, a chemoprotective agent, a hormone agent, an anti-hormone agent, a corticosteroid, a photoactive therapeutic agent, an oligonucleotide, a radioisotope, a radiosensitizer, a topoisomerase inhibitor, and a tyrosine kinase inhibitor.

22. The antibody of claim 21, wherein the mitotic inhibitor is selected from a maytansinoid and an auristatin.

23. The antibody of claim 21, wherein the DNA damaging agent is selected from a pyrrolobenzodiazepine (PBD) and a pyridinobenzodiazepine (PDD).

24. The antibody of claim 20, wherein the linker is a non-cleavable linker or a cleavable linker.

25. (canceled)

26. The antibody of claim 20, wherein the therapeutically active substance is selected from monomethyl auristatin E (MMAE), 4-methyl-4-mercapto-1-oxopentyl)-maytansine (DM4), and VA-SG3199 (tesirine).

27. The antibody of claim 20, wherein
(a) the antibody comprises a heavy chain variable region sequence comprising the amino acid sequence of SEQ ID NO: 30 and/or comprises a light chain variable region sequence comprising the amino acid sequence of SEQ ID NO: 20; or
(b) the antibody comprises a heavy chain variable region sequence comprising the amino acid sequence of SEQ ID NO: 30 and comprises a light chain variable region sequence comprising the amino acid sequence of SEQ ID NO: 20; or
(c) the antibody comprises a heavy chain sequence comprising or consisting of an amino acid sequence of SEQ ID NO: 37 and/or a light chain sequence comprising or consisting of an amino acid sequence of SEQ ID NO: 38; or
(d) the antibody comprises a heavy chain sequence comprising or consisting of an amino acid sequence of SEQ ID NO: 84 and/or a light chain sequence comprising or consisting of an amino acid sequence of SEQ ID NO: 38.

28. A nucleic acid:
(i) coding for an antibody according to claim 1, and/or
(ii) encoding at least one VH or HC and/or VL or LC of an antibody according to claim 1, and/or
(iii) encoding the sequence according to SEQ ID NO: 30 and/or according to SEQ ID NO: 20, and/or
(iv) comprising sequences encoding the complementarity determining region sequences of an antibody according to claim 1,
preferably wherein the nucleic acid is part of a vector.

29. A host cell comprising a nucleic acid according to claim 28.

30. A pharmaceutical composition, comprising an antibody of claim 1, and optionally one or more pharmaceutically acceptable carriers.

31. (canceled)

32. A method of treating or preventing a hyperproliferative disorder, a tumor disease, a disorder associated with neoangiogenesis and/or a disorder associated with aberrant neurogenesis in a patient in need thereof, the method comprising administering to said patient a pharmaceutically effective amount of an antibody of claim 1.

33. A pharmaceutical composition comprising the nucleic acid of claim 28, and optionally one or more pharmaceutically acceptable carriers.

34. A pharmaceutical composition comprising the host cell of claim 29, and optionally one or more pharmaceutically acceptable carriers.

35. A method of treating or preventing a hyperproliferative disorder, a tumor disease, a disorder associated with neoangiogenesis and/or a disorder associated with aberrant neurogenesis in a patient in need thereof, the method comprising administering to said patient a pharmaceutically effective amount of the nucleic acid of claim 28.

36. A method of treating or preventing a hyperproliferative disorder, a tumor disease, a disorder associated with neoangiogenesis and/or a disorder associated with aberrant neurogenesis in a patient in need thereof, the method comprising administering to said patient a pharmaceutically effective amount of the host cell of claim 29.

37. A method of treating or preventing a hyperproliferative disorder, a tumor disease, a disorder associated with neoangiogenesis and/or a disorder associated with aberrant neurogenesis in a patient in need thereof, the method comprising administering to said patient a pharmaceutically effective amount of the pharmaceutical composition of claim 30.
